SEQUENCE LISTING (14 sequences)

SEQ ID NO: 1 - protein, 5 aa, Artificial Sequence
  CDRH1
SYDMS

SEQ ID NO: 2 - protein, 17 aa, Artificial Sequence
  CDRH2
TISGGGSYTYYQDSVKG

SEQ ID NO: 3 - protein, 7 aa, Artificial Sequence
  CDRH3
PYYAMDY

SEQ ID NO: 4 - protein, 11 aa, Artificial Sequence
  CDRL1
KASQDVGTAVA

SEQ ID NO: 5 - protein, 7 aa, Artificial Sequence
  CDRL2
WASTLHT

SEQ ID NO: 6 - protein, 9 aa, Artificial Sequence
  CDRL3
QHYSSYPWT

SEQ ID NO: 7 - protein, 116 aa, Artificial Sequence
  Heavy chain variable region
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSS

SEQ ID NO: 8 - protein, 107 aa, Artificial Sequence
  Light chain variable region
DIQLTQSPSFLSAYVGDRVTITCKASQDVGTAVAWYQQKPGKAPKLLIYWASTLHTGVPSRFSGSGSGTEFTLTISSLQPEDFATYYCQHYSSYPWTFGQGTKLEIK

SEQ ID NO: 9 - protein, 443 aa, Artificial Sequence
  Full heavy chain sequence
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 214 aa, Artificial Sequence
  Full light chain sequence
DIQLTQSPSFLSAYVGDRVTITCKASQDVGTAVAWYQQKPGKAPKLLIYWASTLHTGVPSRFSGSGSGTEFTLTISSLQPEDFATYYCQHYSSYPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 443 aa, Artificial Sequence
  Full heavy chain sequence with N380D modification
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESDGQPENNYKTTPPVLDSDGSFFLYSRLTVDKSRWQEGNVFSCSVMHEALHNHYTQKSLSLSLGK

SEQ ID NO: 12 - protein, 443 aa, Artificial Sequence
  Full heavy chain sequence with N385D modification
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPEDNYKTTPPVLDSDGSFFLYSRLTVDKSRWQEGNVFSCSVMHEALHNHYTQKSLSLSLGK

SEQ ID NO: 13 - protein, 443 aa, Artificial Sequence
  Full heavy chain sequence with N380D and N385D modifications
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESDGQPEDNYKTTPPVLDSDGSFFLYSRLTVDKSRWQEGNVFSCSVMHEALHNHYTQKSLSLSLGK

SEQ ID NO: 14 - protein, 9 aa, Artificial Sequence
  CDRL3
QHYNSYPWT

(19) United States
(12) Patent Application Publication
(10) Pub. No.: US 2023/0059341 A1
(43) Pub. Date: Feb. 23, 2023

(54) BIOPHARMACEUTICAL COMPOSITIONS AND RELATED METHODS

(71) Applicant: Tesaro, Inc., Wilmington, DE (US)

(72) Inventors: Kelly NEELON, Waltham, MA (US); Mingxuan ZHANG, Waltham, MA (US)

(21) Appl. No.: 17/785,506

(22) PCT Filed: Dec. 10, 2020
(86) PCT No.: PCT/US2020/064241

§ 371 Date: Jun. 15, 2022

Related U.S. Application Data
(60) Provisional application No. 62/949,696, filed on Dec. 18, 2019.
(60) Provisional application No. 62/950,595, filed on Dec. 19, 2019.

(51) Int. Cl. C07K 16/28; A61P 35/00

(52) U.S. Cl. CPC C07K 16/2827; A61P 35/00; A61K 2039/505

(57) ABSTRACT

The invention described herein provides compositions comprising anti-PD-1 antibodies and related methods for treating cancer and other disorders responsive to PD-1 antagonism.

### SEQUENCE LISTING

The instant application contains a Sequence Listing which has been submitted electronically in ASCII format and is hereby incorporated by reference in its entirety.

### FIELD OF THE INVENTION

The present disclosure relates to compositions comprising anti-PD-1 antibodies and related methods for treating cancer or infectious conditions and disorders.

### BACKGROUND TO THE INVENTION

Programmed Death 1 (PD-1) (also known as Programmed Cell Death 1) is a type I transmembrane protein of 268 amino acids originally identified by subtractive hybridization of a mouse T cell line undergoing apoptosis. PD-1 is a member of the CD28/CTLA-4 family of T-cell regulators, and is expressed on activated T-cells, B-cells, and myeloid lineage cells.

Two ligands for PD-1 have been identified, PD ligand 1 (PD-L1) and PD ligand 2 (PD-L2), both of which belong to the B7 protein superfamily. PD-L1 is expressed in a variety of cell types, including cells of the lung, heart, thymus, spleen, and kidney. PD-L1 expression is upregulated on macrophages and dendritic cells (DCs) in response to lipopolysaccharide (LPS) and GM-CSF treatment, and on T-cells and B-cells upon signaling via T-cell and B-cell receptors. It is also expressed in a variety of murine tumor cell lines. In contrast, PD-L2 exhibits a more restricted expression pattern and is expressed primarily by antigen presenting cells (e.g. dendritic cells and macrophages), and some tumor cell lines. High PD-L1 expression in tumors, whether on the tumor cell, stroma, or other cells within the tumor microenvironment, correlates with poor clinical prognosis, presumably by inhibiting effector T cells and upregulating regulatory T cells (Treg) in the tumor.

PD-1 negatively regulates T-cell activation, and this inhibitory function is linked to an immunoreceptor tyrosine-based switch motif (ITSM) in the cytoplasmic domain. PD-1 deficiency can also lead to autoimmunity. In humans, a single nucleotide polymorphism in the PD-1 gene is associated with higher incidences of systemic lupus erythematosus, type 1 diabetes, rheumatoid arthritis, and progression of multiple sclerosis. Abnormal PD-1 expression also has been implicated in T-cell dysfunctions in several pathologies, such as tumor immune evasion and chronic viral infections.

Previous studies demonstrate that T-cell suppression induced by PD-1 also plays a role in the suppression of anti-tumor immunity. For example, PD-L1 is expressed on a variety of human and mouse tumors, and binding of PD-1 to PD-L1 on tumors results in T-cell suppression and tumor immune evasion and protection. Expression of PD-L1 by tumor cells has been directly associated with their resistance to lysis by anti-tumor T-cells in vitro. PD-1 knockout mice are resistant to tumor challenge, and T-cells from PD-1 knockout mice are highly effective in tumor rejection when adoptively transferred to tumor-bearing mice. Blocking PD-1 inhibitory signals using a monoclonal antibody can potentiate host anti-tumor immunity in mice, and high levels of PD-L1 expression in tumors are associated with poor prognosis for many human cancer types.

In view of the foregoing, strategies for inhibiting PD-1 activity to treat various types of cancer and for immunopotentiation (e.g. to treat infectious diseases) have been developed. In this respect, monoclonal antibodies targeting PD-1 have been developed for the treatment of cancer. For example, nivolumab (also known as OPDIVO) is a human IgG4 monoclonal antibody directed against PD-1 and marketed to treat melanoma, non-small cell lung cancer or kidney (renal cell) cancer. As another example, pembrolizumab (KEYTRUDA) is a humanized IgG4 monoclonal antibody directed against PD-1 and marketed to treat a range of cancers including non-small cell lung cancer, head and neck cancer, melanoma and Hodgkin lymphoma. In addition, recent evidence suggests that therapies which target PD-1 may enhance immune responses against pathogens, such as HIV. Anti-PD-1 antibodies are described in WO2014/179664, WO2018/085468 and WO2018/129559.

### SUMMARY OF THE INVENTION

According to an aspect of the invention, there is provided a composition comprising an oxidized variant of an anti-PD-1 antibody, wherein the oxidized variant comprises a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises ≤65% of oxidized variant.

According to a further aspect of the invention, there is provided a composition comprising an aggregated variant of an anti-PD-1 antibody, wherein the aggregated variant comprises a heavy chain sequence comprising a CDRH1 of SEQ ID NO: 1, a CDRH2 of SEQ ID NO: 2, and a CDRH3 of SEQ ID NO: 3, and a light chain sequence comprising a CDRL1 of SEQ ID NO: 4, a CDRL2 of SEQ ID NO: 5, and a CDRL3 of SEQ ID NO: 6; wherein the composition comprises ≤36% aggregated variant.

According to a further aspect of the invention, there is provided a composition comprising an antibody having a heavy chain sequence of SEQ ID NO: 9 and a light chain sequence of SEQ ID NO: 10, wherein the composition comprises (i) ≤65% oxidized variant; and/or (ii) ≤36% aggregated variant.

According to a further aspect of the invention, there is provided a composition comprising a charged variant of an anti-PD-1 antibody comprising a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises: ≤100% acidic variant; and/or ≤35% basic variant; and/or ≥1% main isoform.

According to a further aspect of the invention, there is provided a composition comprising a charged variant of an anti-PD-1 antibody comprising a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises: 10-97% acidic variant; and/or 0.1-35% basic variant; and/or 2-80% main isoform.

According to a further aspect of the invention, there is provided a composition comprising a charged variant of an anti-PD-1 antibody comprising a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises: ≤35% acidic variant; and/or ≤5% basic variant; and/or ≥55% main isoform.

According to a further aspect of the invention, there is provided a composition comprising an antibody comprising a heavy chain sequence having one or a combination of sequences selected from SEQ ID NO: 9, SEQ ID NO: 11, SEQ ID NO: 12 and/or SEQ ID NO: 13, and a light chain sequence of SEQ ID NO: 10, wherein the composition comprises ≤64% oxidized variant.

According to a further aspect of the invention, there is provided a composition comprising an antibody comprising a heavy chain sequence having one or a combination of sequences selected from SEQ ID NO: 9, SEQ ID NO: 11, SEQ ID NO: 12 and/or SEQ ID NO: 13, and a light chain sequence of SEQ ID NO: 10, wherein the composition comprises ≤36% aggregated variant.

According to a further aspect of the invention, there is provided a composition comprising a variant of an anti-PD-1 antibody, wherein the variant comprises a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition has at least 60% of the potency of a composition comprising a heavy chain sequence of SEQ ID NO: 9 and a light chain sequence of SEQ ID NO: 10, 10-97% acidic variant, 0.1-35% basic variant, 2-80% main isoform, 4.8% or less light chain W50 oxidized variant, 1% or less heavy chain M34 oxidized variant, 1.2% or less heavy chain M103 oxidized variant, 15.2% or less aggregated variant, 16.7% or less heavy chain M354 oxidized variant, 29.0% or less heavy chain M424 oxidized variant, 47.1% or less heavy chain M248 oxidized variant, 20.8% or less heavy chain D147 isomerized variant, 13.1% or less heavy chain D151 or D167 isomerized variant, 3.1% or less heavy chain D261, D266 or D276 isomerization variant, 4.6% or less fragmented variants, 27.8% or less heavy chain N380 deamidated variant, 27.2% or less heavy chain N385 deamidated variant, about 7.4% or less heavy chain N311 deamidated variant, about 2.0% or less heavy chain N430 deamidated variant, 90% or more heavy chain C-terminal lysine deleted variants (ΔK443), and 1% or less heavy chain N-terminal pyro-glutamate variant.

According to a further aspect of the invention, there is provided a pharmaceutical composition comprising the composition described herein and at least one pharmaceutically acceptable excipient.

According to a further aspect of the invention, there is provided a formulation comprising the pharmaceutical composition described herein comprising the antibody at about 20 mg/mL to about 125 mg/mL and a buffering agent at a pH of about 5.5 to about 6.5.

According to a further aspect of the invention, there is provided a formulation comprising the pharmaceutical composition as described herein, comprising (a) the antibody at about 20 mg/mL to about 125 mg/mL, (b) citrate buffer or histidine buffer at about 10 mM to about 40 mM, (c) arginine at about 80 mM to about 120 mM or trehalose at about 2 to about 10% w/v, (d) sodium chloride at about 20 mM to about 40 mM, and (e) polysorbate 80 at about 0.01% to about 0.1% w/v, at a pH of about 5.5 to about 6.5.

According to a further aspect of the invention, there is provided an injection device comprising the composition as described herein. According to a further aspect of the invention, there is provided an injection device comprising the pharmaceutical composition as described herein. According to a further aspect of the invention, there is provided an injection device comprising the formulation as described herein.

According to a further aspect of the invention, there is provided a cell culture medium comprising the composition described herein.

According to a further aspect of the invention, there is provided an eluate comprising the composition as described herein.

According to a further aspect of the invention, there is provided a method of treating cancer comprising administering to a subject in need thereof a therapeutically effective amount of the composition as described herein. According to a further aspect of the invention, there is provided a method of treating cancer comprising administering to a subject in need thereof a therapeutically effective amount of the pharmaceutical composition as described herein. According to a further aspect of the invention, there is provided a method of treating cancer comprising administering to a subject in need thereof a therapeutically effective amount of the formulation as described herein.

According to a further aspect of the invention, there is provided the composition described herein for use in therapy. According to a further aspect of the invention, there is provided the pharmaceutical composition described herein for use in therapy. According to a further aspect of the invention, there is provided the formulation described herein for use in therapy.

According to a further aspect of the invention, there is provided the composition described herein for use in the treatment of cancer. According to a further aspect of the invention, there is provided the pharmaceutical composition described herein for use in the treatment of cancer. According to a further aspect of the invention, there is provided the formulation described herein for use in the treatment of cancer.

According to a further aspect of the invention, there is provided a use of the composition described herein in the manufacture of a medicament for use in the treatment of cancer. According to a further aspect of the invention, there is provided a use of the pharmaceutical composition described herein in the manufacture of a medicament for use in the treatment of cancer. According to a further aspect of the invention, there is provided a use of the formulation described herein in the manufacture of a medicament for use in the treatment of cancer.

### BRIEF DESCRIPTION OF THE FIGURES

FIG. 1: Representative cIEF Electropherogram for dostarlimab (A: Full Scale, and B: Zoomed Scale)

### DETAILED DESCRIPTION OF THE INVENTION

### General Definitions

The invention described herein provides compositions comprising anti-Programmed Death 1 (PD-1) antibodies and related methods of treatment through inhibition of PD-1 activity. By “programmed death 1 (PD-1) antibody” is meant an antibody that binds specifically to the programmed death 1 protein (PD-1). It will be understood that a composition comprising anti-PD-1 antibodies, as described herein, may also be referred to as a population of anti-PD-1 antibodies as described herein: the phrases being interchangeable. In an embodiment, the PD-1 antibody comprises a heavy chain with the amino acid sequence set forth in SEQ ID NO: 9, and a light chain with the amino acid sequence set forth in SEQ ID NO: 10.

The term “antibody” as used herein in the broadest sense to refer to molecules with an immunoglobulin-like domain (e.g. IgG, IgM, IgA, IgD or IgE) and includes monoclonal, recombinant, polyclonal, chimeric, human, and humanized molecules of this type.

The term, full, whole or intact antibody, used interchangeably herein, refers to a heterotetrameric glycoprotein with an approximate molecular weight of 150,000 daltons. An intact antibody is composed of two identical heavy chains (HCs) and two identical light chains (LCs) linked by covalent disulphide bonds. This H2L2 structure folds to form three functional domains comprising two antigen-binding fragments, known as ‘Fab’ fragments, and a ‘Fc’ crystallisable fragment. The Fab fragment is composed of the variable region at the amino-terminus, variable heavy (VH) or variable light (VL), and the constant region at the carboxyl terminus, CH1 (heavy) and CL (light). The Fc fragment is composed of two domains formed by dimerization of paired CH2 and CH3 regions. The Fc may elicit effector functions by binding to receptors on immune cells or by binding C1q, the first component of the classical complement pathway. The five classes of antibodies IgM, IgA, IgG, IgE and IgD are defined by distinct heavy chain amino acid sequences which are called μ, α, γ, ε and δ respectively, each heavy chain can pair with either a K or λ light chain. The majority of antibodies in the serum belong to the IgG class, there are four isotypes of human IgG, IgG1, IgG2, IgG3 and IgG4, the sequences of which differ mainly in their hinge region.

Fully human antibodies can be obtained using a variety of methods, for example using yeast-based libraries or transgenic animals (e.g. mice) which are capable of producing repertoires of human antibodies. Yeast presenting human antibodies on their surface which bind to an antigen of interest can be selected using FACS (Fluorescence-Activated Cell Sorting) based methods or by capture on beads using labelled antigens. Transgenic animals that have been modified to express human immunoglobulin genes can be immunized with an antigen of interest and antigen-specific human antibodies isolated using B-cell sorting techniques. Human antibodies produced using these techniques can then be characterized for desired properties such as affinity, developability and selectivity.

Monoclonal antibodies may be produced by a eukaryotic cell clone or a prokaryotic cell clone expressing an antibody. Monoclonal antibodies may also be produced by a eukaryotic cell line which can recombinantly express the heavy chain and light chain of the antibody by virtue of having nucleic acid sequences encoding these introduced into the cell. Exemplary methods to produce antibodies from different eukaryotic cell lines such as Chinese Hamster Ovary cells, hybridomas or immortalized antibody cells derived from an animal (e.g. human) are well known to those skilled in the art.

The antibody may be derived, for example, from either rat, mouse, primate (e.g. cynomolgus, Old World monkey or Great Ape), human or other sources such as nucleic acids generated using molecular biology techniques known to those skilled in the art which encode an antibody molecule.

The antibody may comprise a constant region, which may be of any isotype or subclass. The constant region may be of the IgG isotype, for example, IgG1, IgG2, IgG3, IgG4 or variants thereof. In an embodiment, the antibody comprises a constant region derived from IgG4. In an embodiment, a single serine to proline point mutation is present in the hinge region of the IgG4 heavy chain for the purpose of hinge stabilization. This mutation is at the canonical S228 position (Kabat numbering), corresponding to residue 224 in the full-length heavy chain sequence, as sequentially numbered (SEQ ID NO: 9).

The antibody may be either a fully human, a humanized, or a chimeric antibody. In one embodiment, the antibody is a humanized antibody. In one embodiment, the antibody is a monoclonal antibody.

The antibody may comprise one or more modifications including, for example, a mutated constant domain such that the antibody has enhanced effector functions/ADCC and/or complement activation.

The antibody may comprise two immunoglobulin (Ig) heavy chains (“HC”) and two Ig light chains (“LC”). The basic antibody structural unit may comprise, for example, a tetramer of subunits. Each tetramer may include two pairs of polypeptide chains, each pair having one “light” (about 25 kDa) and one “heavy” chain (about 50-70 kDa). The amino-terminal portion of each chain may include a variable region of about 100 to 110 or more amino acids primarily responsible for antigen recognition. This variable region may initially be expressed linked to a cleavable signal peptide. The variable region without the signal peptide may be referred to as a mature variable region. Thus, in one example, a light chain mature variable region may comprise a light chain variable region without the light chain signal peptide. The carboxy-terminal portion of each chain may define a constant region. In one embodiment, the antibody of the compositions described herein is a full-length antibody.

The terms “VH” and “VL” are used herein to refer to the heavy chain variable region and light chain variable region respectively of an antibody.

The mature variable regions of each light/heavy chain pair may form the antibody binding site (also referred to as the antigen binding site). “Antigen binding site” refers to a site on an antibody which is capable of specifically binding to an antigen, this may be a single variable domain, or it may be paired VH/VL domains as can be found on a standard antibody. Thus, an intact antibody may have, for example, two binding sites. Except in bifunctional or bispecific antibodies, the two binding sites can be the same. The chains all may exhibit the same general structure of relatively conserved framework regions (FR) joined by three hypervariable regions, also called complementarity determining regions or “CDRs”. The CDRs from the two chains of each pair may be aligned by the framework regions, enabling binding to a specific epitope. Thus, in one example, from N-terminal to C-terminal, both light and heavy chains comprise the domains FR1, CDR1, FR2, CDR2, FR3, CDR3 and FR4.

Acceptable heavy chain variable region and light chain variable region framework 1, framework 2 and framework 3 regions are readily recognized by those of ordinary skill in the art. Acceptable heavy chain constant regions (including hinge regions) and light chain constant regions are readily recognized by those of ordinary skill in the art as well. Acceptable antibody isotypes are similarly readily recognized by those of ordinary skill in the art.

“CDRs” are defined as the complementarity determining region amino acid sequences of an antibody. These are the hypervariable regions of immunoglobulin heavy and light chains. There are three heavy chain and three light chain CDRs (or CDR regions) in the variable portion of an immunoglobulin. Thus, “CDRs” as used herein refers to all three heavy chain CDRs, all three light chain CDRs, all heavy and light chain CDRs, or at least two CDRs.

Throughout this specification, the terms “CDR”, “CDRL1”, “CDRL2”, “CDRL3”, “CDRH1”, “CDRH2”, “CDRH3” follow the Kabat numbering convention. The amino acid residues in the variable region sequences and full length antibody sequences are numbered sequentially to denote any antibody variant position or post-translational modification variant position, such as an oxidized variant (e.g. W50), a deamidated variant (e.g. N380) or an isomerized variant (e.g. D147).

It will be apparent to those skilled in the art that there are alternative numbering conventions for amino acid residues in variable region sequences and full length antibody sequences. There are also alternative numbering conventions for CDR sequences, for example those set out according to the Chothia et al. (1989) Nature 342: 877-883. The structure and protein folding of the antibody may mean that other residues are considered part of the CDR sequence and would be understood to be so by a skilled person. Other numbering conventions for CDR sequences available to a skilled person include “AbM” (University of Bath) and “contact” (University College London) methods. Table 1 below represents one definition using each numbering convention for each CDR or binding unit. The Kabat numbering scheme is used in Table 1 to number the variable region amino acid sequence. It should be noted that some of the CDR definitions may vary depending on the individual publication used.

TABLE 1

| | Kabat CDR | Chothia CDR | AbM CDR | Contact CDR |
|---|---|---|---|---|
| H1 | 31-35/35A/35B | 26-32/33/34 | 26-35/35A/35B | 30-35/35A/35B |
| H2 | 50-65 | 52-56 | 50-58 | 47-58 |
| H3 | 95-102 | 95-102 | 95-102 | 93-101 |
| L1 | 24-34 | 24-34 | 24-34 | 30-36 |
| L2 | 50-56 | 50-56 | 50-56 | 46-55 |
| L3 | 89-97 | 89-97 | 89-97 | 89-96 |

The terms “variant”, “antibody variant”, “CDR variant” and “post-translational modification variant” refer to a variant antibody sequence wherein at least one amino acid sequence has been changed with respect to the antibody sequence, for example via a post translational modification, a chemical change or a sequence change via at least one deletion, substitution or addition. Some post-translational modifications result in a chemical change which does not change the sequence (e.g. Met and oxidized Met; or Asp and isomarized/iso-Asp; or aggregation) while others result in a sequence change such as the conversion of one amino acid residue into another (e.g. Asn conversion to Asp via deamidation; or lysine deletion). Further post-translational modification variants are described below. A variant antibody sequence which comprises a sequence change may be the result of a designed sequence change or a post-translational modification.

The amino acid replacement or substitution can be conservative, semi-conservative, or non-conservative. Amino acids are broadly grouped as “aromatic” or “aliphatic”. An aromatic amino acid includes an aromatic ring (e.g. histidine, phenylalanine, tyrosine, and tryptophan). Non-aromatic amino acids are broadly grouped as “aliphatic”.

In one embodiment, substitutions are conservative substitutions. It is well recognized in the art that certain amino acid substitutions are regarded as being “conservative”. Amino acids may be further divided into groups based on common side-chain properties and substitutions within groups that maintain all or substantially all of the binding affinity of the antibody are regarded as conservative substitutions. For example, groups of amino acids include: amino acid residues with hydrophobic side chains such as methionine, alanine, valine, leucine and isoleucine; amino acids with neutral, hydrophilic side chains such as cysteine, serine and threonine; amino acids with acidic side chains such as aspartic acid and glutamic acid; amino acids with basic side chains such as asparagine, glutamine, histidine, lysine and arginine; amino acids with residues that influence chain orientation such as glycine and proline; and amino acids with aromatic side chains such as tryptophan, tyrosine and phenylalanine. The antibodies disclosed herein can comprise such “conservative” amino acid substitutions. In an alternative embodiment, an antibody variant comprises at least one substitution whilst retaining the canonical of the antibody.

“Semi-conservative mutations” include amino acid substitutions of amino acids within the broad group (i.e. aromatic or aliphatic), but not within the same side chain sub-group. For example, the substitution of aspartic acid for asparagine, or asparagine for lysine, involves amino acids within the same group (i.e. aliphatic), but different sub-groups. “Non-conservative mutations” involve amino acid substitutions between different groups, for example, lysine for tryptophan, or phenylalanine for serine, etc.

In one embodiment, an antibody variant is an antibody that is at least about 80%, about 85%, about 90%, about 95%, about 96%, about 97%, about 98% or about 99% identical to (i.e. has sequence identity to) the antibody primary sequence. In another embodiment, an antibody variant comprises an antibody comprising a heavy chain amino acid sequence that is at least about 80%, about 85%, about 90%, about 95%, about 96%, about 97%, about 98% or about 99% identical to the amino acid sequence of SEQ ID NO: 9 and/or a light chain amino acid sequence that is at least about 80%, about 85%, about 90%, about 95%, about 96%, about 97%, about 98% or about 99% identical to the amino acid sequence of SEQ ID NO: 10.

“Percent identity” between a query nucleic acid sequence and a subject nucleic acid sequence is the “Identities” value, expressed as a percentage, that is calculated using a suitable algorithm or software, such as BLASTN, FASTA, DNASTAR Lasergene, GeneDoc, Bioedit, EMBOSS needle or EMBOSS infoalign, over the entire length of the query sequence after a pair-wise global sequence alignment has been performed using a suitable algorithm or software, such as BLASTN, FASTA, ClustalW, MUSCLE, MAFFT, EMBOSS Needle, T-Coffee, and DNASTAR Lasergene. Importantly, a query sequence may be described by a nucleic acid sequence identified in one or more claims herein.

“Percent identity” between a query amino acid sequence and a subject amino acid sequence is the “Identities” value, expressed as a percentage, that is calculated using a suitable algorithm or software, such as BLASTP, FASTA, DNASTAR Lasergene, GeneDoc, Bioedit, EMBOSS needle or EMBOSS infoalign, over the entire length of the query sequence after a pair-wise global sequence alignment has been performed using a suitable algorithm/software such as BLASTP, FASTA, ClustalW, MUSCLE, MAFFT, EMBOSS Needle, T-Coffee, and DNASTAR Lasergene. Importantly, a query sequence may be described by an amino acid sequence identified in one or more claims herein.

The query sequence may be 100% identical to the subject sequence, or it may include up to a certain integer number of amino acid or nucleotide alterations as compared to the subject sequence such that the % identity is less than 100%. For example, the query sequence is at least 50, 60, 70, 75, 80, 85, 90, 95, 96, 97, 98, or 99% identical to the subject sequence. Such alterations include at least one amino acid deletion, substitution (including conservative and non-conservative substitution), or insertion, and wherein said alterations may occur at the amino- or carboxy-terminal positions of the query sequence or anywhere between those terminal positions, interspersed either individually among the amino acids or nucleotides in the query sequence or in one or more contiguous groups within the query sequence.

The % identity may be determined across the entire length of the query sequence, including the CDRs. Alternatively, the % identity may exclude one or more or all of the CDRs, for example all of the CDRs are 100% identical to the subject sequence and the % identity variation is in the remaining portion of the query sequence, e.g. the framework sequence, so that the CDR sequences are fixed and intact.

The amino acid sequences which may be useful, and included, in compositions and related methods of the disclosure may have between about 85% to about 100%, about 90% to about 100%, about 95% to about 100%, about 91%, about 92%, about 93%, about 94%, about 95%, about 96%, about 97%, about 98%, about 99% and about 100% identity to the amino acid sequences identified in the disclosure (e.g. to an antibody heavy chain or antibody light chain). In the disclosure, percent identity between the amino acid sequences described may include any discrete subrange of the percent identity ranges recited above (e.g. any range of integer values within a particular range or discrete sub-values within a particular range).

The antibody binds specifically to a target antigen, human PD-1. Exemplary anti-PD-1 antibodies and methods of making the same are disclosed in International Publication No. WO2014/179664 which is incorporated by reference herein in its entirety. Additional exemplary anti-PD-1 antibodies include those described in WO2018/085468 and WO2018/129559, each of which is incorporated by reference herein in its entirety.

The term “specifically binds” or “binds specifically”, as used herein in relation to antibodies means that the antibody binds to a target antigen as well as a discrete domain, or discrete amino acid sequence, within a target antigen with no or insignificant binding to other (for example, unrelated) proteins. This term, however, does not exclude the fact that the antibody may also be cross-reactive with closely related molecules (for example, those with a high degree of sequence identity or from another genera or species). The antibodies described herein may bind to human PD-1 with at least 2, 5, 10, 50, 100, or 1000-fold greater affinity than they bind to closely related molecules.

Affinity, also referred to as “binding affinity”, is the strength of binding at a single interaction site, i.e. of one molecule, e.g. an antibody, to another molecule, e.g. its target antigen, at a single binding site. The binding affinity of an antibody to its target may be determined by equilibrium methods (e.g. enzyme-linked immunoabsorbent assay (ELISA) or radioimmunoassay (RIA)), or kinetics (e.g. surface plasmon resonance analysis using a BIACORE or similar instrument).

The binding affinity (K_{D}) of the antibody-target antigen interaction may be, for example, from about 1 picomolar (pM) to about 100 micromolar (μM) (e.g. from about 1 picomolar (pM) to about 1 nanomolar (nM), from about 1 nM to about 1 micromolar (μM), or from about 1 μM to about 100 μM). In some embodiments, the anti-PD-1 antibody can bind to a PD-1 protein with a K_{D} less than or equal to 1 nanomolar (e.g. 0.9 nM, 0.8 nM, 0.7 nM, 0.6 nM, 0.5 nM, 0.4 nM, 0.3 nM, 0.2 nM, 0.1 nM, 0.05 nM, 0.025 nM, 0.01 nM, 0.001 nM, or a range defined by any two of the foregoing values). In some embodiments, the anti-PD-1 antibody can bind to PD-1 with a K_{D} less than or equal to 200 pM (e.g. 190 pM, 175 pM, 150 pM, 125 pM, 110 pM, 100 pM, 90 pM, 80 pM, 75 pM, 60 pM, 50 pM, 40 pM, 30 pM, 25 pM, 20 pM, 15 pM, 10 pM, 5 pM, 1 pM, or a range defined by any two of the foregoing values).

Alternatively, the K_{D} may be between 1 pM and 1000 pM, such as between 10 pM and 500 pM, for example about 300 pM. The binding affinity of the antibody is determined by the association constant (Kₐ) and the dissociation constant (K_{d}) (K_{D}=K_{d}/Kₐ). The binding affinity may be measured by BIACORE (surface plasmon resonance), for example, by capture of the test antibody onto a protein-A coated sensor surface and flowing target antigen over this surface. Alternatively, the binding affinity can be measured by FORTEBIO, for example, with the test antibody receptor captured onto a protein-A coated needle and flowing target antigen over this surface.

The K_{d} may be 1×10⁻³ Ms⁻¹ or less. The K_{d} may be between 1×10⁻⁵ Ms⁻¹ and 1×10⁻³ Ms⁻¹; or between 1×10⁻⁴ Ms⁻¹ and 1×10⁻³ Ms⁻¹. A slow K_{d} may result in a slow dissociation of the antibody-target antigen complex and improved neutralization of the target antigen.

The term “specific antigen binding activity” as used herein means antigen binding activity as measured by Surface Plasmon Resonance (SPR). PD-1 specific binding activity may be determined by SPR using a BIACORE instrument, for example performed in the binding mode. It is binding activity divided by total protein (e.g. dostarlimab) content in a sample. The term “FcRn binding activity” as used herein means Neonatal Fc (FcRn) Receptor binding activity as measured by Surface Plasmon Resonance (SPR). FcRn binding may be determined using a BIACORE instrument. It is binding activity to the FcRn receptor, divided by the total protein concentration of the sample.

The SPR method for specific antigen binding and FcRn binding uses a reference standard of dostarlimab. The dostarlimab reference standard can be used in assays to obtain system suitability and sample comparability data, to ensure methods are performing appropriately. The reference standard can allow the establishment of a calibration curve and concentrations of the samples are interpolated from the curve.

Potency is herein defined as the inhibitory activity of the anti-PD-1 antibody or the composition as described herein to inhibit ligand (PD-L1) binding to PD-1. This may be measured by specific binding to the antigen PD-1, by a potency assay (e.g. MSD assay), or by a potency bioassay (e.g. a cell-based assay). The potency assay may be a cell-based competitive binding assay, which measures the dose-dependent ability of the antibody or the composition to inhibit PD-L1 ligand binding to PD-1 constitutively expressed by the cells. The potency bioassay may be a cell-based intracellular signalling bioassay, which measures the dose dependent ability of the antibody or the composition to inhibit PD-L1 ligand binding to PD-1 resulting in T-Cell Receptor (TCR) activation and nuclear factor of activated T-cell response element (NFAT-RE) activation. Results can be reported as percent potency relative to reference material (e.g. control sample).

The Meso Scale Discovery (MSD) potency assay can be used which comprises engineered CHO K1 cells constitutively expressing the PD-1 protein. The activity of the antibody or the composition can be assessed using competitive binding, which measures the dose-dependent ability of an antibody to inhibit ligand binding to PD-1 on the CHO K1 cells. The ligand (PD-L1) is used as the ligand in the assay (PD-L1-mFc). The readout can be measured using a specific detection antibody mixture that releases an electrochemiluminescence (ECL) signal that can be quantified. Results can be reported as percent potency relative to reference material (e.g. control sample).

A cell based potency bioassay can be used which was developed using the Promega PD-1/PD-L1 Blockade Bioassay (catalogue #J1250 or J1255). Specifically, PD-1 effector cells (Jurkat T cells (Promega #J1155)) expressing human PD-1 and a luciferase reporter gene driven by a nuclear factor of activated T-cell response element (NFAT-RE) can be co-cultured with PD-L1 artificial antigen presenting cells (aAPC) (CHO-K1 cells (Promega #J1095)) expressing PD-L1. When the two cell types are co-cultured PD-1/PD-L1 interaction inhibits TCR signaling and consequently the NFAT-RE luminescence. Addition of the antibody or the composition releases the inhibitory signal and resulting in TCR activation and NFAT-RE-mediated luminescence. This blocking of the inhibitory signal is dose-dependent, and the resulting luminescence can be quantified using a plate reader. From the signal responses, 4-parameter curves can be generated for both the reference material and antibody/composition samples by plotting relative luciferase unit (RLU) on the y-axis vs. log 2 transformed concentrations on the x axis. The median effective concentration (EC50) values can be interpolated and used to calculate the potency of the antibody/composition sample relative to that of the reference material (e.g. control sample).

The terms “peptide”, “polypeptide”, “protein” and “peptide chain” each refer to a molecule comprising two or more amino acid residues. A peptide may be monomeric or polymeric.

References to “about” as used herein when referring to a measurable value such as an amount, a temporal duration, and the like, is meant to encompass variations of ±20% or ±10%, including ±5%, ±1%, and ±0.1% from the specified value, as such variations are appropriate to perform the disclosed methods.

### Anti-PD-1 Antibodies

The composition may comprise an anti-PD-1 antibody comprising one or more CDRs according to the invention described herein, or one or both of the heavy or light chain variable regions according to the invention described herein, or one or both of the heavy or light chains according to the invention described herein.

In one aspect, the composition comprises an antibody having a heavy chain sequence comprising a CDRH1 comprising the amino acid sequence of SEQ ID NO: 1, a CDRH2 comprising the amino acid sequence of SEQ ID NO: 2, and a CDRH3 comprising the amino acid sequence of SEQ ID NO: 3, and a light chain sequence comprising a CDRL1 comprising the amino acid sequence of SEQ ID NO: 4, a CDRL2 comprising the amino acid sequence of SEQ ID NO: 5, and a CDRL3 comprising the amino acid sequence of SEQ ID NO: 6.

In one embodiment, the anti-PD-1 antibody comprises a heavy chain variable region CDR1 (“CDRH1”) comprising an amino acid sequence with one or two amino acid variation(s) (“CDR variant”) to the amino acid sequence set forth in SEQ ID NO: 1.

In one embodiment, the anti-PD-1 antibody comprises a heavy chain variable region CDR2 (“CDRH2”) comprising an amino acid sequence with five or less, such as four or less, three or less, two or less, or one amino acid variation(s) (“CDR variant”) to the amino acid sequence set forth in SEQ ID NO: 2. In a further embodiment, the CDRH1 comprises an amino acid sequence with one or two amino acid variation(s) to the amino acid sequence set forth in SEQ ID NO: 2.

In one embodiment, the anti-PD-1 antibody comprises a heavy chain variable region CDR3 (“CDRH3”) comprising an amino acid sequence with one or two amino acid variation(s) (“CDR variant”) to the amino acid sequence set forth in SEQ ID NO: 3.

In one embodiment, the anti-PD-1 antibody comprises a light chain variable region CDR1 (“CDRL1”) comprising an amino acid sequence with three or less, such as one or two amino acid variation(s) (“CDR variant”) to the amino acid sequence set forth in SEQ ID NO: 4.

In one embodiment, the anti-PD-1 antibody comprises a light chain variable region CDR2 (“CDRL2”) comprising an amino acid sequence with one or two amino acid variation(s) (“CDR variant”) to the amino acid sequence set forth in SEQ ID NO: 5.

In one embodiment, the anti-PD-1 antibody comprises a light chain variable region CDR3 (“CDRL3”) comprising an amino acid sequence with three or less, such as one or two amino acid variation(s) (“CDR variant”) to the amino acid sequence set forth in SEQ ID NO: 6.

In one embodiment, the anti-PD-1 antibody comprises a CDRH1 comprising an amino acid sequence with up to one amino acid variation to the amino acid sequence set forth in SEQ ID NO: 1; a CDRH2 comprising an amino acid sequence with up to five amino acid variations to the amino acid sequence set forth in SEQ ID NO: 2; a CDRH3 comprising an amino acid sequence with up to one amino acid variation to the amino acid sequence set forth in SEQ ID NO: 3; a CDRL1 comprising an amino acid sequence with up to three amino acid variations to the amino acid sequence set forth in SEQ ID NO: 4; a CDRL2 comprising an amino acid sequence with up to one amino acid variation to the amino acid sequence set forth in SEQ ID NO: 5; and/or a CDRL3 comprising an amino acid sequence with up to three amino acid variations to the amino acid sequence set forth in SEQ ID NO: 6.

In one embodiment, the anti-PD-1 antibody comprises a heavy chain variable region (“VH”) comprising an amino acid sequence with at least about 90%, 91%, 92%, 93%, 94%, 95%, 96%, 97%, 98%, 99% or 100% sequence identity to the amino acid sequence set forth in SEQ ID NO: 7. In one embodiment, the VH comprises an amino acid sequence with at least one amino acid variation to the amino acid sequence set forth in SEQ ID NO: 7, such as between 1 and 5, such as between 1 and 3, in particular up to 2 amino acid variations to the amino acid sequence set forth in SEQ ID NO: 7.

In one embodiment, the anti-PD-1 antibody comprises a light chain variable region (“VL”) comprising an amino acid sequence with at least about 90%, 91%, 92%, 93%, 94%, 95%, 96%, 97%, 98%, 99% or 100% sequence identity to the amino acid sequence set forth in SEQ ID NO: 8. In one embodiment, the VL comprises an amino acid sequence with at least one amino acid variation to the amino acid sequence set forth in SEQ ID NO: 8, such as between 1 and 5, such as between 1 and 3, in particular up to 2 amino acid variations to the amino acid sequence set forth in SEQ ID NO: 8.

In one embodiment, an anti-PD-1 antibody comprises a VH with the amino acid sequence set forth in SEQ ID NO: 7; and a VL with the amino acid sequence set forth in SEQ ID NO: 8.

In one embodiment, the anti-PD-1 antibody comprises a VH comprising an amino acid sequence with at least about 90%, 91%, 92%, 93%, 94%, 95%, 96%, 97%, 98%, 99% or 100% sequence identity to the amino acid sequence set forth in SEQ ID NO: 7; and a VL comprising an amino acid sequence with at least about 90%, 91%, 92%, 93%, 94%, 95%, 96%, 97%, 98%, 99% or 100% sequence identity to the amino acid sequence set forth in SEQ ID NO: 8.

In one embodiment, the anti-PD-1 antibody comprises a heavy chain sequence (“HC”) comprising an amino acid sequence with at least about 90%, 91%, 92%, 93%, 94%, 95%, 96%, 97%, 98%, 99% or 100% sequence identity to the amino acid sequence set forth in SEQ ID NO: 9. In one embodiment, the HC comprises an amino acid sequence with at least one amino acid variation to the amino acid sequence set forth in SEQ ID NO: 9, such as between 1 and 10, such as between 1 and 7, in particular up to 6 amino acid variations to the amino acid sequence set forth in SEQ ID NO: 9. In a further embodiment, the HC comprises one, two, three, four, five, six or seven amino acid variations to the amino acid sequence set forth in SEQ ID NO: 9.

In one embodiment, the anti-PD-1 antibody comprises a light chain region (“LC”) comprising an amino acid sequence with at least about 90%, 91%, 92%, 93%, 94%, 95%, 96%, 97%, 98%, 99% or 100% sequence identity to the amino acid sequence set forth in SEQ ID NO: 10. In one embodiment, the LC comprises an amino acid sequence with at least one amino acid variation to the amino acid sequence set forth in SEQ ID NO: 10, such as between 1 and 10, such as between 1 and 5, in particular up to 3 amino acid variations to the amino acid sequence set forth in SEQ ID NO: 10. In a further embodiment, the LC comprises one, two or three amino acid variations to the amino acid sequence set forth in SEQ ID NO: 10.

In one embodiment, the anti-PD-1 antibody comprises a HC comprising an amino acid sequence with at least about 90%, 91%, 92%, 93%, 94%, 95%, 96%, 97%, 98%, 99% or 100% sequence identity to the amino acid sequence set forth in SEQ ID NO: 9; and a LC comprising an amino acid sequence with at least about 90%, 91%, 92%, 93%, 94%, 95%, 96%, 97%, 98%, 99% or 100% sequence identity to the amino acid sequence set forth in SEQ ID NO: 10. Therefore, the antibody is an antibody with a heavy chain at least about 90% identical to the heavy chain amino acid sequence of SEQ ID NO: 9 and/or with a light chain at least about 90% identical to the light chain amino acid sequence of SEQ ID NO: 10.

In one embodiment, the antibody comprises a heavy chain sequence of SEQ ID NO: 9 and a light chain sequence of SEQ ID NO: 10. In one embodiment, the antibody is dostarlimab comprising a heavy chain sequence of SEQ ID NO: 9 and a light chain sequence of SEQ ID NO: 10.

### Post-Translational Modification Products

The skilled person will appreciate that, upon production of an antibody, post-translational modifications may occur which produces a post-translational modification product. A “post-translational modification variant” of an antibody described herein is an antibody composition wherein all or a portion of the composition comprises a “post-translational modification”. Post-translational modifications are chemical changes to the antibody that may be the result from production of the antibody in a host cell, upstream and/or downstream manufacturing processes, and/or length of storage and storage conditions (e.g. effect of exposure to light, temperature, pH, water, or by reaction with an excipient and/or the immediate container closure system). Therefore, the composition of the invention may be formed from the manufacture or storage of antibodies of the invention. Exemplary post-translational modifications comprise antibody sequence changes (“antibody variant” as described above), cleavage of certain leader sequences, the addition of various sugar moieties in various glycosylation patterns including non-enzymatic glycosylation or glycation; deamidation; oxidation; disulfide bond scrambling and other cysteine variants, such as free sulfhydryls, racemized disulfides, thioethers and trisulfide bonds; isomerization; C-terminal lysine cleavage or clipping; and/or N-terminal glutamine cyclisation.

In one example, a post-translational modification product comprises a “product related impurity” that comprises a chemical change that results in reduced function and/or activity. In another example, a post-translational modification product comprises a “product related substance” that comprises a chemical change that does not result in reduced function and/or activity. Product related impurities for the anti-PD-1 antibodies described herein include oxidized variants and aggregated variants. Product related substances for the anti-PD-1 antibodies described herein include deamidated variants, isomerized variants, C-terminal cleaved variants and N-terminal pyro-glutamate variants.

In one embodiment, the anti-PD-1 antibody is dostarlimab comprising a heavy chain with the amino acid sequence set forth in SEQ ID NO: 9, and a light chain with the amino acid sequence set forth in SEQ ID NO: 10, comprising all functional post-translational modifications thereof.

The percent variant provided herein is expressed as a percentage of the total amount of antibody in the composition (e.g. a “population” of antibodies). For example, 65% or less oxidized variants is in the context of total antibody in the composition being 100%, of which 65% or less is oxidized; it does not include any other non-antibody substances present in the composition which may or may not be oxidized.

Antibody variants are commonly observed when the composition of antibodies is analyzed by charged based-separation techniques such as isoelectric focusing (IEF) gel electrophoresis, capillary isoelectric focusing (cIEF) gel electrophoresis, cation exchange chromatography (CEX) and anion exchange chromatography (AEX).

Post translational modifications can result in an increase or decrease in the net charge of the antibody and cause a decrease or increase in the pI value, thereby leading to acidic variants and basic variants (collectively called “charged variants”) with respect to the main isoform. The “main isoform” is the antibody population that elutes as the major peak on chromatograms. Acidic species are variants with lower apparent pI and basic species are variants with higher apparent pI, when antibodies are analyzed using IEF based methods. When analyzed by chromatography-based methods, acidic species and basic species are defined based on their retention times relative to the main peak. Acidic species are the variants that elute earlier than the main peak from CEX or later then than the main peak from AEX, while basic species are the variants that elute later than the main peak from CEX or earlier than the main peak from AEX. These methods separate the main isoform of the antibody from the acidic isoform (acidic variant) and basic isoform (basic variant).

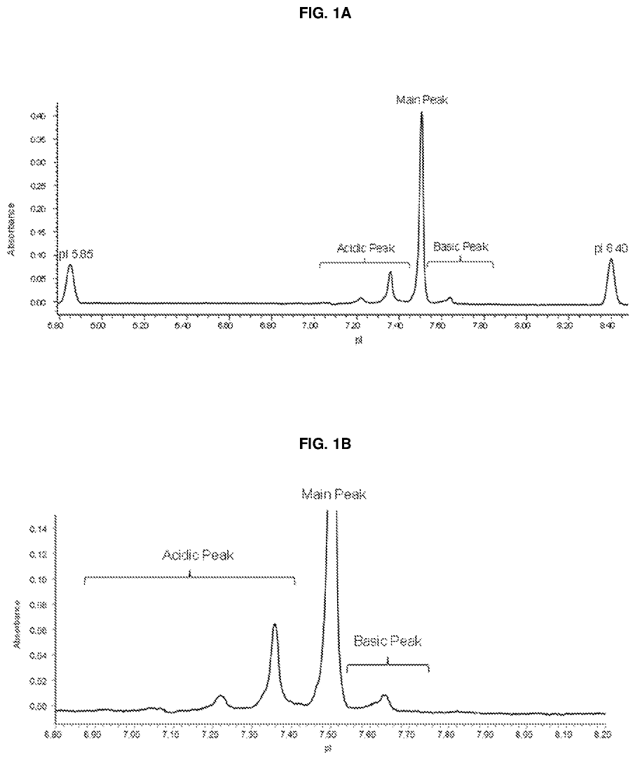

The charged variant can be detected by various methods, such as ion exchange chromatography, for example, WCX-10 HPLC (a weak cation exchange chromatography) or IEF (isoelectric focusing). The percent charged variant can be determined using capillary isoelectric focusing (cIEF). Capillary isoelectric focusing (cIEF) was used to measure the pI of dostarlimab and separate charge variants (see FIG. 1). The method can be used to quantitate the acidic and basic species as a percentage of the total area peak. The terms “species”, “isoform”, “form” and “peak” are used interchangeably to refer to the main isoform and the charged variant (acidic variant and basic variant).

In one aspect, the composition comprises an acidic variant of an anti-PD-1 antibody, wherein the acidic variant comprises a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises ≤100% acidic variant.

In one embodiment, the composition comprises an acidic variant of an anti-PD-1 antibody, wherein the acidic variant comprises a heavy chain variable region at least about 90% identical to the amino acid sequence of SEQ ID NO: 7 and/or a light chain variable region at least about 90% identical to the amino acid sequence of SEQ ID NO: 8; wherein the composition comprises ≤100% acidic variant.

In another embodiment, the composition comprises an acidic variant of an anti-PD-1 antibody, wherein the acidic variant comprises a heavy chain at least about 90% identical to the amino acid sequence of SEQ ID NO: 9 and/or a light chain variable region at least about 90% identical to the amino acid sequence of SEQ ID NO: 10; wherein the composition comprises ≤100% acidic variant. In a yet further embodiment, the composition comprises an acidic variant of an anti-PD-1 antibody, wherein the acidic variant comprises a heavy chain sequence of SEQ ID NO: 9 and a light chain sequence of SEQ ID NO: 10; wherein the composition comprises ≤100% acidic variant. In one embodiment, the composition comprises an acidic variant of dostarlimab, wherein the acidic variant comprises a heavy chain sequence of SEQ ID NO: 9 and a light chain sequence of SEQ ID NO: 10; wherein the composition comprises ≤100% acidic variant.

In one aspect, the composition comprises ≤100% acidic variant. In one embodiment, the composition comprises ≤95%, ≤90%, ≤80%, ≤70%, ≤60%, ≤50%, ≤40%, ≤35%, ≤30% or ≤25% acidic variant. Alternatively, the composition comprises comprises 5-100%, 5-90%, 5-80%, 5-70%, 5-60%, 5-50%, 5-40%, 5-35%, 5-30% or 5-25% acidic variant. Alternatively, the composition comprises 10-100%, 10-97%, 10-90%, 10-80%, 10-70%, 10-60%, 10-50%, 10-40%, 10-35%, 10-30% or 10-25% acidic variant. Alternatively, the composition comprises 20-100%, 20-97%, 20-90%, 20-80%, 20-70%, 20-60%, 20-50%, 20-40%, 20-35%, 20-30% or 20-25% acidic variant. Alternatively, the composition comprises about 60%, about 50%, about 45%, about 40%, about 35%, about 30%, about 25%, about 20% or about 10% acidic variant.

In one aspect, the composition comprises a basic variant of an anti-PD-1 antibody, wherein the basic variant comprises a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises ≤35% basic variant.

In one embodiment, the composition comprises a basic variant of an anti-PD-1 antibody, wherein the basic variant comprises a heavy chain variable region at least about 90% identical to the amino acid sequence of SEQ ID NO: 7 and/or a light chain variable region at least about 90% identical to the amino acid sequence of SEQ ID NO: 8; wherein the composition comprises ≤35% of basic variant. In one embodiment, the composition has at least 60% bioassay potency compared to a reference standard bioassay potency.

In another embodiment, the composition comprises a basic variant of an anti-PD-1 antibody, wherein the basic variant comprises a heavy chain at least about 90% identical to the amino acid sequence of SEQ ID NO: 9 and/or a light chain at least about 90% identical to the amino acid sequence of SEQ ID NO: 10; wherein the composition comprises ≤35% of basic variant. In a yet further embodiment, the composition comprises a basic variant of an anti-PD-1 antibody, wherein the basic variant comprises a heavy chain sequence of SEQ ID NO: 9 and a light chain sequence of SEQ ID NO: 10; wherein the composition comprises ≤35% basic variant. In one embodiment, the composition comprises a basic variant of dostarlimab, wherein the basic variant comprises a heavy chain sequence of SEQ ID NO: 9 and a light chain sequence of SEQ ID NO: 10; wherein the composition comprises ≤35% basic variant.

In one aspect, the composition comprises ≤35% basic variant. In one embodiment, the composition comprises ≤30%, ≤25%, ≤20%, ≤15%, ≤10%, ≤8%, ≤7.5%, ≤7%, ≤6% or ≤5% basic variant. In one embodiment, the composition comprises 0.1-35%, 0.1-30%, 0.1-25%, 0.1-20%, 0.1-15%, 0.1-10%, 0.1-8%, 0.1-7.5%, 0.1-7%, 0.1-6% or 0.1-5% basic variant. In one embodiment, the composition comprises 1-35%, 1-30%, 1-25%, 1-20%, 1-15%, 1-10%, 1-8%, 1-7.5%, 1-7%, 1-6% or 1-5% basic variant. Alternatively, the composition comprises about 35%, about 30%, about 25%, about 20%, about 15%, about 10%, about 7.5% or about 5% basic variant.

In one aspect, the composition comprises a main isoform of an anti-PD-1 antibody, wherein the main isoform comprises a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises ≥1% main isoform.

In one embodiment, the composition comprises a main isoform of an anti-PD-1 antibody, wherein the main isoform comprises a heavy chain variable region at least about 90% identical to the amino acid sequence of SEQ ID NO: 7 and/or a light chain variable region at least about 90% identical to the amino acid sequence of SEQ ID NO: 8; wherein the composition comprises ≥1% main isoform.

In another embodiment, the composition comprises a main isoform of an anti-PD-1 antibody, wherein the main isoform comprises a heavy chain at least about 90% identical to the amino acid sequence of SEQ ID NO: 9 and/or a light chain variable region at least about 90% identical to the amino acid sequence of SEQ ID NO: 10; wherein the composition comprises ≥1% main isoform. In a yet further embodiment, the composition comprises a main isoform of an anti-PD-1 antibody, wherein the main isoform comprises a heavy chain sequence of SEQ ID NO: 9 and a light chain sequence of SEQ ID NO: 10; wherein the composition comprises ≥1% main isoform. In one embodiment, the composition comprises a main isoform of dostarlimab, wherein the main isoform comprises a heavy chain sequence of SEQ ID NO: 9 and a light chain sequence of SEQ ID NO: 10; wherein the composition comprises ≥1% main isoform.

In one aspect, the composition comprises ≥1% main isoform. In one embodiment, the composition comprises ≥2.6%, ≥3%, ≥5%, ≥10%, ≥20%, ≥30%, ≥40%, ≥50%, ≥55%, ≥60%, ≥65%, ≥70%, ≥75%, ≥80% or ≥90% main isoform. In one embodiment, the composition comprises 2-90%, 2-80%, 2-75%, 5-90%, 10-90%, 20-90%, 30-90%, 40-90%, 50-90% or 60-90% main isoform. In one embodiment, the composition comprises 5-80%, 10-80%, 20-80%, 30-80%, 40-80%, 50-80% or 60-80% main isoform. Alternatively, the composition comprises about 80%, about 75%, about 70%, about 65%, about 60%, about 50% or about 55% main isoform.

The percent acidic variant, percent basic variant and percent main isoform can be determined using capillary isoelectric focusing (cIEF). It is understood that these isoform/charged variant embodiments may be combined with any one or a combination of antibody variants described herein.

In one aspect, the composition comprises a charged variant of an anti-PD-1 antibody comprising a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises: ≤100% acidic variant; and/or ≤35% basic variant; and/or 1% main isoform.

In another aspect, the composition comprises a charged variant of an anti-PD-1 antibody comprising a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises: 10-97% acidic variant; and/or 0.1-35% basic variant; and/or 2-80% main isoform.

In another aspect, the composition comprises a charged variant of an anti-PD-1 antibody comprising a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises: ≤35% acidic variant; and/or ≤5% basic variant; and/or ≥55% main isoform.

In another aspect, the composition comprises a charged variant of an anti-PD-1 antibody comprising a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises: 10-30% acidic variant; and/or 0.1-10% basic variant; and/or 60-80% main isoform.

In one aspect, the composition comprises a charged variant of an anti-PD-1 antibody comprising a heavy chain amino acid sequence comprising a VH of SEQ ID NO: 7, and a light chain amino acid sequence comprising a VL of SEQ ID NO: 8; wherein the composition comprises: ≤100% acidic variant; and/or ≤35% basic variant; and/or ≥1% main isoform. In one embodiment, the composition comprises: 10-97% acidic variant; and/or 0.1-35% basic variant; and/or 2-80% main isoform. In an alternative embodiment, the composition comprises: 10-30% acidic variant; and/or 0.1-10% basic variant; and/or 60-80% main isoform. In a further embodiment, the composition comprises: ≤35% acidic variant; and/or 55% basic variant; and/or ≥55% main isoform. In one aspect, the composition comprises a charged variant of an anti-PD-1 antibody comprising a heavy chain amino acid sequence of SEQ ID NO: 9, and a light chain amino acid sequence of SEQ ID NO: 10; wherein the composition comprises: ≤100% acidic variant; and/or ≤35% basic variant; and/or ≥1% main isoform. In one embodiment, the composition comprises: 10-97% acidic variant; and/or 0.1-35% basic variant; and/or 2-80% main isoform. In an alternative embodiment, the composition comprises: 10-30% acidic variant; and/or 0.1-10% basic variant; and/or 60-80% main isoform. In a further embodiment, the composition comprises: ≤35% acidic variant; and/or ≤5% basic variant; and/or ≥55% main isoform. In one embodiment, the composition has at least 60% bioassay potency compared to a reference standard bioassay potency.

Oxidation can occur during production and/or storage (i.e. in the presence of oxidizing conditions) and results in a covalent modification of a protein, induced either directly by reactive oxygen species or indirectly by reaction with secondary by-products of oxidative stress. Oxidation may happen primarily with methionine residues, but may also occur at tryptophan and free cysteine residues. Oxidation can occur in a CDR, in a Fab (non-CDR) region, or in an Fc region.

In one aspect, the composition comprises an antibody comprising an oxidation post-translational modification (“oxidation” or “oxidized”), also referred to herein as an “oxidized variant”. The variant may comprise an oxidized amino acid residue in the heavy chain sequence and/or the light chain sequence, such as a CDR of the heavy chain sequence and/or a CDR of the light chain sequence. The oxidized variant may be present in one or both chains of the heavy chain or light chain.

In one aspect, the composition comprises an oxidized variant of an anti-PD-1 antibody, wherein the oxidized variant comprises a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises ≤65% of oxidized variant.

In one aspect, the composition comprises a population of anti-PD-1 antibodies that includes:

antibodies having a heavy chain amino acid sequence comprising SEQ ID NO: 1 (CDRH1), SEQ ID NO: 2 (CDRH2), and SEQ ID NO: 3 (CDRH3) and a light chain amino acid sequence comprising SEQ ID NO: 4 (CDRL1), SEQ ID NO: 5 (CDRL2) and SEQ ID NO: 6 (CDRL3), and

oxidized variants thereof, wherein ≤65% of the population of antibodies is comprised of the oxidized variants.

In one embodiment, the oxidized variant comprises oxidation at a methionine and/or tryptophan residue in a CDR of the heavy chain sequence and/or a CDR of the light chain sequence. In one embodiment, the oxidized variant comprises oxidation at a methionine and/or tryptophan residue in any one of SEQ ID NOs: 1-6. In a further embodiment, the antibody comprises oxidation at a methionine residue in a CDR of the heavy chain sequence, such as CDRH1 and/or CDRH3. In a further embodiment, the antibody comprises oxidation at a tryptophan residue in a CDR of the light chain sequence, such as CDRL2. In some embodiments, the oxidized variant comprises one or a combination of oxidation at: M34 of CDRH1, M103 of CDRH3 and/or W50 of CDRL2.

It will be understood that reference to a position in the CDR (e.g. M34, M103 or W50) provides the position number in relation to the entire antibody sequence (sequential numbering). Therefore, it will be understood that M34 of CDRH1 refers to the fourth residue of SEQ ID NO: 1, i.e. as underlined: SYDMS (SEQ ID NO: 1). Equally, M103 of CDRH3 refers to the fourth residue of SEQ ID NO: 3, i.e. as underlined: PYYAMDY (SEQ ID NO: 3), and W50 of CDRL2 refers to the first residue of SEQ ID NO: 5, i.e. as underlined: WASTLHT (SEQ ID NO: 5).

In one embodiment, the antibody comprises oxidation at a methionine and/or tryptophan residue in the Fc region of the heavy chain sequence and/or the Fc region of the light chain sequence. In some embodiments, the oxidized variant comprises one or a combination of oxidation at: M248, M354 and/or M424 of the Fc region of the heavy chain sequence.

In one aspect, the composition comprises an antibody that is at least about 90% identical to the heavy chain amino acid sequence of SEQ ID NO: 9 and/or at least about 90% identical to the light chain sequence of SEQ ID NO: 10, and comprises oxidation in the heavy chain sequence, e.g. oxidation at amino acid M34 of CDRH1, M103 of CDRH3, M248 of the Fc region, M354 of the Fc region and/or M424 of the Fc region. In one embodiment, the composition comprises an antibody that is at least about 90% identical to the heavy chain amino acid sequence of SEQ ID NO: 9 and/or at least about 90% identical to the light chain sequence of SEQ ID NO: 10, and comprises oxidation in the light chain sequence, e.g. oxidation at amino acid W50 of CDRL2.

In one embodiment, the antibody comprises a heavy chain variable region at least about 90% identical to the amino acid sequence of SEQ ID NO: 7 and/or a light chain variable region at least about 90% identical to the amino acid sequence of SEQ ID NO: 8. In a further embodiment, the antibody is at least about 90% identical to the heavy chain amino acid sequence of SEQ ID NO: 9 and/or at least about 90% identical to the light chain amino acid sequence of SEQ ID NO: 10. In a yet further embodiment, the antibody comprises a heavy chain sequence of SEQ ID NO: 9 and a light chain sequence of SEQ ID NO: 10.

In one aspect, the composition comprises an anti-PD-1 antibody having a heavy chain sequence comprising a CDRH1 comprising the amino acid sequence of SEQ ID NO: 1, a CDRH2 comprising the amino acid sequence of SEQ ID NO: 2, and a CDRH3 comprising the amino acid sequence of SEQ ID NO: 3, and a light chain sequence comprising a CDRL1 comprising the amino acid sequence of SEQ ID NO: 4, a CDRL2 comprising the amino acid sequence of SEQ ID NO: 5, and a CDRL3 comprising the amino acid sequence of SEQ ID NO: 6; wherein the composition comprises ≤65% oxidized variant.

In one aspect, the composition comprises an anti-PD-1 antibody comprising a heavy chain variable region at least about 90% identical to the amino acid sequence of SEQ ID NO: 7 and/or a light chain variable region at least about 90% identical to the amino acid sequence of SEQ ID NO: 8, wherein the composition comprises ≤65% oxidized variant. In a further aspect, the composition comprises an anti-PD-1 antibody comprising a heavy chain variable region of SEQ ID NO: 7 and/or a light chain variable region of SEQ ID NO: 8, wherein the composition comprises ≤65% oxidized variant.

In one aspect, the composition comprises a population of anti-PD-1 antibodies that includes:

antibodies having a heavy chain variable region as set forth in SEQ ID NO: 7 and a light chain variable region as set forth in SEQ ID NO: 8, and

oxidized variants thereof, wherein ≤65% of the population of antibodies is comprised of the oxidized variants.

In one aspect, the composition comprises an anti-PD-1 antibody comprising a heavy chain sequence at least about 90% identical to the amino acid sequence of SEQ ID NO: 9 and/or a light chain sequence at least about 90% identical to the amino acid sequence of SEQ ID NO: 10, wherein the composition comprises ≤65% oxidized variant.

In one aspect, the composition comprises an anti-PD-1 antibody comprising a heavy chain sequence of SEQ ID NO: 9 and a light chain sequence of SEQ ID NO: 10, wherein the composition comprises ≤65% oxidized variant.

In one embodiment, the composition comprises an oxidized variant of dostarlimab, wherein the oxidized variant comprises a heavy chain sequence of SEQ ID NO: 9 and a light chain sequence of SEQ ID NO: 10; wherein the composition comprises ≤65% oxidized variant.

In one embodiment, the composition comprises an oxidized variant of dostarlimab, wherein the oxidized variant comprises a heavy chain sequence of SEQ ID NO: 9 and a light chain sequence of SEQ ID NO: 10; wherein the composition comprises an amount of oxidized variant in the range of 0.1% to 65%.

In one aspect, the composition comprises (a) an antibody having a heavy chain sequence of SEQ ID NO: 9 and a light chain sequence of SEQ ID NO: 10, and (b) an antibody having a heavy chain sequence at least 90% identical to SEQ ID NO: 9 and a light chain sequence at least 90% identical to SEQ ID NO: 10, wherein the composition comprises ≤65% oxidized variant.

In one aspect, the composition comprises a population of anti-PD-1 antibodies that includes:

antibodies having a heavy chain amino acid sequence as set forth in SEQ ID NO: 9 and a light chain amino acid sequence as set forth in SEQ ID NO: 10, and

oxidized variants thereof, wherein ≤65% of the population of antibodies is comprised of the oxidized variants.

In one aspect, the composition comprises (a) an antibody having a heavy chain sequence of SEQ ID NO: 9 and a light chain sequence of SEQ ID NO: 10, and (b) an antibody having a heavy chain sequence at least 90% identical to SEQ ID NO: 9 and a light chain sequence at least 90% identical to SEQ ID NO: 10; and (c) an oxidized variant of the antibody of (a) and/or (b), wherein the oxidized variant is selected from any one or a combination of ≤34% oxidation at W50 of the light chain, ≤21% oxidation at M34 of the heavy chain, and/or ≤64% oxidation at M103 of the heavy chain.

In one aspect, the composition comprises ≤65% oxidized variant. In one embodiment, the composition comprises ≤65%, ≤60%, ≤50%, ≤40%, ≤30%, ≤20%, ≤15%, ≤10%, ≤5%, ≤4%, or ≤3% oxidized variant. In one embodiment, the composition comprises 0.01-65%, 0.01-60%, 0.01-50%, 0.01-40%, 0.01-30%, 0.01-20%, 0.01-15%, 0.01-10%, 0.01-5%, 0.01-4%, or 0.01-3% oxidized variant. Alternatively, the composition comprises 0.05-65%, 0.05-60%, 0.05-50%, 0.05-40%, 0.05-30%, 0.05-20%, 0.05-15%, 0.05-10%, 0.05-5%, 0.05-4%, or 0.05-3% oxidized variant. Alternatively, the composition comprises 0.5-65%, 0.5-60%, 0.5-50%, 0.5-40%, 0.5-30%, 0.5-20%, 0.5-15%, 0.5-10%, 0.5-5%, 0.5-4%, or 0.5-3% oxidized variant. Alternatively, the composition comprises 1-65%, 1-60%, 1-50%, 1-40%, 1-30%, 1-20%, 1-15%, 1-10%, 1-5%, 1-4%, 1-3%, 2-4%, or 2-3% oxidized variant. Alternatively, the composition comprises about 10%, about 5%, about 4%, about 3%, about 2%, or about 1% oxidized variant. It will be understood that these oxidized variant embodiments may be combined with any one of the antibody variants described herein.

In one embodiment, the composition comprises one or a combination of: ≤21% oxidation at M34 of CDRH1, 564% oxidation at M103 of CDRH3, and/or ≤34% oxidation at W50 of CDRL2. In another embodiment, the composition comprises one or a combination of: ≤16% oxidation at M34 of CDRH1, ≤47% oxidation at M103 of CDRH3, and/or ≤25% oxidation at W50 of CDRL2.

In one aspect, the composition comprises: (a) an antibody having a heavy chain sequence of SEQ ID NO: 9 and a light chain sequence of SEQ ID NO: 10, and (b) an oxidized variant of the antibody selected from any one or a combination of ≤34% oxidation at W50 of the light chain, ≤21% oxidation at M34 of the heavy chain, and/or ≤64% oxidation at M103 of the heavy chain.

In one embodiment, the composition comprises ≤34% oxidation at W50 of the light chain sequence. In one embodiment, the composition comprises ≤34%, ≤30%, ≤25%, ≤20%, ≤15%, ≤10%, ≤7.5%, ≤5%, ≤4%, ≤3%, ≤2%, or ≤1% oxidation at W50 of the light chain sequence. Alternatively, the composition comprises 0-34%, 0-30%, 0-25%, 0-20%, 0-15%, 0-10%, 0-7.5%, 0-5%, 0-4%, 0-3%, 0-2% or 0-1% oxidation at W50 of the light chain sequence. In one embodiment, the composition comprises 0.01-34%, 0.01-30%, 0.01-25%, 0.01-20%, 0.01-15%, 0.01-10%, 0.01-7.5%, 0.01-5%, 0.01-4%, 0.01-3%, 0.01-2%, or 0.01-1% oxidation at W50 of the light chain sequence. In one embodiment, the composition comprises 0.05-34%, 0.05-30%, 0.05-25%, 0.05-20%, 0.05-15%, 0.05-10%, 0.05-7.5%, 0.05-5%, 0.05-4%, 0.05-3%, 0.05-2%, or 0.05-1% oxidation at W50 of the light chain sequence. Alternatively, the composition comprises 0.5-34%, 0.5-30%, 0.5-25%, 0.5-20%, 0.5-15%, 0.5-10%, 0.5-7.5%, 0.5-5%, 0.5-4%, or 0.5-3%, 0.5-2% or 0.5-1% oxidation at W50 of the light chain sequence. Alternatively, the composition comprises 0.1% or more and 34% or less oxidation at W50 of the light chain sequence. Alternatively, the composition comprises about 10%, about 5%, about 4%, about 3%, about 2%, or about 1% oxidation at W50 of the light chain sequence. As shown by the data presented herein, photolysis forced degradation producing up to 4.8% oxidation at W50 gives 90% potency in bioassay (i.e. within assay variability—full function) and 2,2′-Azobis(2-amidinopropane) dihydrochloride (AAPH) forced degradation producing up to 38.9% oxidation at W50 leads to 44% potency (bioassay). Extrapolating from the AAPH forced degradation data for oxidation at LC Trp 50, up to 34% oxidation can result in at least 60% potency, and up to 25% oxidation can result in at least 70% potency. This is calculated using a linear slope for <1% (control), <1% (T0), 38.9% (1 day), 74.3% (3 day) and 86.8% (5 day) AAPH oxidation samples, which have 98%, 102%, 44%, 14% and 5% potency (bioassay), respectively.

In one aspect, the composition comprises an antibody having a heavy chain sequence of SEQ ID NO: 9 and a light chain sequence of SEQ ID NO: 10, wherein the composition comprises ≤34% oxidation at W50 of the light chain sequence. In one embodiment, the composition comprises an oxidized variant of dostarlimab, wherein the oxidized variant comprises a heavy chain sequence of SEQ ID NO: 9 and a light chain sequence of SEQ ID NO: 10; wherein the composition comprises ≤34% oxidation at W50 of the light chain sequence.

In one embodiment, the composition comprises ≤21% oxidation at M34 of the heavy chain sequence. In one embodiment, the composition comprises ≤21%, ≤20%, ≤16%, ≤15%, ≤12.5%, ≤10%, ≤7.5%, ≤5%, ≤4%, ≤3%, ≤2%, or ≤1% oxidation at M34 of the heavy chain sequence. Alternatively, the composition comprises 0-21%, 0-20%, 0-16%, 0-15%, 0-12.5%, 0-10%, 0-7.5%, 0-5%, 0-4%, 0-3%, 0-2% or 0-1% oxidation at M34 of the heavy chain sequence. In one embodiment, the composition comprises 0.01-21%, 0.01-20%, 0.01-16%, 0.01-15%, 0.01-12.5%, 0.01-10%, 0.01-7.5%, 0.01-5%, 0.01-4%, 0.01-3%, 0.01-2%, or 0.01-1% oxidation at M34 of the heavy chain sequence. Alternatively, the composition comprises 0.5-21%, 0.5-20%, 0.5-16%, 0.5-15%, 0.5-12.5%, 0.5-10%, 0.5-7.5%, 0.5-5%, 0.5-4%, 0.5-3%, 0.5-2% or 0.5-1% oxidation at M34 of the heavy chain sequence. Alternatively, the composition comprises 0.1% or more and 21% or less oxidation at M34 of the heavy chain sequence. Alternatively, the composition comprises about 10%, about 5%, about 4%, about 3%, about 2%, or about 1% oxidation at M34 of the heavy chain sequence. As shown by the data presented herein, H₂O₂ forced degradation producing up to 28.8% oxidation at M34 gives 47% potency in bioassay. Extrapolating from the H₂O₂ forced degradation data for oxidation at HC Met 34, up to 21% oxidation can result in at least 60% potency and up to 16% oxidation can result in at least 70% potency. This is calculated using a linear slope for <1% (control), <1% (T0) and 28.8% (H₂O₂ at 2 weeks) H₂O₂ oxidation samples, which have 98%, 94% and 47% potency (bioassay), respectively. In one aspect, the composition comprises an antibody having a heavy chain sequence of SEQ ID NO: 9 and a light chain sequence of SEQ ID NO: 10, wherein the composition comprises ≤21% oxidation at M34 of the heavy chain sequence. In one embodiment, the composition comprises an oxidized variant of dostarlimab, wherein the oxidized variant comprises a heavy chain sequence of SEQ ID NO: 9 and a light chain sequence of SEQ ID NO: 10; wherein the composition comprises ≤21% oxidation at M34 of the heavy chain sequence.

In one embodiment, the composition comprises ≤64% oxidation at M103 of the heavy chain sequence. In one embodiment, the composition comprises ≤64%, ≤60%, ≤50%, ≤47%, ≤40%, ≤30%, ≤20%, ≤15%, ≤10%, ≤5%, ≤2%, or ≤1% oxidation at M103 of the heavy chain sequence. In one embodiment, the composition comprises 0-64%, 0-60%, 0-50%, 0-47%, 0-40%, 0-30%, 0-20%, 0-15%, 0-10%, 0-5%, 0-4%, 0-3%, 0-2%, or 0-1% oxidation at M103 of the heavy chain sequence. In one embodiment, the composition comprises 0.01-64%, 0.01-60%, 0.01-50%, 0.01-47%, 0.01-40%, 0.01-30%, 0.01-20%, 0.01-15%, 0.01-10%, 0.01-5%, 0.01-4%, 0.01-3%, 0.01-2%, or 0.01-1% oxidation at M103 of the heavy chain sequence. Alternatively, the composition comprises 0.5-64%, 0.5-60%, 0.5-50%, 0.5-47%, 0.5-40%, 0.5-30%, 0.5-20%, 0.5-15%, 0.5-10%, 0.5-5%, 0.5-4%, 0.5-3%, 0.5-2% or 0.5-1% oxidation at M103 of the heavy chain sequence. Alternatively, the composition comprises 0.1% or more 64% or less oxidation at M103 of the heavy chain sequence. Alternatively, the composition comprises about 10%, about 5%, about 4%, about 3%, about 2%, or about 1% oxidation at M103 of the heavy chain sequence. As shown by the data presented herein, H₂O₂ forced degradation producing up to 86.1% oxidation at M103 gives 47% potency in bioassay. Extrapolating from the H₂O₂ forced degradation data for oxidation at HC Met 103, up to 64% oxidation can result in at least 60% potency and up to 47% oxidation can result in at least 70% potency. This is calculated using a linear slope for <1% (control), 1.2% (T0) and 86.1% (H₂O₂ at 2 weeks) H₂O₂ oxidation samples, which have 98%, 94% and 47% potency (bioassay), respectively.

In one aspect, the composition comprises an antibody having a heavy chain sequence of SEQ ID NO: 9 and a light chain sequence of SEQ ID NO: 10, wherein the composition comprises ≤64% oxidation at M103 of the heavy chain sequence. In one embodiment, the composition comprises an oxidized variant of dostarlimab, wherein the oxidized variant comprises a heavy chain sequence of SEQ ID NO: 9 and a light chain sequence of SEQ ID NO: 10; wherein the composition comprises ≤64% oxidation at M103 of the heavy chain sequence.

In one embodiment, the composition comprises ≤65% oxidation at M248 of the heavy chain sequence. In one embodiment, the composition comprises ≤65%, ≤60%, ≤50%, ≤45%, ≤40%, ≤35%, ≤30%, ≤20%, ≤15%, ≤10%, ≤5%, ≤4%, or ≤3% oxidation at M248 of the heavy chain sequence. In one embodiment, the composition comprises 0.01-65%, 0.01-60%, 0.01-50%, 0.01-40%, 0.01-30%, 0.01-20%, 0.01-15%, 0.01-10%, 0.01-5%, 0.01-4%, 0.01-3%, 0.01-2% or 0.01-1% oxidation at M248 of the heavy chain sequence. Alternatively, the composition comprises 0.5-65%, 0.5-60%, 0.5-50%, 0.5-40%, 0.5-30%, 0.5-20%, 0.5-15%, 0.5-10%, 0.5-5%, 0.5-4%, or 0.5-3% oxidation at M248 of the heavy chain sequence. Alternatively, the composition comprises 1-65%, 1-60%, 1-50%, 1-40%, 1-30%, 1-20%, 1-15%, 1-10%, 1-5%, 1-4%, 1-3%, 2-4%, or 2-3% oxidation at M248 of the heavy chain sequence. Alternatively, the composition comprises 1% or more and 65% or less oxidation at M248 of the heavy chain sequence. Alternatively, the composition comprises about 10%, about 5%, about 4%, about 3%, about 2%, or about 1% oxidation at M248 of the heavy chain sequence. As shown by the data presented herein, H₂O₂ forced degradation producing up to 47.1% oxidation at M248 gives 94% potency in bioassay (i.e. within assay variability—full function) and photolysis forced degradation producing up to 33.4% oxidation at M248 gives 90% potency in bioassay (i.e. within assay variability—full function). It is therefore expected that oxidation at M248 can go higher than 47.1% without any impact to relative potency or FcRn binding.

In one embodiment, the composition comprises ≤65% oxidation at M354 of the heavy chain sequence. In one embodiment, the composition comprises ≤65%, ≤60%, ≤50%, ≤40%, ≤30%, ≤20%, ≤15%, ≤10%, ≤5%, ≤2%, or ≤1% oxidation at M354 of the heavy chain sequence. In one embodiment, the composition comprises 0.01-65%, 0.01-60%, 0.01-50%, 0.01-40%, 0.01-30%, 0.01-20%, 0.01-15%, 0.01-10%, 0.01-5%, 0.01-4%, 0.01-3%, 0.01-2% or 0.01-1% oxidation at M354 of the heavy chain sequence. Alternatively, the composition comprises 0.5-65%, 0.5-60%, 0.5-50%, 0.5-40%, 0.5-30%, 0.5-20%, 0.5-15%, 0.5-10%, 0.5-5%, 0.5-4%, or 0.5-3% oxidation at M354 of the heavy chain sequence. Alternatively, the composition comprises 0.1% or more and 65% or less oxidation at M354 of the heavy chain sequence. Alternatively, the composition comprises about 10%, about 5%, about 4%, about 3%, about 2%, or about 1% oxidation at M354 of the heavy chain sequence. As shown by the data presented herein, H₂O₂ forced degradation producing up to 16.7% oxidation at M354 gives 94% potency in bioassay (i.e. within assay variability—full function) and photolysis forced degradation up to 10.9% oxidation at M354 gives 90% potency in bioassay (i.e. within assay variability—full function). It is therefore expected that oxidation at M354 can go higher than 16.7% without any impact to relative potency or FcRn binding.

In one embodiment, the composition comprises ≤65% oxidation at M424 of the heavy chain sequence. In one embodiment, the composition comprises ≤65%, ≤60%, ≤50%, ≤40%, ≤30%, ≤20%, ≤15%, ≤10%, ≤5%, ≤2%, or ≤1% oxidation at M424 of the heavy chain sequence. In one embodiment, the composition comprises 0.01-65%, 0.01-60%, 0.01-50%, 0.01-40%, 0.01-30%, 0.01-20%, 0.01-15%, 0.01-10%, 0.01-5%, 0.01-4%, 0.01-3%, 0.01-2% or 0.01-1% oxidation at M424 of the heavy chain sequence. Alternatively, the composition comprises 0-65%, 0-60%, 0-50%, 0-40%, 0-30%, 0-20%, 0-15%, 0-10%, 0-5%, 0-4%, or 0-3% oxidation at M424 of the heavy chain sequence. Alternatively, the composition comprises 0.1% or more and 65% or less oxidation at M424 of the heavy chain sequence. Alternatively, the composition comprises about 10%, about 5%, about 4%, about 3%, about 2%, or about 1% oxidation at M424 of the heavy chain sequence. As shown by the data presented herein, H₂O₂ forced degradation producing up to 29.0% oxidation at M424 gives 94% potency in bioassay (i.e. within assay variability—full function) and photolysis forced degradation up to 27.0% oxidation at M424 gives 90% potency in bioassay (i.e. within assay variability—full function). It is therefore expected that oxidation at M424 can go higher than 29.0% without any impact to relative potency or FcRn binding.

In one embodiment, the composition comprises an oxidized variant of dostarlimab, wherein the oxidized variant comprises a heavy chain sequence of SEQ ID NO: 9 and a light chain sequence of SEQ ID NO: 10; wherein the composition comprises ≤65% oxidation at M248 and/or M354 and/or M424 of the heavy chain sequence.

In one aspect, the composition comprises an anti-PD-1 antibody comprising a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises: ≤100% acidic variant; and/or ≤35% basic variant; and/or ≥1% main isoform; and/or ≤65% oxidized variant.

In another aspect, the composition comprises an anti-PD-1 antibody comprising a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises: 5-60% acidic variant; and/or 0.1-35% basic variant; and/or 20-90% main isoform; and/or ≤65% oxidized variant.

In one example, oxidation can be determined using tryptic peptide mapping tandem mass spectrometry (peptide mapping LC-MS/MS). In one example, a sample comprising a composition described herein may be denatured with guanidine hydrochloride, reduced with dithiothreitol (DTT), alkylated with iodoacetamide, and digested with endoproteinase Lys-C (Lys-C) or trypsin. Enzymatic digestion with either Lys-C or trypsin can be accomplished at 37° C. for 4 hours. The sample digestion can be quenched with trifluoroacetic acid prior to the liquid chromatography with tandem mass spectrometry (LC-MS/MS) analysis. The LC-MS/MS analysis system may employ reverse-phase ultra-high performance liquid chromatography (UHPLC) with a C18 column, UV detection at 214 nm, and electrospray ionization mass spectrometry (ESI-MS). The peptides can then be detected with a UV detector and a mass spectrometer, (e.g. Thermo Scientific LTQ Orbitrap XL). The extracted ion chromatograms of the unmodified and modified peptides are used to calculate the levels of oxidation by dividing the area under the curve of the modified peptide by the total areas under the curve for both modified and unmodified peptides.

In one aspect, the composition comprises antibodies that are aggregated antibodies (High molecular weight (HMW) species) also referred to herein as an “aggregated variant”. The aggregated antibodies may comprise dimers or higher order structures formed of antibody monomers and subunits thereof. High molecular weight (HMW) species may therefore be comprised of dimerized antibodies and monomers with additional subunits (such as a monomer with two light chain subunits, or an LC-LC dimer that is non-covalently bound to the monomer). Aggregated variants can be, for example, covalent or non-covalent, reducible or non-reducible, and visible or subvisible aggregates of an antibody disclosed herein. Aggregated or fragmented variants can be characterized and distinguished from an antibody based on their size. For example, the size distribution of an antibody composition can be detected using size exclusion chromatography (SEC), such as SE-HPLC. In one aspect, the composition comprises an anti-PD-1 antibody having a heavy chain sequence comprising a CDRH1 comprising the amino acid sequence of SEQ ID NO: 1, a CDRH2 comprising the amino acid sequence of SEQ ID NO: 2, and a CDRH3 comprising the amino acid sequence of SEQ ID NO: 3, and a light chain sequence comprising a CDRL1 comprising the amino acid sequence of SEQ ID NO: 4, a CDRL2 comprising the amino acid sequence of SEQ ID NO: 5, and a CDRL3 comprising the amino acid sequence of SEQ ID NO: 6; wherein the composition comprises ≤36% aggregated variant. It will be understood that these aggregated variant embodiments may be combined with any of the antibody variants described herein.

In one aspect, the composition comprises an aggregated variant of an anti-PD-1 antibody, wherein the aggregated variant comprises a heavy chain sequence comprising a CDRH1 of SEQ ID NO: 1, a CDRH2 of SEQ ID NO: 2, and a CDRH3 of SEQ ID NO: 3, and a light chain sequence comprising a CDRL1 of SEQ ID NO: 4, a CDRL2 of SEQ ID NO: 5, and a CDRL3 of SEQ ID NO: 6; wherein the composition comprises ≤36% aggregated variant.

In one aspect, the composition comprises a population of anti-PD-1 antibodies that includes:

antibodies having a heavy chain amino acid sequence comprising SEQ ID NO: 1 (CDRH1), SEQ ID NO: 2 (CDRH2), and SEQ ID NO: 3 (CDRH3) and a light chain amino acid sequence comprising SEQ ID NO: 4 (CDRL1), SEQ ID NO: 5 (CDRL2) and SEQ ID NO: 6 (CDRL3), and

aggregated variants thereof, wherein ≤36% of the population of antibodies is comprised of the aggregated variants.

In one embodiment, the antibody comprises a heavy chain variable region at least about 90% identical to the amino acid sequence of SEQ ID NO: 7 and/or a light chain variable region at least about 90% identical to the amino acid sequence of SEQ ID NO: 8. In a further embodiment, the antibody is at least about 90% identical to the heavy chain amino acid sequence of SEQ ID NO: 9 and/or at least about 90% identical to the light chain amino acid sequence of SEQ ID NO: 10. In a yet further embodiment, the antibody comprises a heavy chain sequence of SEQ ID NO: 9 and a light chain sequence of SEQ ID NO: 10.

In one aspect, the composition comprises an anti-PD-1 antibody comprising a heavy chain variable region at least about 90% identical to the amino acid sequence of SEQ ID NO: 7 and/or a light chain variable region at least about 90% identical to the amino acid sequence of SEQ ID NO: 8, wherein the composition comprises ≤36% aggregated variant. In a further aspect, the composition comprises an anti-PD-1 antibody comprising a heavy chain variable region of SEQ ID NO: 7 and/or a light chain variable region of SEQ ID NO: 8, wherein the composition comprises ≤36% aggregated variant.

In one aspect, the composition comprises a population of anti-PD-1 antibodies that includes:

antibodies having a heavy chain variable region as set forth in SEQ ID NO: 7 and a light chain variable region as set forth in SEQ ID NO: 8, and

aggregated variants thereof, wherein ≤36% of the population of antibodies is comprised of the aggregated variants.

In one aspect, the composition comprises an anti-PD-1 antibody comprising a heavy chain sequence at least about 90% identical to the amino acid sequence of SEQ ID NO: 9 and/or a light chain sequence at least about 90% identical to the amino acid sequence of SEQ ID NO: 10, wherein the composition comprises ≤36% aggregated variant.

In one aspect, the composition comprises an antibody having a heavy chain sequence of SEQ ID NO: 9 and a light chain sequence of SEQ ID NO: 10, and an antibody having a heavy chain sequence at least about 90% identical to the amino acid sequence of SEQ ID NO: 9 and/or a light chain sequence at least about 90% identical to the amino acid sequence of SEQ ID NO: 10, wherein the composition comprises ≤36% aggregated variant.

In one aspect, the composition comprises an anti-PD-1 antibody comprising a heavy chain sequence of SEQ ID NO: 9 and a light chain sequence of SEQ ID NO: 10, wherein the composition comprises ≤36% aggregated variant. In one embodiment, the composition comprises an aggregated variant of dostarlimab, wherein the aggregated variant comprises a heavy chain sequence of SEQ ID NO: 9 and a light chain sequence of SEQ ID NO: 10; wherein the composition comprises ≤36% aggregated variant. In one embodiment, the composition comprises an aggregated variant of dostarlimab, wherein the aggregated variant comprises a heavy chain sequence of SEQ ID NO: 9 and a light chain sequence of SEQ ID NO: 10; wherein the composition comprises aggregated variant in the range of 0.01% and 36% aggregated variant.

In one aspect, the composition comprises a population of anti-PD-1 antibodies that includes:

antibodies having a heavy chain amino acid sequence as set forth in SEQ ID NO: 9 and a light chain amino acid sequence as set forth in SEQ ID NO: 10, and
aggregated variants thereof, wherein ≤36% of the population of antibodies is comprised of the aggregated variants.

The antibody composition may comprise ≤36% aggregated variants, such as ≤35%, ≤30%, ≤26%, ≤25%, ≤20%, ≤10%, ≤5%, ≤4%, ≤3%, ≤2%, or ≤1% aggregated variants. In another embodiment, the composition may comprise 0.01-36%, 0.01-35%, 0.01-30%, 0.01-26%, 0.01-25%, 0.01-20%, 0.01-10%, 0.01-5%, 0.01-4%, 0.01-3%, 0.01-2%, or 0.01-1% aggregated variants. Alternatively, the composition comprises more than 1% and less than 36% aggregated variants. Alternatively, the composition may comprise about 10%, about 5%, about 4%, about 3%, about 2%, or about 1% aggregated variants. As shown by the data presented herein, thermal forced degradation producing up to 11.2% of the composition comprising of aggregated antibodies gives 86% potency in bioassay (i.e. within assay variability—full function). Acid treated samples producing up to 15.2% of the composition comprising of aggregated antibodies gives 125% potency in bioassay or 88% potency in MSD (i.e. within assay variability—full function). Extrapolating from thermal forced degradation data for aggregated variant up to 36% can result in at least 60% potency and up to 26% can result in at least 70% potency. This is calculated using a linear slope for 0.9% (control), 1.4% (40° C. at 3 weeks) and 11.2% (50° C. at 3 weeks) aggregated variant thermal treated samples, which have 98%, 94% and 86% potency (bioassay), respectively.

In one aspect, the composition comprises an anti-PD-1 antibody comprising a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises: ≤100% acidic variant; and/or ≤35% basic variant; and/or ≥1% main isoform; and/or ≤65% oxidized variant; and/or ≤36% aggregated variant.

In another aspect, the composition comprises an anti-PD-1 antibody comprising a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises: 5-60% acidic variant; and/or 0.1-35% basic variant; and/or 20-90% main isoform; and/or ≤65% oxidized variant; and/or ≤36% aggregated variant.

Fragmented variants (“fragment variant”) are variants which comprise a portion of a full length antibody. For example, such fragments include Fab, Fab′, F(ab′)2, and Fv fragments, diabodies, linear antibodies, single-chain antibody molecules and immunoglobulin single variable domains. The antibody composition may comprise ≤10% fragmented antibodies, such as ≤5%, ≤4.6%, ≤4.5%, ≤4.4%, ≤4.3%, ≤4.2%, ≤4.1%, ≤4%, ≤3.5%, ≤3%, ≤2.5%, ≤2%, ≤1.5%, ≤1%, ≤0.5% or ≤0.05% fragmented antibodies. In another embodiment, the composition may comprise 0.01-10%, 0.01-5%, 0.01-4.6%, 0.01-4.5%, 0.01-4%, 0.01-3.5%, 0.01-3%, 0.01-2.5%, 0.01-2%, 0.01-1.5%, 0.01-1%, 0.01-0.5%, 0.01-0.1%, or 0.01-0.05% fragmented antibodies. In another embodiment, the composition may comprise 0.5-10%, 0.5-5%, 0.5-4.6%, 0.5-4.5%, 0.5-4%, 0.5-3.5%, 0.5-3%, 0.5-2.5%, 0.5-2%, 0.5-1.5%, 0.5-1%, 0.6-1.5%, or 0.6-1.0% fragmented antibodies. Alternatively, the composition may comprise about 10%, about 5%, about 4%, about 3%, about 2%, about 1%, or about 0.5% fragmented antibodies. It will be understood that these fragmented variant embodiments may be combined with any one of the antibody variants described herein.

Deamidation, which may, for example, occur during production and/or storage, may be an enzymatic reaction or a chemical reaction. Deamidation may occur via simple chemical reaction through intramolecular cyclisation where the amide nitrogen of the next amino acid in the chain nucleophilically attacks the amide (N+1 attacks N); forming a succinimide intermediate. Deamidation may primarily convert asparagine (N) to iso-aspartic acid (iso-aspartate) and aspartic acid (aspartate) (D) at an approximately 3:1 ratio. This deamidation reaction may therefore be related to isomerization of aspartate (D) to iso-aspartate. The deamidation of asparagine and the isomerization of aspartate, both may involve the intermediate succinimide. To a much lesser degree, deamidation can occur with glutamine residues in a similar manner. Deamidation can occur in a CDR, in a Fab (non-CDR region), or in an Fc region. Isomerization is the conversion of aspartate (D) to iso-aspartate which involves the intermediate succinimide (succinimide-aspartic acid residue).

In one aspect, a composition comprises an antibody comprising a deamidation post-translational modification (“deamidation” or “deamidated”) also referred to herein as a “deamidated variant”.

In one embodiment, the antibody comprises deamidation of an asparagine residue in a CDR of the heavy chain sequence and/or a CDR of the light chain sequence. In a further embodiment, the antibody comprises deamidation of an asparagine residue in a CDR of the heavy chain sequence. In one embodiment, the antibody comprises deamidation of an asparagine residue in the Fc region of the heavy chain sequence and/or the Fc region of the light chain sequence. The deamidated variant may be present in one or both chains of the heavy chain or light chain. It will be understood that these deamidated variant embodiments may be combined with any one of the antibody variants described herein. In some embodiments, the deamidated variant comprises one or a combination of deamidation at: N380 and/or N385 of the Fc region of the heavy chain sequence.

In one embodiment, the deamidated variant comprises a deamidated residue selected from: an aspartic acid residue, a succinimide-aspartic acid residue, or an iso aspartic acid residue.

In one aspect, the composition comprises an antibody comprising a sequence that is at least about 90% identical to the heavy chain amino acid sequence of SEQ ID NO: 9 (and optionally comprising a sequence that is at least about 90% identical to the light chain sequence of SEQ ID NO: 10), and comprises deamidation in the heavy chain sequence, e.g. deamidation at amino acid residue N380 and/or N385 of the Fc region. In some embodiments, the deamidated variant comprises up to 100% deamidation at N380 and/or N385 of SEQ ID NO: 9.

Deamidation may result in a sequence change where an asparagine residue (N) is converted to an aspartic acid residue (D). Therefore, in one embodiment, the deamidated variant comprises a heavy chain sequence of SEQ ID NO: 11 (i.e. the heavy chain sequence with N380D). In another embodiment, the deamidated variant comprises a heavy chain sequence of SEQ ID NO: 12 (i.e. the heavy chain sequence with N385D). In a yet further alternative embodiment, the deamidated variant comprises a heavy chain sequence of SEQ ID NO: 13 (i.e. the heavy chain sequence with N380D and N385D).

The composition may comprise up to 100% deamidated variant. In one aspect, the composition comprises an antibody having a heavy chain sequence of SEQ ID NO: 9 and a light chain sequence of SEQ ID NO: 10, wherein the composition comprises up to 100% deamidated variant.

In one embodiment, the composition comprises up to 100% deamidation at N380 and/or N385 of the heavy chain sequence. In one embodiment, the composition comprises 0-100%, 0-90%, 0-80%, 0-70%, 0-60%, 0-50%, 0-40%, 0-30%, 0-20%, or 0-10% deamidation at N380. Alternatively, the composition comprises 0.1-100%, 0.1-90%, 0.1-80%, 0.1-70%, 0.1-60%, 0.1-50%, 0.1-40%, 0.1-30%, 0.1-20%, or 0.1-10% deamidation at N380. Alternatively, the composition comprises 1-100%, 1-90%, 1-80%, 1-70%, 1-60%, 1-50%, 1-40%, 1-30%, 1-20%, or 1-10% deamidation at N380. Alternatively, the composition comprises 2-100%, 3-100%, 4-100%, 5-100%, 6-100%, 7-100%, 8-100%, 9-100%, 2-30%, 3-30%, 4-30%, 5-30%, 2-40%, 3-40%, 4-40%, 5-40%, 2-10%, 3-10%, 4-10%, or 5-9% deamidation at N380. Alternatively, the composition comprises 1% or more, 2% or more, 3% or more, 4% or more, 5% or more, 6% or more, 7% or more, 8% or more, 9% or more, or 10% or more deamidation at N380. As shown in the data presented herein, base treated samples producing up to 27.8% deamidation at N380 gives 96% potency in bioassay (i.e. within assay variability—full function), therefore it is expected that deamidation at N380 can go higher than the reported level of 27.8% without any impact to relative potency or FcRn binding.

In one embodiment, the composition comprises 0-100%, 0-90%, 0-80%, 0-70%, 0-60%, 0-50%, 0-40%, 0-30%, 0-20%, or 0-10% deamidation at N385. Alternatively, the composition comprises 0.1-100%, 0.1-90%, 0.1-80%, 0.1-70%, 0.1-60%, 0.1-50%, 0.1-40%, 0.1-30%, 0.1-20%, or 0.1-10% deamidation at N385. Alternatively, the composition comprises 1-100%, 1-90%, 1-80%, 1-70%, 1-60%, 1-50%, 1-40%, 1-30%, 1-20%, or 1-10% deamidation at N385. Alternatively, the composition comprises 0.5% or more, 1% or more, or 2% or more deamidation at N385. As shown in the data presented herein, base treated samples producing up to 27.2% deamidation at N385 gives 96% potency in bioassay (i.e. within assay variability—full function), therefore it is expected that deamidation at N385 can go higher than the reported level of 27.2% without any impact to relative potency or FcRn binding.

In one aspect, the composition comprises an antibody having a heavy chain sequence of SEQ ID NO: 9 and a light chain sequence of SEQ ID NO: 10, wherein the composition comprises up to 100% deamidation at N380 and/or N385 of the heavy chain.

In some embodiments, the composition comprises about 0.5-2%, about 0.5%, about 1%, about 1.5% or about 2% deamidation at N84 of SEQ ID NO: 9. In some embodiments, the composition comprises about 0.5-2%, about 0.5%, about 1%, about 1.5% or about 2% deamidation at N137 of SEQ ID NO: 9. In some embodiments, the composition comprises about 5-8%, about 5%, about 6%, about 7%, or about 8% deamidation at N311 of SEQ ID NO: 9. In some embodiments, the composition comprises about 0.5-3%, about 0.5%, about 1%, about 1.5%, about 2%, about 2.5% or about 3% deamidation at N430 of SEQ ID NO: 9.

In one example, deamidation can be determined using Lys-C and/or tryptic peptide mapping tandem mass spectrometry (peptide mapping LC-MS/MS) as described hereinbefore.

In one aspect, a composition comprises an antibody comprising an isomerization post-translational modification (“isomerization” or “isomerized”), also referred to herein as an “isomerized variant”. The variant may comprise an isomerized amino acid residue in the heavy chain sequence and/or the light chain sequence, such as a CDR of the heavy chain sequence and/or a CDR of the light chain sequence. The isomerized variant may be present in one or both chains of the heavy chain and/or light chain. An isomerization post-translational modification may result in iso-aspartic acid and/or succinimide-aspartic acid residue. In one example, aspartic acid (Asp) isomerization can be determined using Lys-C and/or tryptic peptide mapping tandem mass spectrometry (peptide mapping LC-MS/MS) as described hereinbefore. It will be understood that these isomerized variant embodiments may be combined with any one of the antibody variants described herein.

In one aspect, the composition comprises an antibody having a heavy chain sequence of SEQ ID NO: 9 and a light chain sequence of SEQ ID NO: 10, wherein the composition comprises up to 100% isomerized variant. In one embodiment, the composition comprises an isomerized variant of dostarlimab, wherein the isomerized variant comprises a heavy chain sequence of SEQ ID NO: 9 and a light chain sequence of SEQ ID NO: 10; wherein the composition comprises ≤100% isomerized variant.

In some embodiments, the composition comprises an isomerized variant. In some embodiments, the composition comprises up to 100% isomerized variant. The composition may comprise 0-100%, 0-90%, 0-80%, 0-70%, 0-60%, 0-50%, 0-40%, 0-30%, 0-15%, 0-20%, or 0-10% isomerized variant. Alternatively, the composition may comprise 0.1-100%, 0.1-90%, 0.1-80%, 0.1-70%, 0.1-60%, 0.1-50%, 0.1-40%, 0.1-30%, 0.1-20%, 0.1-15% or 0.1-10% isomerized variant. Alternatively, the composition may comprise 1-100%, 1-90%, 1-80%, 1-70%, 1-60%, 1-50%, 1-40%, 1-30%, 1-20%, 1-15% or 1-10% isomerized variant.

In one embodiment, the composition comprises up to 100% isomerization at D147 of SEQ ID NO: 9. The composition may comprise 0-100%, 0-90%, 0-80%, 0-70%, 0-60%, 0-50%, 0-40%, 0-30%, 0-20%, 0-15% or 0-10% isomerization at D147 of the heavy chain sequence. Alternatively, the composition may comprise 0.1-100%, 0.1-90%, 0.1-80%, 0.1-70%, 0.1-60%, 0.1-50%, 0.1-40%, 0.1-30%, 0.1-20%, 0-15% or 0.1-10% isomerization at D147 of the heavy chain sequence. In some embodiments, the composition comprises 1% or more isomerization at D147 of the heavy chain sequence. As shown in the data presented herein, base treated samples producing up to 20.8% isomerization at D147 gives 96% potency in bioassay (i.e. within assay variability—full function), therefore it is expected that isomerization at D147 can go higher than the reported level of 20.8% without any impact to relative potency or FcRn binding.

In one aspect, the composition comprises an antibody having a heavy chain sequence of SEQ ID NO: 9 and a light chain sequence of SEQ ID NO: 10, wherein the composition comprises up to 100% isomerization at D147 of the heavy chain.

In some embodiments, the composition comprises 0-15%, 0.1-15%, 1-15%, 1% or more, 1.5% or more, or 2% or more isomerization at D151, D167, D261, D266, D276, D395 D397 and/or 409 of SEQ ID NO: 9. For example, the composition comprises about 2.3% isomerization at D62 of SEQ ID NO: 9. For example, the composition comprises about 13.1% isomerization at D261/266/276 of SEQ ID NO: 9. For example, the composition comprises about 3.1% isomerization at D151/167 of SEQ ID NO: 9. For example, the composition comprises about 2.7% isomerization at D395/397/409 of SEQ ID NO: 9.

The antibody composition may comprise (i) the antibody (as described herein, e.g. an antibody comprising a heavy chain amino acid sequence of SEQ ID NO: 9 and a light chain amino acid sequence of SEQ ID NO: 10); and (ii) antibody variants that include one or more or a combination of: amino acid sequence variants (e.g. deamidated or C-terminal lysine clipped variants), oxidized variants, isomerized variants, aggregated variants, and/or fragmented variants.

Therefore, in one aspect, there is provided a composition comprising an antibody having a heavy chain sequence of SEQ ID NO: 9 and a light chain sequence of SEQ ID NO: 10, wherein the composition comprises: (i) ≤65% oxidized variant; and (ii) ≤36% aggregated variant.

In one aspect, a composition comprises an antibody comprising a heavy chain sequence having one or a combination of sequences selected from SEQ ID NO: 9, SEQ ID NO: 11, SEQ ID NO: 12 and/or SEQ ID NO: 13, and a light chain sequence of SEQ ID NO: 10, wherein the composition comprises ≤65% oxidized variant.

In one aspect, a composition comprises an antibody comprising a heavy chain sequence having one or a combination of sequences selected from SEQ ID NO: 9, SEQ ID NO: 11, SEQ ID NO: 12 and/or SEQ ID NO: 13, and a light chain sequence of SEQ ID NO: 10, wherein the composition comprises ≤36% aggregated variant.

In another embodiment a composition comprising a variant has at least 60%, at least 65%, at least 70%, at least 75%, at least 80%, at least 85%, at least 90%, at least 95%, at least 96%, at least 97%, at least 98%, or at least 99% of the potency of the reference standard which has 100% potency. In one aspect, a composition comprises a variant of an anti-PD-1 antibody, wherein the variant comprises a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition has at least 60% of the potency of a composition comprising a heavy chain sequence of SEQ ID NO: 9 and a light chain sequence of SEQ ID NO: 10, 10-97% acidic variant, 0.1-35% basic variant, 2-80% main isoform, 4.8% or less LC W50 oxidized variant, 1% or less HC M34 oxidized variant, 1.2% or less HC M103 oxidized variant, 15.2% or less aggregated variant, 16.7% or less HC M354 oxidized variant, 29.0% or less HC M424 oxidized variant, 47.1% or less HC M248 oxidized variant, 20.8% or less HC D147 isomerized variant, 13.1% or less HC D151 or D167 isomerized variant, 3.1% or less HC D261, D266 or D276 isomerization variant, 4.6% or less fragmented variants, 27.8% or less HC N380 deamidated variant, 27.2% or less HC N385 deamidated variant, about 7.4% or less HC N311 deamidated variant, about 2.0% or less N430 deamidated variant, 90% or more heavy chain (HC) C-terminal lysine deleted variants (ΔK443), and 1% or less HC N-terminal pyro-glutamate variant. Potency can be determined using the bioassay as described herein.

Glycation is a post-translational modification comprising a non-enzymatic chemical reaction between a reducing sugar, such as glucose, and a free amine group in the protein, and is typically observed at the epsilon amine of lysine side chains or at the N-terminus of the protein. Glycation can occur during production and/or storage in the presence of reducing sugars.

Disulfide bond scrambling can occur during production and/or storage conditions. Under certain circumstances, disulfide bonds may break or form incorrectly, resulting in unpaired cysteine residues (—SH). These free (unpaired) sulfhydryls (—SH) may promote shuffling.

The formation of a thioether and racemization of a disulfide bond can occur under basic conditions, in production or storage, through a beta elimination of disulfide bridges back to cysteine residues via a dehydroalanine and persulfide intermediate. Subsequent crosslinking of dehydroalanine and cysteine may result in the formation of a thioether bond or the free cysteine residues may reform a disulfide bond with a mixture of D- and L-cysteine.

Trisulfides may result from insertion of a sulfur atom into a disulfide bond (Cys-SS—S-Cys) and may be formed due to the presence of hydrogen sulfide in production cell culture.

N-terminal glutamine (Q, Gln) and glutamate (glutamic acid) (E, Glu) in the heavy chain and/or light chain may form pyroglutamate (pGlu) via cyclization. pGlu formation may form in the production bioreactor, but it can also be formed, for example, non-enzymatically, depending on pH and temperature of processing and storage conditions. Cyclization of N-terminal Q or E is commonly observed in natural human antibodies.

C-terminal lysine clipping (also referred to as C-terminal lysine cleavage) is an enzymatic reaction catalyzed by carboxypeptidases, and is commonly observed in recombinant and natural human antibodies. Variants of this process include removal of lysine from one or both heavy chains due to cellular enzymes from the recombinant host cell. Administration to the human subject/patient is likely to result in the removal of any remaining C-terminal lysine.

In one embodiment, a post-translational modification is an antibody variant (e.g. a sequence variant). Exemplary post-translational modification antibody variants comprise an asparagine (N, Asn) to aspartic acid (D, Asp) switch (“deamidation”), an N-terminal pyro-glutamate, and/or a C-terminal lysine cleavage. In one example, antibody variants, e.g. N380D or N385D in the heavy chain sequence, can be determined using Lys-C and/or tryptic peptide mapping tandem mass spectrometry (peptide mapping LC-MS/MS) as described hereinbefore. The extracted ion chromatograms of the unmodified and modified peptides are used to calculate the levels of antibody variant, e.g. N380D or N385D, by dividing the area under the curve of the modified peptide by the total areas under the curve for both modified and unmodified peptides.

In one aspect, a composition comprises an antibody comprising an N-terminal pyroglutamic acid (“pyroglutamic acid”) post-transitional modification (“N-terminal pyro-glutamate variant”) in the heavy chain amino acid sequence. In one embodiment, the composition comprises an antibody comprising a sequence that is at least about 90% identical to the heavy chain amino acid sequence of SEQ ID NO: 9 (and optionally comprising a sequence that is at least about 90% identical to the light chain sequence of SEQ ID NO: 10), and comprises pyroglutamic acid at the N-terminus of the heavy chain.

In one embodiment, the composition comprises up to 100% heavy chain N-terminal pyro-glutamate variant. The composition may comprise 0-100%, 0-90%, 0-80%, 0-70%, 0-60%, 0-50%, 0-40%, 0-30%, 0-20%, or 0-10% of the heavy chain N-terminal pyro-glutamate variant. Alternatively, the composition may comprise 0.1-100%, 0.1-90%, 0.1-80%, 0.1-70%, 0.1-60%, 0.1-50%, 0.1-40%, 0.1-30%, 0.1-20%, or 0.1-10% of the heavy chain N-terminal pyro-glutamate variant. Alternatively, the composition may comprise 1-100%, 1-90%, 1-80%, 1-70%, 1-60%, 1-50%, 1-40%, 1-30%, 1-20%, or 1-10% of the heavy chain N-terminal pyro-glutamate variant. Alternatively, the composition may comprise ≤10%, ≤9%, ≤8%, ≤7%, ≤6%, ≤5%, ≤2% or ≤1% heavy chain N-terminal pyro-glutamate variant.

In one aspect, a composition comprises an antibody comprising deletion of the C-terminal lysine (K443) in the heavy chain amino acid sequence. In one embodiment, the composition comprises an antibody comprising a sequence that is at least about 90% identical to the heavy chain amino acid sequence of SEQ ID NO: 9 (and optionally comprising a sequence that is at least about 90% identical to the light chain sequence of SEQ ID NO: 10), and comprises deletion of the lysine residue (K443) at the C-terminus of the heavy chain.

In one embodiment, the composition comprises up to 100% heavy chain C-terminal lysine cleaved variant. In another embodiment, the composition comprises 10% or more heavy chain C-terminal lysine cleaved variant. In another embodiment, the composition comprises ≥10%, ≥20%, ≥30%, ≥40%, ≥50%, 60%, ≥70%, ≥80%, ≥90% or ≥95% heavy chain C-terminal lysine cleaved variant. The composition may comprise 1-100%, 10-100%, 20-100%, 30-100%, 40-100%, 50-100%, 60-100%, 70-100%, 80-100% or 90-100% heavy chain C-terminal lysine cleaved variant. Alternatively, the composition may comprise about 50%, about 60%, about 70%, about 80%, about 90%, about 95%, about 95-99%, about 96-99% or about 97-99% heavy chain C-terminal lysine cleaved variant.

In one embodiment, the composition comprises up to 100% heavy chain N-terminal pyro-glutamate variant and up to 100% heavy chain C-terminal lysine cleaved variant.

In one aspect, the composition comprises an antibody having a heavy chain sequence of SEQ ID NO: 9 and a light chain sequence of SEQ ID NO: 10, wherein the composition comprises up to 100% heavy chain N-terminal pyro-glutamate variant, and/or up to 100% heavy chain C-terminal lysine cleaved variant.

In one example, N-terminal pyroglutamic acid and C-terminal lysine cleavage can be determined using Lys-C and/or tryptic peptide mapping tandem mass spectrometry (peptide mapping LC-MS/MS) as described hereinbefore.

The binding of Neonatal Fc Receptor (FcRn) to an anti-PD-1 antibody can measured using surface plasmon resonance (SPR). The antibody can be captured by FcRn, which is immobilized on a nitrilotriacetic acid (NTA) sensor chip. The FcRn binding concentration of the sample can be determined by interpolation of the binding response on a calibration curve. Specific binding activity (%) is calculated by dividing the FcRn binding concentration by the total protein concentration.

The antibody composition comprising the antibody and antibody variants described above retain specific antigen binding and/or FcRn binding and/or potency. For example, the antibody composition comprising the antibody and antibody variants and post-translational modification variants described above has >0.70 PD-1 specific antigen binding; and/or >70% FcRn binding and/or >70% potency. Thus these levels (%) of variants can be tolerated in the antibody composition without significantly impacting function (i.e. without resulting in reduced activity). In one embodiment, “reduced function” or “reduced activity” means that binding to PD-1, or binding to FcRn, or potency is reduced as a percentage compared to a reference standard, and is significant over assay variability. For example, reduced function or activity or potency can be described as a reduction of ≥5%, ≥10%, ≥15%, ≥20%, ≥25%, ≥30%, ≥35%, ≥40%, ≥45%, or ≥50%.

For example, the reference standard (or reference material or control or unstressed control) is a composition comprising a heavy chain sequence of SEQ ID NO: 9 and a light chain sequence of SEQ ID NO: 10. In one aspect, the reference standard comprises 10-97% acidic variant, and/or 0.1-35% basic variant, and/or 2-80% main isoform. In an embodiment, the reference standard comprises 4.8% or less LC W50 oxidized variant. In another embodiment, the reference standard comprises 1% or less HC M34 oxidized variant. In another embodiment, the reference standard comprises 1.2% or less HC M103 oxidized variant. In another embodiment, the reference standard comprises 10-97% acidic variant, and/or 0.1-35% basic variant, and/or 2-80% main isoform, and/or 4.8% or less LC W50 oxidized variant, and/or 1% or less HC M34 oxidized variant, and/or 1.2% or less HC M103 oxidized variant. In another embodiment, the reference standard comprises 15.2% or less aggregated variant. In another embodiment, the reference standard comprises a heavy chain sequence of SEQ ID NO: 9 and a light chain sequence of SEQ ID NO: 10, 10-97% acidic variant, and/or 0.1-35% basic variant, and/or 2-80% main isoform, and/or 4.8% or less LC W50 oxidized variant, and/or 1% or less HC M34 oxidized variant, and/or 1.2% or less HC M103 oxidized variant, and/or 15.2% or less aggregated variant.

In another embodiment, the reference standard further comprises 16.7% or less HC M354 oxidized variant. In another embodiment, the reference standard further comprises 29.0% or less M424 oxidized variant. In another embodiment, the reference standard further comprises 47.1% or less HC M248 oxidized variant. In another embodiment, the reference standard further comprises 20.8% or less HC D147 isomerized variant. In another embodiment, the reference standard further comprises 13.1% or less HC D151 or D167 isomerized variant. In another embodiment, the reference standard further comprises 3.1% or less HC D261, D266 or D276 isomerization variant. In another embodiment, the reference standard further comprises 4.6% or less fragmented variant. In another embodiment, the reference standard further comprises 27.8% or less HC N380 deamidated variant and/or 27.2% or less HC N385 deamidated variant. In a further embodiment, the reference standard further comprises about 7.4% or less HC N311 deamidated variant and/or about 2.0% or less N430 deamidated variant. In another embodiment, the reference standard further comprises 90% or more heavy chain (HC) C-terminal lysine deleted variants (ΔK443), and 1% or less HC N-terminal pyro-glutamate variant. In another embodiment, the reference standard comprises a heavy chain sequence of SEQ ID NO: 9 and a light chain sequence of SEQ ID NO: 10, 10-97% acidic variant, and/or 0.1-35% basic variant, and/or 2-80% main isoform, and/or 4.81% or less LC W50 oxidized variant, and/or 1% or less HC M34 oxidized variant, and/or 1.2% or less HC M103 oxidized variant, and/or 15.2% or less aggregated variant, 16.7% or less HC M354 oxidized variant, and/or 29.0% or less HC M424 oxidized variant, and/or 47.1% or less HC M248 oxidized variant, and/or 20.8% or less HC D147 isomerized variant, and/or 13.1% or less HC D151 or D167 isomerized variant, and/or 3.1% or less HC D261, D266 or D276 isomerization variant, and/or 4.6% or less fragmented variants, and/or 27.8% or less HC N380 deamidated variant, and/or 27.2% or less HC N385 deamidated variant, and/or about 7.4% or less HC N311 deamidated variant, and/or about 2.0% or less N430 deamidated variant, and/or 90% or more heavy chain (HC) C-terminal lysine deleted variants (ΔK443), and/or 1% or less HC N-terminal pyro-glutamate variant.

In one embodiment, the reference standard comprises a heavy chain sequence of SEQ ID NO: 9 and a light chain sequence of SEQ ID NO: 10, 10-30% acidic variant; and/or 0.1-10% basic variant; and/or 60-80% main isoform, and/or about 1% or less LC W50 oxidized variant, and/or about 1% or less HC M34 oxidized variant, and/or about 1% or less HC M103 oxidized variant, and/or about 1% aggregated variant, about 1% or less HC M354 oxidized variant, and/or about 1% or less HC M424 oxidized variant, and/or about 2-3% HC M248 oxidized variant, and/or about 1% or less HC D147 isomerized variant, and/or about 1% HC D151 or D167 isomerized variant, and/or about 0.6-1% fragmented variants, and/or 5-9% HC N380 deamidated variant, and/or about 1% or less HC N385 deamidated variant, and/or about 5.8% HC N311 deamidated variant, and/or about 1.2% N430 deamidated variant, and/or about 97-99% heavy chain (HC) C-terminal lysine deleted variants (ΔK443), and/or about 1% or less HC N-terminal pyro-glutamate variant.

The reference standard as defined herein is a composition comprising an anti-PD-1 antibody.

The composition may comprise a mixture of antibody variants and post-translational modification variants. For example, the antibody composition may comprise two or more of acidic variants, basic variants, oxidation variants, deamidation variants, isomerized variants, aggregated variants, fragmented variants, N-terminal pyro-glutamate variants, and C-terminal lysine cleaved variants.

In one aspect, the composition comprises an antibody having a heavy chain sequence of SEQ ID NO: 9 and a light chain sequence of SEQ ID NO: 10, wherein the composition comprises any one or a combination of: (i) up to 100% acidic variant, (ii) up to 35% basic variant, (iii) ≤34% oxidation at W50 of the light chain, (iv) ≤21% oxidation at M34 of the heavy chain, (v) ≤64% oxidation at M103 of the heavy chain, (vi) ≤65% oxidation at M248, (vii) ≤65% oxidation at M354, (viiii) ≤65% oxidation at M424, and/or (ix) ≤36% aggregated variant.

In one aspect, the composition comprises an antibody having a heavy chain sequence of SEQ ID NO: 9 and a light chain sequence of SEQ ID NO: 10, wherein the composition comprises any one or a combination of: (i) up to 100% acidic variant, (ii) up to 35% basic variant, (iii) ≤64% oxidation at M103 of the heavy chain, (iv) ≤34% oxidation at W50 of the light chain, (v) ≤21% oxidation at M34 of the heavy chain, (vi) ≤65% oxidation at M248, (vii) ≤65% oxidation at M354, (viii) ≤65% oxidation at M424; (ix) ≤36% aggregated variant, (x) up to 100% isomerization at D147 of the heavy chain; (xi) up to 100% deamidation at N380, (xii) up to 100% deamidation at N385, (xiii) up to 100% heavy chain N-terminal pyro-glutamate variant, and/or (xiv) up to 100% heavy chain C-terminal lysine cleaved variant.

In one aspect, the composition comprises an antibody having a heavy chain sequence having one or a combination of sequences selected from SEQ ID NO: 9, SEQ ID NO: 11, SEQ ID NO: 12 and/or SEQ ID NO: 13, and a light chain sequence of SEQ ID NO: 10, wherein the composition comprises any one or a combination of: (i) up to 100% acidic variant, (ii) up to 35% basic variant, (iii) ≤34% oxidation at W50 of the light chain, (iv) ≤21% oxidation at M34 of the heavy chain, (v) ≤64% oxidation at M103 of the heavy chain, (vi) ≤65% oxidation at M248, (vii) ≤65% oxidation at M354, (viii) ≤65% oxidation at M424, and/or (ix) ≤36% aggregated variant.

The present invention encompasses antibodies which may have been subjected to, or have undergone, one or more of a post-translational modification described herein. Exemplary compositions may comprise a mixture or blend of antibodies: 1) with and without post-translational modifications (1 or more, or 2 or more) described herein. Therefore, the composition may comprise a population of antibodies with post-translational modifications and a population of antibodies without post-translational modifications.

The compositions described may have been subjected to, or have undergone, one or more post-translational modifications. The modification may occur in a CDR, the variable framework region, or the constant region. The modification may result in a change in charge of the molecule.

In one embodiment, a post-translational modification described herein, does not result in a significant change in antigen binding affinity, biological activity, pharmacokinetics (PK)/pharmacodynamics (PD), aggregation, immunogenicity, and/or binding to an Fc receptor, except where specified and described as a product-related impurity.

### Methods of Production

The antibodies and compositions described herein can be obtained by any means, including via in vitro sources (e.g. a hybridoma or a cell line producing an antibody recombinantly) and in vivo sources (e.g. rodents). Methods for generating antibodies are known in the art and are described, for example, in WO2018/085468.

For example, the compositions may be expressed in and purified from recombinant expression systems. In one embodiment, the composition is produced by a method of culturing a host cell under conditions suitable for expression of an antibody comprising SEQ ID NO: 9 and SEQ ID NO: 10, wherein the composition is expressed, and optionally purified, and optionally formulated within a pharmaceutical composition.

A number of different expression systems and purification regimes can be used to produce the compositions. Generally, host cells are transformed with a recombinant expression vector encoding the antibody. A wide range of host cells can be employed, including eukaryotic cell lines of mammalian origin (e.g. CHO, Perc6, HEK293, HeLa, NSO). Suitable host cells include mammalian cells such as Chinese Hamster Ovary CHO cells (e.g. CHOK1 and CHO-DG44). In one embodiment, the antibody is produced by a Chinese Hamster Ovary cell.

The host cell may be an isolated host cell. The host cell is usually not part of a multicellular organism (e.g. plant or animal). The host cell may be a non-human host cell.

Appropriate cloning and expression vectors for use with eukaryotic or mammalian cellular hosts and methods of cloning are known in the art.

The host cells are cultured to express the recombinant expression vector encoding the antibody.

The composition may be recovered and purified by conventional protein purification procedures. For example, the composition may be harvested directly from the culture medium. Harvest of the cell culture medium may be via clarification, for example by centrifugation and/or depth filtration. Recovery of the composition is followed by purification to ensure adequate purity. Therefore, in one aspect, there is provided a cell culture medium comprising the composition described herein. In one embodiment, the cell culture medium comprises CHO cells.

The composition may be subsequently purified from the cell culture medium. This may comprise harvesting the cell culture supernatant, placing the cell culture supernatant in contact with a purification medium (e.g. protein A resin or protein G resin to bind antibody molecules) and eluting the antibody molecules from the purification medium to produce an eluate. Therefore, in one aspect, there is provided an eluate comprising the composition described herein.

One or more chromatography steps may be used in purification, for example one or more chromatography resins; and/or one or more filtration steps. For example affinity chromatography using resins, such as protein A, G, or L may be used to purify the composition. Alternatively, or in addition to, an ion-exchange resin such as a cation-exchange may be used to purify the composition.

Alternatively the purification steps comprise: an affinity chromatography resin step, followed by a cation-exchange resin step.

### Pharmaceutical Compositions and Formulations

A composition described herein can be in the form of a pharmaceutical composition.

In one aspect there is provided a pharmaceutical composition comprising the composition and at least one pharmaceutically acceptable excipient

A “pharmaceutical composition” may comprise a composition described herein (i.e. active ingredient), and one or more pharmaceutically acceptable excipients. The excipient(s) must be acceptable in the sense of being compatible with the other ingredients of the formulation, capable of pharmaceutical formulation, not deleterious to the recipient thereof, and/or do not interfere with the efficacy of the active ingredient. Therefore, pharmaceutical compositions of the invention are suitable for administration to a patient.

As used herein, “pharmaceutically acceptable excipient” may include one or more of buffering agents, water, saline, phosphate buffered saline, dextrose, glycerol, ethanol and the like, as well as combinations thereof. In many cases, it will be preferable to include isotonic agents, for example, polyol, sugars, polyalcohols such as mannitol, sorbitol, or sodium chloride, preservatives; co-solvents; antioxidants including ascorbic acid and methionine; chelating agents such as EDTA; metal complexes (e.g. Zn²⁺-protein complexes); biodegradable polymers; and/or salt-forming counterions such as sodium or potassium.

The precise nature of the excipient or other material may depend on the route of administration, which may be, for example, oral, rectal, nasal, topical (including buccal and sublingual), vaginal, parenteral (including subcutaneous, intramuscular, intravenous, intradermal, intrathecal, and epidural), and intratumorally. It will be appreciated that the preferred excipient may vary with, for example, the condition of the recipient and the disease to be treated.

A mixture of excipients and concentrations of each together form a “pharmaceutical formulation” (or “formulation”). Such compositions are suitably free of visible particulate matter. The formulation may be in liquid form or lyophilized form. A composition in a liquid formulation may be filled into containers and frozen. In certain embodiments, aliquots of the frozen formulation comprising the composition may be lyophilized. Lyophilizate may be reconstituted by the addition of water or other aqueous solution to produce a reconstituted formulation comprising the composition.

In one embodiment, the composition is in a liquid formulation. In some embodiments, the formulation comprises the antibody at about 10 mg/mL to about 125 mg/mL. In a further embodiment, the formulation comprises the antibody at about 20 mg/mL to about 125 mg/mL, such as about 20 mg/mL to about 100 mg/mL, in particular about 20 mg/mL to about 50 mg/mL. In a further embodiment, the formulation comprises the antibody at about 20 mg/mL. In an alternative embodiment, the formulation comprises the antibody at about 50 mg/mL.

In one aspect there is provided a formulation comprising the pharmaceutical composition described herein comprising the antibody at about 20 mg/mL to about 125 mg/mL (such as about 20 mg/mL to about 50 mg/mL) and a buffering agent at a pH of about 5.5 to about 6.5. In one embodiment, the composition is in a liquid formulation.

In some embodiments, the composition is formulated as a sterile liquid. In some embodiments, the composition is free from visible particles. In some embodiments, the composition is formulated in a buffer (e.g. a citrate buffer). In some embodiments, the composition comprises a PD-1 antibody and two or more of the following: citrate buffer, histidine buffer arginine, trehalose, sodium chloride and polysorbate 80.

In certain embodiments, a buffering agent is a citrate buffer. Citrate buffer can be achieved, for example, by the use of a conjugate acid/conjugate base system (sodium citrate/citric acid) or by HCl titration of a sodium citrate solution. In one embodiment, the citrate buffer is at a pH of about 5.0 to about 6.5, such as about 5.5 to about 6.0, in particular about 5.5 or about 6.0.

In an alternative embodiment, the buffering agent is a histidine buffer. In one embodiment, the histidine buffer is at a pH of about 5.5 to about 7.0, about 5.5 to about 6.5, such as about 6.0 to about 6.5, in particular about 6.0 or about 6.5.

In some embodiments, a formulation comprises a surfactant. “Surfactants” are surface active agents that can exert their effect at surfaces of solid-solid, solid-liquid, liquid-liquid, and liquid-air interfaces because of their chemical composition, containing both hydrophilic and hydrophobic groups. Surfactants may reduce the concentration of proteins in dilute solutions at the air-water and/or water-solid interfaces where proteins can be adsorbed and potentially aggregated. Surfactants can bind to hydrophobic interfaces in protein formulations. Some parentally acceptable non-ionic surfactants comprise either polysorbate or polyether groups. Polysorbate 20 and 80, in particular polysorbate 80 (PS80), are suitable surfactant stabilizers in formulations of the invention. In one embodiment, the formulation additionally comprises PS80. In some embodiments, a formulation comprises PS80 or PS20 at about 0.01% to about 0.1%, such as about 0.01% to about 0.05% or about 0.01 to about 0.03% w/v. In some embodiments, the formulation comprises PS80 or PS20 at about 0.02% w/v. In a preferred embodiment, a formulation comprises PS80 at about 0.02% w/v.

In some embodiments, the formulation additionally comprises sodium chloride at a concentration to adjust the osmolality of the formulation to about 290-325 mOsm/kg, such as about 290 mOsm/kg. In one embodiment, the formulation comprises sodium chloride at about 20 mM to about 40 mM, such as about 25 mM to about 35 mM. In a further embodiment, the formulation comprises about 31 mM sodium chloride.

The formulation may comprise may also comprise a solubilizer, such as arginine, for example L-Arginine-HCl. In some embodiments, the formulation comprises arginine in the range from about from about 80 mM to about 120 mM, such as about 90 mM to about 110 mM, in particular about 95 mM to about 105 mM. In some embodiments, the formulation comprises about 100 mM arginine.

In some embodiments, the formulation comprises a polyol. In some embodiments, the polyol is a sugar, and preferably a non-reducing sugar. In some embodiments, the non-reducing sugar is trehalose. In some embodiments, the formulation comprises trehalose in the range from about from about 2% to about 10% w/v. In some embodiments, the formulation comprises about 5% w/v trehalose.

In some embodiments, the formulation comprises arginine and/or trehalose, such as arginine at about 80 mM to about 120 mM (in particular 100 mM) or trehalose at about 2% to about 10% w/v (in particular 5% w/v).

In one aspect, there is provided a formulation comprising the pharmaceutical composition, comprising the antibody at about 20 mg/mL to about 125 mg/mL, citrate buffer or histidine buffer at about 10 mM to about 40 mM, arginine at about 80 mM to about 120 mM or trehalose at about 2% to about 10% w/v, sodium chloride at about 20 mM to about 40 mM, and polysorbate 80 at about 0.01% to about 0.03% w/v, at a pH of about 5.5 to about 6.5.

In one aspect, there is provided a formulation comprising about 20-125 mg/mL of the antibody, about 25 mM citrate buffer, about 100 mM arginine (e.g. L-Arginine-HCl), about 31 mM sodium chloride, and about 0.02% (w/v) polysorbate 80, at about pH 6.

In one aspect, there is provided a formulation comprising about 20 mg/mL of the antibody, about 25 mM citrate buffer, about 100 mM arginine, about 31 mM sodium chloride, and about 0.02% (w/v) polysorbate 80, at about pH 6.

In one aspect, there is provided a formulation comprising about 50 mg/mL of the antibody, about 25 mM citrate buffer, about 100 mM arginine, about 31 mM sodium chloride, and about 0.02% w/v polysorbate 80, at about pH 6.

A “stable” formulation is one in which the protein therein essentially retains its physical and/or chemical stability during manufacturing, transport, storage, and administration. Stability can be measured at a selected temperature for a selected time period. For example, for a product stored at a recommended temperature of 2° C. to 8° C., the formulation is stable at room temperature, about 30° C., or at 40° C., for at least 1 month and/or stable at about 2 to 8° C. for at least 1 year and preferably for at least 2 years. For example, the extent of aggregation, acidic variant, and/or basic variant during storage can be used as an indicator of protein stability. Thus, a “stable” formulation may be one wherein, about 10% or less, about 5% or less, for example about 4% or less aggregation variant of the antibody is present in the formulation. A “stable” formulation may be one wherein, about 60% or less, about 50% or less, for example about 35% or less acidic variant of the antibody is present in the formulation. A “stable” formulation may be one wherein, about 35% or less, about 10% or less, for example about 15% or less basic variant of the antibody is present in the formulation.

In certain aspects of the invention, a formulation allows the composition to remain stable to storage at about 2 to 8° C. for at least 18 months, freezing, thawing, and/or mixing. In one embodiment, a “stable” formulation may be one wherein, about 4% or less aggregation variant, about 35% or less acidic variant, and about 15% or less basic variant of the antibody is present in the formulation.

In yet another aspect, the present invention is directed to an article of manufacture, e.g. a kit, comprising a container holding a composition in a formulation described herein. In one aspect there is provided an injection device comprising the formulation. The injection device may comprise a pen injector device or an autoinjector device. In one embodiment, the formulation is contained in a prefilled syringe.

### Methods of Treatment and Compositions for Use

The invention further provides a method of treating any disease or disorder in which the improper expression (e.g. overexpression) or increased activity of a PD-1 protein causes or contributes to the pathological effects of the disease, or a decrease in PD-1 protein levels or activity has a therapeutic benefit in mammals, preferably humans.

In one aspect, there is provided a composition described herein for use in therapy. Such therapy may relate to any disease or disorder in which the improper expression (e.g. overexpression) or increased activity of a PD-1 protein causes or contributes to the pathological effects of the disease, or a decrease in PD-1 protein levels or activity has a therapeutic benefit in mammals, preferably humans.

The compositions may be used in methods of increasing T cell activation or T cell effector function in a subject, which method comprises administering a therapeutically effective dose of an agent that is capable of inhibiting PD-1 signaling. The compositions may be used in methods of inducing an immune response in a subject, which method comprises administering a therapeutically effective dose of an agent that is capable of inhibiting PD-1 signaling. The compositions may be used in methods of enhancing an immune response or increasing the activity of an immune cell in a subject, which method comprises administering a therapeutically effective dose of an agent that is capable of inhibiting PD-1 signaling.

In one aspect, there is provided a composition for use in the treatment of cancer. Alternatively, there is provided a composition for use in the treatment of an infectious disease.

In one aspect, there is provided use of a composition described herein in the manufacture of a medicament for use in the treatment of cancer. Alternatively, there is provided a composition described herein in the manufacture of a medicament for use in the treatment of an infectious disease.

By the term “treating” and grammatical variations thereof as used herein, is meant therapeutic therapy. In reference to a particular condition, treating means: (1) to ameliorate the condition of one or more of the biological manifestations of the condition, (2) to interfere with a) one or more points in the biological cascade that leads to or is responsible for the condition or b) one or more of the biological manifestations of the condition, (3) to alleviate one or more of the symptoms, effects or side effects associated with the condition or treatment thereof, (4) to slow the progression of the condition or one or more of the biological manifestations of the condition or (5) to prevent the onset of one or more of the biological manifestations of the condition.

Treatment can be therapeutic, prophylactic or preventative. The subject will be one who is in need thereof. Those in need of treatment may include individuals already suffering from a particular medical disease, in addition to those who may develop the disease in the future.

Therefore, prophylactic therapy is also contemplated. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof. Prophylactic therapy is appropriate, for example, when a subject is considered at high risk for developing cancer, such as when a subject has a strong family history of cancer or when a subject has been exposed to a carcinogen.

Thus, the methods, antibodies and compositions described herein can be used for prophylactic treatment or preventative treatment if specified. In this case, the described methods, antibodies and compositions can be used to prevent or delay the onset of one or more aspects or symptoms of a disease. The subject can be asymptomatic. The subject may have a genetic predisposition to the disease. A prophylactically effective amount of the composition is administered to such an individual. A prophylactically effective amount is an amount which prevents or delays the onset of one or more aspects or symptoms of a disease described herein.

The methods, antibodies and compositions need not affect a complete cure, or eradicate every symptom or manifestation of the disease to constitute a viable therapeutic treatment. As is recognized in the art, drugs employed as therapeutic agents in methods of treatment may reduce the severity of a given disease state, but need not abolish every manifestation of the disease to be regarded as useful therapeutic agents. Similarly, a prophylactically administered treatment need not be completely effective in preventing the onset of a disease in order to constitute a viable prophylactic agent. Simply reducing the impact of a disease (for example, by reducing the number or severity of its symptoms, or by increasing the effectiveness of another treatment, or by producing another beneficial effect), or reducing the likelihood that the disease will occur (for example by delaying the onset of the disease) or worsen in a subject, is sufficient.

The terms “individual”, “subject” and “patient” are used herein interchangeably and may be is defined broadly to include any person in need of treatment, for example, a person in need of cancer treatment. The subject is typically a human. The subject may also be a mammal, such as a mouse, rat, or primate (e.g. a marmoset or monkey). The subject can be a non-human animal. The antibodies, compositions and methods of the disclosure also have veterinary use. The subject to be treated may be a farm animal, for example, a cow or bull, sheep, pig, ox, goat or horse, or may be a domestic animal such as a dog or cat. The animal may be any age, or a mature adult animal.

The invention also provides a method of treating a cancer, an infectious disease or an autoimmune disease in a mammal.

### Cancer Treatment

The present disclosure provides methods of reducing tumors or inhibiting the growth of tumor cells in a subject, which method comprises administering a therapeutically effective dose of an agent that is capable of inhibiting PD-1 signaling. The method may comprise administering the aforementioned composition to a mammal having a cancer or an infectious disease, whereupon the cancer or infectious disease is treated in the mammal. As discussed herein, PD-1 is abnormally expressed in a variety of cancers and PD-L1 expression in some cancers (e.g. renal cell carcinoma) patients correlates with tumor aggressiveness. The method can be used to treat any type of cancer known in the art, such as, for example, adenocarcinoma, adenocarcinoma of the lung, acute myeloid leukemia (“AML”), acute lymphoblastic leukemia (“ALL”), adrenocortical carcinoma, anal cancer, appendiceal cancer, B-cell derived leukemia, B-cell derived lymphoma, bladder cancer, brain cancer, breast cancer (e.g. triple negative breast cancer (TNBC)), cancer of the fallopian tube(s), cancer of the testes, cerebral cancer, cervical cancer, choriocarcinoma, chronic myelogenous leukemia, a CNS tumor, colon adenocarcinoma, colon cancer, colorectal cancer, diffuse intrinsic pontine glioma (DIPG), diffuse large B cell lymphoma (“DLBCL”), embryonal rhabdomyosarcoma (ERMS), endometrial cancer, epithelial cancer, esophageal cancer, Ewing's sarcoma, follicular lymphoma (“FL”), gall bladder cancer, gastric cancer, gastrointestinal cancer, glioma, head and neck cancer, a hematological cancer, hepatocellular cancer, Hodgkin's lymphoma/primary mediastinal B-cell lymphoma, kidney cancer, kidney clear cell cancer, laryngeal cancer, leukemia, liver cancer, lung cancer, lymphoma, melanoma, Merkel cell carcinoma, mesothelioma, monocytic leukemia, multiple myeloma, myeloma, a neuroblasts—derived CNS tumor, non-Hodgkin's lymphoma (NHL), non-small cell lung cancer (NSCLC), oral cancer, osteosarcoma, ovarian cancer, ovarian carcinoma, pancreatic cancer, peritoneal cancer, primary peritoneal cancer, prostate cancer, relapsed or refractory classic Hodgkin's Lymphoma (cHL), renal cell carcinoma, rectal cancer, salivary gland cancer (e.g. a salivary gland tumor), sarcoma, skin cancer, small cell lung cancer, small intestine cancer, squamous cell carcinoma of the anogenital region (e.g. squamous cell carcinoma of the anus, penis, cervix, vagina, or vulva), squamous cell carcinoma of the esophagus, squamous cell carcinoma of the head and neck (SCHNC), squamous cell carcinoma of the lung, stomach cancer, T-cell derived leukemia, T-cell derived lymphoma, thymic cancer, a thymoma, thyroid cancer, uveal melanoma, urothelial cell carcinoma, uterine cancer, uterine endometrial cancer, uterine sarcoma, vaginal cancer, vulvar cancer, or Wilms tumor.

In some embodiments, a cancer to be treated with the compositions described herein is characterized by microsatellite instability or lack thereof. Microsatellite instability (“MSI”) is or comprises a change that in the DNA of certain cells (such as tumor cells) in which the number of repeats of microsatellites (short, repeated sequences of DNA) is different than the number of repeats that was contained in the DNA from which it was inherited. Microsatellite instability arises from a failure to repair replication-associated errors due to a defective DNA mismatch repair (MMR) system. This failure allows persistence of mismatch mutations all over the genome, but especially in regions of repetitive DNA known as microsatellites, leading to increased mutational load. It has been demonstrated that at least some tumors characterized by MSI-H have improved responses to certain anti-PD-1 agents (Le et al., (2015) N. Engl. J. Med. 372(26):2509-2520; Westdorp et al., (2016) Cancer Immunol. Immunother. 65(10): 1249-1259).

In some embodiments, a cancer has a microsatellite instability status of high microsatellite instability (e.g. MSI-H status). In some embodiments, a cancer has a microsatellite instability status of low microsatellite instability (e.g. MSI-L status). In some embodiments, a cancer has a microsatellite instability status of microsatellite stable (e.g. MSS status). In some embodiments microsatellite instability status is assessed by a next generation sequencing (NGS)-based assay, an immunohistochemistry (IHC)-based assay, and/or a PCR-based assay. In some embodiments, microsatellite instability is detected by NGS. In some embodiments, microsatellite instability is detected by IHC. In some embodiments, microsatellite instability is detected by PCR.

In embodiments, the cancer is associated with a high tumor mutation burden (TMB). In some embodiments, the cancer is associated with high TMB and MSI-H. In some embodiments, the cancer is associated with high TMB and MSI-L or MSS. In some embodiments, the cancer is endometrial cancer associated with high TMB. In some related embodiments, the endometrial cancer is associated with high TMB and MSI-H. In some related embodiments, the endometrial cancer is associated with high TMB and MSI-L or MSS.

In some embodiments, a cancer is a mismatch repair deficient (dMMR) cancer. Microsatellite instability may arise from a failure to repair replication-associated errors due to a defective DNA mismatch repair (MMR) system. This failure allows persistence of mismatch mutations all over the genome, but especially in regions of repetitive DNA known as microsatellites, leading to increased mutational load that may improve responses to certain anti-PD-1 agents.

In some embodiments, a cancer is a hypermutated cancer. In some embodiments, a cancer harbors a mutation in polymerase epsilon (POLE). In some embodiments, a cancer harbors a mutation in polymerase delta (POLD).

In some embodiments, a cancer is endometrial cancer (e.g. MSI-H or MSS/MSI-L endometrial cancer). In some embodiments, a cancer is a MSI-H cancer comprising a mutation in POLE or POLD (e.g. a MSI-H non-endometrial cancer comprising a mutation in POLE or POLD).

In one aspect, there is provided a method of treating cancer comprising administering to a subject in need thereof a therapeutically effective amount of a composition (including a pharmaceutical composition or formulation) described herein.

As used herein, the terms “cancer,” and “tumor” are used interchangeably and, in either the singular or plural form, refer to cells that have undergone a transformation, such as malignant transformation, that makes them pathological to the host organism. Primary cancer cells can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g. by procedures such as computed tomography (CT) scan, magnetic resonance imaging (MRI), X-ray, ultrasound or palpation on physical examination, and/or which is detectable because of the expression of one or more cancer-specific antigens in a sample obtainable from a patient.

In embodiments, a cancer that is a head and neck cancer, a lung cancer (e.g. a non-small cell lung cancer (NSCLC)), a renal cancer, a bladder cancer, a melanoma, Merkel cell carcinoma (see, e.g. Bhatia et al., Curr. Oncol. Rep., 13(6): 488-497 (2011), a cervical cancer, a vaginal cancer, a vulvar cancer, a uterine cancer, a endometrial cancer, an ovarian cancer, a fallopian tube cancer, a breast cancer, a prostate cancer, a salivary gland tumor, a thymoma, a adrenocortical carcinoma, a esophageal cancer, a gastric cancer, a colorectal cancer, an appendiceal cancer, a urothelial cell carcinoma, or a squamous cell carcinoma (e.g. of the lung; of the anogenital region including anus, penis, cervix, vagina, or vulva; or of the esophagus).

In some embodiments, the cancer is a hematological cancer. In some embodiments, the hematological cancer is selected from: Diffuse large B cell lymphoma (“DLBCL”), Hodgkin's lymphoma (“HL”), Non-Hodgkin's lymphoma (“NHL”), Follicular lymphoma (“FL”), acute myeloid leukemia (“AML”), acute lymphoblastic leukemia (“ALL”), or Multiple myeloma (“MM”). In embodiments, a cancer is a blood-borne cancer such as acute lymphoblastic leukemia (“ALL”), acute lymphoblastic B-cell leukemia, acute lymphoblastic T-cell leukemia, acute myeloblastic leukemia (“AML”), acute promyelocytic leukemia (“APL”), acute monoblastic leukemia, acute erythroleukemic leukemia, acute megakaryoblastic leukemia, acute myelomonocytic leukemia, acute non-lymphocytic leukemia, acute undifferentiated leukemia, chronic myelocytic leukemia (“CML”), chronic lymphocytic leukemia (“CLL”), hairy cell leukemia and multiple myeloma; acute and chronic leukaemias such as lymphoblastic, myelogenous, lymphocytic, and myelocytic leukaemias.

In some embodiments the cancer is a lymphoma such as Hodgkin's disease, non-Hodgkin's Lymphoma, Multiple myeloma, Waldenstrom's macroglobulinemia, Heavy chain disease and Polycythemia vera.

In some embodiments, the cancer is a squamous cell carcinoma. In some embodiments, the cancer is squamous cell carcinoma of the lung. In some embodiments, the cancer is squamous cell carcinoma of the esophagus. In some embodiments, the cancer is head and neck squamous cell carcinoma (HNSCC). In some embodiments, the cancer is squamous cell carcinoma of the anogenital region (e.g. of the anus, penis, cervix, vagina, or vulva).

In some embodiments, the cancer is bladder cancer, breast cancer (e.g. triple negative breast cancer (TNBC)), cancer of the fallopian tube(s), cholagiocarcinoma, colon adenocarcinoma, endometrial cancer, esophageal cancer, Ewing's sarcoma, gastric cancer, kidney clear cell cancer, lung cancer (e.g. lung adenocarcinoma or lung squamous cell cancer), mesothelioma, ovarian cancer, pancreatic cancer, peritoneal cancer, prostate cancer, uterine endometrial cancer, or uveal melanoma. In some embodiments, the cancer is ovarian cancer, cancer of the fallopian tube(s), or peritoneal cancer. In some embodiments, the cancer is breast cancer (e.g. TNBC). In some embodiments, the cancer is lung cancer (e.g. non-small cell lung cancer). In some embodiments, the cancer is prostate cancer.

In some embodiments, the cancer is a CNS or brain cancer such as neuroblastoma (NB), glioma, diffuse intrinsic pontine glioma (DIPG), pilocytic astrocytoma, astrocytoma, anaplastic astrocytoma, glioblastoma multiforme, medulloblastoma, craniopharyngioma, ependymoma, pinealoma, hemangioblastoma, acoustic neuroma, oligodendroglioma, meningioma, vestibular schwannoma, adenoma, metastatic brain tumor, meningioma, spinal tumor, or medulloblastoma. In embodiments, a cancer is a CNS tumor.

In some embodiments, the cancer is a solid tumor. In embodiments, a cancer is a solid tumor such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, osteosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms tumor, cervical cancer, uterine cancer, testicular cancer, non-small cell lung cancer (NSCLC), small cell lung carcinoma, bladder carcinoma, lung cancer, epithelial carcinoma, skin cancer, melanoma, neuroblastoma (NB), or retinoblastoma. In some embodiments, the tumor is an advanced stage solid tumor. In some embodiments, the tumor is a metastatic solid tumor.

In some embodiments, the cancer is a gynecologic cancer (i.e. a cancer of the female reproductive system such as ovarian cancer, fallopian tube cancer, cervical cancer, vaginal cancer, vulvar cancer, uterine cancer, or primary peritoneal cancer, or breast cancer). In some embodiments, cancers of the female reproductive system include, but are not limited to, ovarian cancer, cancer of the fallopian tube(s), peritoneal cancer, and breast cancer.

In some embodiments, the cancer is ovarian cancer (e.g. serous or clear cell ovarian cancer). In some embodiments, the cancer is fallopian tube cancer (e.g. serous or clear cell fallopian tube cancer). In some embodiments, the cancer is primary peritoneal cancer (e.g. serous or clear cell primary peritoneal cancer).

In some embodiments, the ovarian cancer is an epithelial carcinoma. Epithelial carcinomas make up 85% to 90% of ovarian cancers. While historically considered to start on the surface of the ovary, new evidence suggests at least some ovarian cancer begins in special cells in a part of the fallopian tube. The fallopian tubes are small ducts that link a woman's ovaries to her uterus that are a part of a woman's reproductive system. In a normal female reproductive system, there are two fallopian tubes, one located on each side of the uterus. Cancer cells that begin in the fallopian tube may go to the surface of the ovary early on. The term “ovarian cancer” is often used to describe epithelial cancers that begin in the ovary, in the fallopian tube, and from the lining of the abdominal cavity, call the peritoneum. In some embodiments, the cancer is or comprises a germ cell tumor. Germ cell tumors are a type of ovarian cancer develops in the egg-producing cells of the ovaries. In some embodiments, the cancer is or comprises a stromal tumor. Stromal tumors develop in the connective tissue cells that hold the ovaries together, which sometimes is the tissue that makes female hormones called estrogen. In some embodiments, the cancer is or comprises a granulosa cell tumor. Granulosa cell tumors may secrete estrogen resulting in unusual vaginal bleeding at the time of diagnosis. In some embodiments, the gynecologic cancer is associated with homologous recombination repair deficiency/homologous repair deficiency (“HRD”) and/or BRCA1/2 mutation(s). In some embodiments, the gynecologic cancer is platinum-sensitive. In some embodiments, the gynecologic cancer has responded to a platinum-based therapy. In some embodiments, the gynecologic cancer has developed resistance to a platinum-based therapy. In some embodiments, the gynecologic cancer has at one time shown a partial or complete response to platinum-based therapy (e.g. a partial or complete response to the last platinum-based therapy or to the penultimate platinum-based therapy). In some embodiments, the gynecologic cancer is now resistant to platinum-based therapy.

In some embodiments, the cancer is a breast cancer. Usually breast cancer either begins in the cells of the milk producing glands, known as the lobules, or in the ducts. Less commonly breast cancer can begin in the stromal tissues. These include the fatty and fibrous connective tissues of the breast. Over time the breast cancer cells can invade nearby tissues such the underarm lymph nodes or the lungs in a process known as metastasis. The stage of a breast cancer, the size of the tumor and its rate of growth are all factors which determine the type of treatment that is offered. Treatment options include surgery to remove the tumor, drug treatment which includes chemotherapy and hormonal therapy, radiation therapy and immunotherapy. The prognosis and survival rate varies widely; the five year relative survival rates vary from 98% to 23% depending on the type of breast cancer that occurs. Breast cancer is the second most common cancer in the world with approximately 1.7 million new cases in 2012 and the fifth most common cause of death from cancer, with approximately 521,000 deaths. Of these cases, approximately 15% are triple-negative, which do not express the estrogen receptor, progesterone receptor (PR) or HER2. In some embodiments, triple negative breast cancer (TNBC) is characterized as breast cancer cells that are estrogen receptor expression negative (<1% of cells), progesterone receptor expression negative (<1% of cells), and HER2-negative.

In some embodiments, the cancer is ER-positive breast cancer, ER-negative breast cancer, PR-positive breast cancer, PR-negative breast cancer, HER2-positive breast cancer, HER2-negative breast cancer, BRCA1/2-positive breast cancer, BRCA1/2-negative cancer, or TNBC. In embodiments, a cancer is TNBC.

In some embodiments, the breast cancer is a metastatic breast cancer. In some embodiments, the breast cancer is an advanced breast cancer. In some embodiments, the cancer is a stage II, stage III or stage IV breast cancer. In some embodiments, the cancer is a stage IV breast cancer.

In some embodiments, the cancer is endometrial cancer (“EC”). In some embodiments, the endometrial cancer is metastatic endometrial cancer.

Endometrial carcinoma is the most common cancer of the female genital tract. The annual number of new cases of endometrial cancer (EC) is estimated at about 325,000 worldwide. Further, EC is the most commonly occurring cancer in postmenopausal women. About 53% of endometrial cancer cases occur in developed countries. In 2015, approximately 55,000 cases of EC were diagnosed in the U.S. and no targeted therapies are currently approved for use in EC. There is a need for agents and regimens that improve survival for advanced and recurrent EC in first line (1 L) and second line (2L) therapy settings. The most common histologic form is endometrioid adenocarcinoma, representing about 75-80% of diagnosed cases. Other histologic forms include uterine papillary serous (less than 10%), clear cell 4%, mucinous 1%, squamous less than 1% and mixed about 10%.

From the pathogenetic point of view, EC falls into two different types, so-called types I and II. Type I tumors are low-grade and estrogen-related endometrioid carcinomas (EEC) while type are non-endometrioid (NEEC) (mainly serous and clear cell) carcinomas. The World Health Organization has recently updated the pathologic classification of EC, recognizing nine different subtypes of EC, but EEC and serous carcinoma (SC) account for the vast majority of cases. EECs are estrogen-related carcinomas, which occur in perimenopausal patients, and are preceded by precursor lesions (endometrial hyperplasia/endometrioid intraepithelial neoplasia). Microscopically, low-grade EEC (EEC 1-2) contains tubular glands, somewhat resembling the proliferative endometrium, with architectural complexity with fusion of the glands and cribriform pattern. High-grade EEC shows solid pattern of growth. In contrast, SC occurs in postmenopausal patients in absence of hyper-estrogenism. At the microscope, SC shows thick, fibrotic or edematous papillae with prominent stratification of tumor cells, cellular budding, and anaplastic cells with large, eosinophilic cytoplasms. The vast majority of EEC are low grade tumors (grades 1 and 2), and are associated with good prognosis when they are restricted to the uterus. Grade 3 EEC (EEC3) is an aggressive tumor, with increased frequency of lymph node metastasis. SCs are very aggressive, unrelated to estrogen stimulation, mainly occurring in older women. EEC3 and SC are considered high-grade tumors. SC and EEC3 have been compared using the surveillance, epidemiology and End Results (SEER) program data from 1988 to 2001. They represented 10% and 15% of EC respectively, but accounted for 39% and 27% of cancer death respectively.

In some embodiments, the cancer is a lung cancer. In some embodiments, the lung cancer is a squamous cell carcinoma of the lung. In some embodiments, the lung cancer is small cell lung cancer (SCLC). In some embodiments, the lung cancer is non-small cell lung cancer (NSCLC) such as squamous NSCLC. In some embodiments, the lung cancer is an ALK-translocated lung cancer (e.g. ALK-translocated NSCLC). In some embodiments, the lung cancer is an EGFR-mutant lung cancer (e.g. EGFR-mutant NSCLC).

In some embodiments, the cancer is a metastatic cancer.

In some embodiments, the cancer is a recurrent cancer (e.g. a recurrent gynecological cancer such as recurrent epithelial ovarian cancer, recurrent fallopian tube cancer, recurrent primary peritoneal cancer, or recurrent endometrial cancer).

The subject in need of cancer treatment may include patients from a variety of stages including newly diagnosed, relapsed, refractory, progressive disease, remission, and others. The subject in need of cancer treatment may also include patients who have undergone stem cell transplant or who are considered transplant ineligible.

### Treatment of Infections

The method can be used to treat any type of infectious disease (i.e. a disease or disorder caused by a bacterium, a virus, a fungus, or a parasite). Examples of infectious diseases that can be treated by the method include, but are not limited to, diseases caused by a human immunodeficiency virus (HIV), a respiratory syncytial virus (RSV), an influenza virus, a dengue virus, a hepatitis B virus (HBV, or a hepatitis C virus (HCV)).

Administration of a composition may induce an immune response against a cancer or infectious disease in a mammal. An “immune response” can entail, for example, antibody production and/or the activation of immune effector cells (e.g. T-cells).

### Treatment of Autoimmune Disease

The method can be used to treat any type of autoimmune disease (i.e. as disease or disorder caused by immune system over-activity in which the body attacks and damages its own tissues), such as those described in, for example, MacKay I. R. and Rose N. R., eds., The Autoimmune Diseases, Fifth Edition, Academic Press, Waltham, Mass. (2014). Examples of autoimmune diseases that can be treated by the compositions include, but are not limited to, multiple sclerosis, type 1 diabetes mellitus, rheumatoid arthritis, scleroderma, Crohn's disease, psoriasis, systemic lupus erythematosus (SLE), and ulcerative colitis. When the method treats an autoimmune disease, an anti-TIM-3 antibody agent can be used in combination with an anti-inflammatory agent including, for example, corticosteroids (e.g. prednisone and fluticasone) and non-steroidal anti-inflammatory drugs (NSAIDs) (e.g. aspirin, ibuprofen, and naproxen).

### Routes of Administration

Subjects may have had at least one prior cancer therapy before being treated with the compositions of the present invention. In one embodiment, the subject has been treated with at least 1, at least 2, at least 3, at least 4, at least 5, at least 6, or at least 7 prior cancer therapies before being treated with the compositions of the present invention. In another embodiment, the subject has newly diagnosed cancer and has had 0 prior therapies before being treated with the compositions of the present invention.

The compositions of the invention may be administered by any appropriate route. For some compositions, suitable routes include oral, rectal, nasal, topical (including buccal and sublingual), vaginal, parenteral (including subcutaneous, intramuscular, intravenous, intradermal, intrathecal, and epidural), and intratumorally. It will be appreciated that the preferred route may vary with, for example, the condition of the recipient and the cancer to be treated.

In some embodiments, the composition is administered intravenously (e.g. by intravenous (IV) infusion). In a further embodiment, the composition is administered through a 30 minute IV infusion.

In some embodiments, the composition is administered by injection. Therefore, in one aspect there is provided an injection device comprising the composition, pharmaceutical composition or formulation of the invention. The injection device may comprise a pen injector device or an autoinjector device.

In one embodiment, the composition is contained in a prefilled syringe.

The desired dosage can be delivered by a single bolus administration of the composition, by multiple bolus administrations of the composition, or by continuous infusion administration of the composition.

In certain embodiments, a composition of the invention is administered as a pharmaceutical composition.

The term “administering” as used herein is meant to refer to the delivery of the compositions described herein to achieve a therapeutic objective. The compositions may be administered at an administration interval for a period sufficient to achieve clinical benefit.

The composition may be administered to the subject in such a way as to target therapy to a particular site.

In certain embodiments, a composition can be co-administered to a subject with one or more additional therapeutic agents. In another embodiment, a composition can be co-administered to a subject with one or more additional cancer therapeutics. The additional cancer therapeutic agent may include, but is not limited to, other immunomodulatory drugs, therapeutic antibodies, CAR-T therapeutics, BiTEs, HDAC inhibitors, proteasome inhibitors, anti-inflammatory compounds, and immunomodulatory imide drugs (IMiD).

“Co-administered” means the administration of two or more different pharmaceutical compositions or treatments (e.g. radiation treatment) that are administered to a subject by combination in the same pharmaceutical composition or separate pharmaceutical compositions. Thus co-administration involves administration at the same time of a single pharmaceutical composition comprising two or more pharmaceutical agents or administration of two or more different compositions to the same subject at the same or different times.

For example, an anti-PD-1 antibody can be administered in combination with other agents for the treatment or prevention of the diseases disclosed herein, such as agents that are cytotoxic to cancer cells, modulate the immunogenicity of cancer cells, or promote immune responses to cancer cells. In this respect, for example, the composition can be used in combination with at least one other anticancer agent including, for example, any chemotherapeutic agent known in the art, ionization radiation, small molecule anticancer agents, cancer vaccines, biological therapies (e.g. other monoclonal antibodies, cancer-killing viruses, gene therapy, and adoptive T-cell transfer), and/or surgery. In some embodiments, a subject (e.g. a mammal, e.g. a human) for treatment with an anti-PD-1 antibody has been treated or will be treated with chemotherapy (e.g. platinum-based chemotherapy). In some embodiments, a chemotherapeutic agent is actinomycin, all-trans retinoic acid, azacitidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil, gemcitabine, hydroxyurea, idarubicin, imatinib, irinotecan, mechlorethamine, mercaptopurine, methotrexate, mitoxantrone, oxaliplatin, paclitaxel, pemetrexed, teniposide, tioguanine, topotecan, valrubicin, vemurafenib, vinblastine, vincristine, vindesine, or vinorelbine. In some such embodiments, a chemotherapeutic agent is a platinum-based chemotherapeutic agent, such as cisplatin, carboplatin, oxaliplatin, nedaplatin, triplatin tetranitrate, phenanthriplatin, picoplatin, or satraplatin. In some such embodiments, a chemotherapeutic agent is a folate antimetabolite such as pemetrexed. In some embodiments, a subject (e.g. a mammal, e.g. a human) for treatment with an anti-PD-1 antibody has been treated or will be treated with an anti-angiogenic agent, for example, bevacizumab, itraconazole, carboxyamidotriazole, TNP-470, fumagillin, CM101, IL-12, platelet factor-4, suramin, SU5416, thrombospondin, angiostatic steroids, heparin, cartilage-derived angiogenesis inhibitory factor (e.g. peptide troponin I and chondromodulin I), matrix metalloproteinase inhibitor, angiostatin, endostatin, 2-methoxyestradiol, tecogalan, tetrathiomolybdate, thrombospondin, thalidomide, prolactin, αvβ3 inhibitor, lenalidomide, linomide, ramucirumab, tasquinimod, ranibizumab, sorafenib, sunitinib, pazopanib, everolimus, tissue inhibitors of metalloproteases (TIMP1 and TIMP2), bFGF soluble receptor, transforming growth factor beta, interferon alpha, interferon beta, soluble KDR and FLT-1 receptors, placental proliferin-related protein, pazopanib, sunitinib, sorafenib, axitinib, ponatinib, cabozantinib, regorafenib, vandetanib, lenvatinib, semaxanib, SU6668, vatalanib, tivozanib, cediranib, protamine, heparin, steroids, ascorbic acid ethers, sulphated polysaccharide DS 4152, AGM 12470, neovastat, R04929097, MRK-003, MK-0752, PF03084014, MED10639, curcumin, 3,3′-diindolylmethane (DIM), resveratrol, 3,5-bis(2,4-difluorobenzylidene)-4-piperidone (DiFiD) and epigallocatechin-3-gallate (EGCG), honokiol, Flt2-11, CBO-P11, Je-11, V1, and any combination thereof. In some embodiments, the composition can be used in combination with an anti-inflammatory agent including, for example, corticosteroids (e.g. prednisone and fluticasone) and non-steroidal anti-inflammatory drugs (NSAIDs) (e.g. aspirin, ibuprofen, and naproxen).

In some embodiments, the composition is used to treat an infectious disease. When the inventive method treats an infectious disease, an anti-PD-1 antibody agent can be administered in combination with at least one antibacterial agent or at least one anti-viral agent. In this respect, the anti-bacterial agent can be any suitable antibiotic known in the art. The anti-viral agent can be any vaccine of any suitable type that specifically targets a particular virus (e.g. live-attenuated vaccines, subunit vaccines, recombinant vector vaccines, and small molecule anti-viral therapies (e.g. viral replication inhibitors and nucleoside analogs).

In some embodiments, the composition can be administered in combination with other agents that inhibit immune checkpoint pathways. For example, the composition can be administered in combination with agents that inhibit or antagonize the CTLA-4, TIM-3 or LAG-3 pathways. Combination treatments that simultaneously target two or more of these immune checkpoint pathways have demonstrated improved and potentially synergistic anti-tumor activity. In some embodiments, the composition is administered in combination with an antibody that binds to TIM-3 and/or an antibody that binds to LAG-3. In this respect, the inventive method of treating a cancer or an infectious disease in a mammal can further comprise administering to the mammal a composition comprising (i) an antibody that binds to a TIM-3 protein and/or (ii) an antibody that binds to a LAG-3 protein, optionally in combination with a pharmaceutically acceptable carrier. Exemplary antibody agents specific for LAG-3 and TIM-3 are described in WO2016/126858 and WO2016/161270, respectively, both of which are hereby incorporated by reference. In some embodiments, an anti-TIM-3 antibody agent can be used in combination with an anti-inflammatory agent including, for example, corticosteroids (e.g. prednisone and fluticasone) and non-steroidal anti-inflammatory drugs (NSAIDs) (e.g. aspirin, ibuprofen, and naproxen).

In some embodiments, a subject is receiving or will receive one or more additional therapies in combination with an anti-PD-1 antibody agent. In some embodiments, an additional therapy is a PARP inhibitor. The PARP inhibitor may be, for example, selected from: ABT-767, AZD 2461, BGB-290, BGP 15, CEP 8983, CEP 9722, DR 2313, E7016, E7449, fluzoparib (SHR 3162), IMP 4297, INO1001, JPI 289, JPI 547, monoclonal antibody B3-LysPE40 conjugate, MP 124, niraparib (ZEJULA) (MK-4827), NU 1025, NU 1064, NU 1076, NU1085, olaparib (AZD2281), ON02231, PD 128763, R 503, R 554, rucaparib (RUBRACA) (AG-014699, PF-01367338), SBP 101, SC 101914, simmiparib, talazoparib (BMN-673), veliparib (ABT-888), WW 46, 2-(4-(trifluoromethyl)phenyl)-7,8-dihydro-5H-thiopyrano[4,3-d]pyrimidin-4-ol, and salts or derivatives thereof. In some embodiments, a PARP inhibitor is selected from the group consisting of: niraparib, olaparib, rucaparib, talazoparib, and veliparib. In some embodiments, the PARP inhibitor is niraparib (e.g. niraparib free base, niraparib tosylate, or niraparib tosylate monohydrate, or any combination thereof).

In some embodiments, additional therapies include treatment with a composition that delivers an agent that inhibits TIM-3 or LAG-3 and treatment with a PARP inhibitor such that the subject receives treatment with all three. In some embodiments, additional therapies include treatment with a composition that delivers an agent that inhibits TIM-3, treatment with a composition that delivers an agent that inhibits LAG-3, and treatment with a PARP inhibitor such that the subject receives treatment with all four.

In some embodiments, the method comprises administering the composition in combination with niraparib. Such methods may optionally include administration of an anti-angiogenic agent, such as bevacizumab. In some embodiments, the combination is for administration to patients with an ovarian cancer, a head and neck cancer, a lung cancer (e.g. a non-small cell lung cancer (NSCLC)), a renal cancer, a bladder cancer, a melanoma, Merkel cell carcinoma, a cervical cancer, a vaginal cancer, a vulvar cancer, a uterine cancer, a endometrial cancer, a fallopian tube cancer, a breast cancer, a prostate cancer, a salivary gland tumor, a thymoma, a adrenocortical carcinoma, a esophageal cancer, a gastric cancer, a colorectal cancer, an appendiceal cancer, a urothelial cell carcinoma, or a squamous cell carcinoma (e.g. of the lung; of the anogenital region including anus, penis, cervix, vagina, or vulva; or of the esophagus). In further embodiments, the cancer is selected from ovarian cancer or lung cancer (e.g. NSCLC).

In some embodiments, the method comprises administering the composition in combination with niraparib, in particular to a patient having a recurrent and/or platinum sensitive cancer. In some embodiments, a recurrent and/or platinum sensitive cancer is an ovarian cancer, a head and neck cancer, a lung cancer (e.g. a non-small cell lung cancer (NSCLC)), a renal cancer, a bladder cancer, a melanoma, Merkel cell carcinoma, a cervical cancer, a vaginal cancer, a vulvar cancer, a uterine cancer, a endometrial cancer, a fallopian tube cancer, a breast cancer, a prostate cancer, a salivary gland tumor, a thymoma, a adrenocortical carcinoma, a esophageal cancer, a gastric cancer, a colorectal cancer, an appendiceal cancer, a urothelial cell carcinoma, or a squamous cell carcinoma (e.g. of the lung; of the anogenital region including anus, penis, cervix, vagina, or vulva; or of the esophagus). In some certain embodiments, a recurrent and/or platinum sensitive cancer is an ovarian cancer, an anal cancer, a fallopian tube cancer, or a lung cancer. In some certain embodiments, a recurrent and/or platinum sensitive cancer is an endometrial cancer, triple negative breast cancer, ovarian cancer, non-small cell lung cancer (NSCLC), squamous cell carcinoma of the lung or squamous cell carcinoma of the anogenital region (e.g. squamous cell carcinoma of the anus, penis, cervix, vagina, or vulva). In a further embodiment, the recurrent and/or platinum sensitive cancer is an ovarian cancer. Such methods may optionally include administration of an anti-angiogenic agent, such as bevacizumab.

### Dosage

The compositions described herein may be administered in a therapeutically effective amount.

The term “therapeutically effective amount” or “therapeutically effective dose” of a composition as used herein refers to an amount of an agent (such as an antibody or a pharmaceutical composition) which provides a therapeutic benefit in the treatment or management of one or more symptoms of a condition to be treated.

Therapeutically effective amounts and treatment regimes are generally determined empirically and may be dependent on factors, such as the age, weight, and health status of the patient and disease or disorder to be treated. Such factors are within the purview of the attending physician.

Ranges provided herein, of any type, include all values within a particular range described and values about an endpoint for a particular range.

In some embodiments, a therapeutically effective dose is a flat dose of about 100-2000 mg (e.g. a flat dose about 100 mg; a flat dose about 200 mg; a flat dose about 300 mg; a flat dose about 400 mg; a flat dose about 500 mg; a flat dose about 600 mg; a flat dose about 700 mg; a flat dose about 800 mg; a flat dose about 900 mg; a flat dose about 1000 mg; a flat dose about 1100 mg; a flat dose about 1200 mg; a flat dose about 1300 mg; a flat dose about 1400 mg; a flat dose about 1500 mg; a flat dose about 1600 mg; a flat dose about 1700 mg; a flat dose about 1800 mg; a flat dose about 1900 mg; or a flat dose about 2000 mg). In some embodiments, a therapeutically effective dose is about 1 mg/kg. In some embodiments, a therapeutically effective dose is about 3 mg/kg. In some embodiments, a therapeutically effective dose is about 10 mg/kg. In some embodiments, a therapeutically effective dose is a flat dose about 500 mg. In some embodiments, a therapeutically effective dose is about 800 mg. In some embodiments, a therapeutically effective dose is about 1000 mg.

In one embodiment, the composition is administered once every 2-6 weeks (e.g. 2, 3 or 4 weeks, in particular 3 weeks).

In one embodiment, the composition is administered for once every 3 weeks for 2-6 dosing cycles (e.g. the first 3, 4, or 5 dosing cycles, in particular, the first 4 dosing cycles).

In one embodiment, the composition is administered every three weeks for four doses at 500 mg, then administered every six weeks until disease progression at 1000 mg.

If desired, the effective daily dose of a (therapeutic) composition may be administered as two, three, four, five, six or more doses administered separately at appropriate intervals throughout the day, optionally, in unit dosage forms.

The present disclosure provides methods of treating cancer comprising administering to a patient in need of treatment the composition at a first dose at a first interval for a first period; and administering to the patient the composition at a second dose at a second interval for a second period.

In some embodiments, the first dose and second dose are different. In some embodiments, the first dose is about 500 mg and the second dose is about 1000 mg.

In some embodiments, the first interval and the second interval are different. In some embodiments, the first interval is once every three weeks and the second interval is once every six weeks. In some embodiments, composition is administered at the first dose of 500 mg once every three weeks for the first period of 2-6 dosing cycles (e.g. the first 3, 4, or 5 dosing cycles, in particular, the first 4 dosing cycles), and at the second dose of 1000 mg once every six weeks until therapy is discontinued (e.g. due to disease progression, an adverse event, or as determined by a physician). In some embodiments, the composition is administered at the first dose of 500 mg once every three weeks for the first three dosing cycles, and at the second dose of 1000 mg once every six weeks or more until therapy is discontinued (e.g. due to disease progression, an adverse event, or as determined by a physician). In some embodiments, the composition is administered at the first dose of 500 mg once every three weeks for the first four dosing cycles, and at the second dose of 1000 mg once every six weeks or more until therapy is discontinued (e.g. due to disease progression, an adverse event, or as determined by a physician). In some embodiments, the composition is administered at the first dose of 500 mg once every three weeks for the first five dosing cycles, and at the second dose of 1000 mg once every six weeks or more until therapy is discontinued (e.g. due to disease progression, an adverse event, or as determined by a physician). In some embodiments, the second dose is administered once every six weeks.

In some embodiments, the composition is administered at an administration interval (or treatment cycle) of once a week (Q1W), once every 2 weeks (Q2W), once every 3 weeks (Q3W), once every 4 weeks (Q4W), once every 5 weeks (Q5W), or once every 6 weeks (Q6W). In some embodiments, the composition is administered at an administration interval (or treatment cycle) of once a week (Q1W). In some embodiments, the composition is administered at an administration interval (or treatment cycle) of once every 2 weeks (Q2W). In some embodiments, the composition is administered at an administration interval (or treatment cycle) of once every three weeks (Q3W). In some embodiments, the composition is administered at an administration interval (or treatment cycle) of once every 4 weeks (Q4W). In some embodiments, the composition is administered at an administration interval (or treatment cycle) of once every 5 weeks (Q5W). In some embodiments, the composition is administered at an administration interval (or treatment cycle) of once every 6 weeks (Q6W). In some embodiments, the composition is administered for a period of at least about 2, 3, 4, 5, 6, 7, 8, 9, 10, 11, 12, 13, 14, 15, 16, 17, 18, 19, 20 weeks, or more. In some embodiments, the composition is administered on the first day of a treatment cycle or within 1, 2, or 3 days of the first day of a treatment cycle.

In some embodiments, the composition described herein is administered according to dosing regimens demonstrated to achieve a clinical benefit the patient. In some embodiments, a clinical benefit is stable disease (“SD”), a partial response (“PR”) and/or a complete response (“CR”). In some embodiments, a clinical benefit is stable disease (“SD”). In embodiments, a clinical benefit is a partial response (“PR”). In embodiments, a clinical benefit is a complete response (“CR”). In some embodiments, PR or CR is determined in accordance with Response Evaluation Criteria in Solid Tumors (RECIST). In some embodiments, the composition is administered for a longer period to maintain clinical benefit.

All patent and literature references disclosed herein are expressly and entirely incorporated herein by reference.

### EXAMPLES

Dostarlimab is a humanized monoclonal antibody (mAb) of the IgG4 kappa isotype comprised of two heavy and two light chains with a single N-linked glycosylation site on each heavy chain.

Dostarlimab drug substance (DS) batches manufactured with the commercial process and scale (two batches) were used to fully characterize the population of dostarlimab antibodies. Primary, secondary, and higher order structures, along with physicochemical properties, heterogeneity, biological activity, immunochemical properties and degradation pathways were evaluated. The results are in agreement with previous characterization results for the Clinical Reference Standard material (CRS). A summary of the characterization data is presented below. The characterization methods are described briefly in the section where they are first mentioned.

### 1. Amino Acid Sequence Confirmation by Peptide Map

A specific peptide mapping method for the purpose of protein characterization was used to confirm the amino acid sequence. A dostarlimab sample was denatured with guanidine hydrochloride, reduced with dithiothreitol (DTT), alkylated with iodoacetamide, and digested with endoproteinase Lys-C (Lys-C) or trypsin. Enzymatic digestion with either Lys-C or trypsin was accomplished at 37° C. for 4 hours. The sample digestion was quenched with trifluoroacetic acid prior to the liquid chromatography with tandem mass spectrometry (LC-MS/MS) analysis. The LC-MS/MS analysis system employed reverse-phase ultra-high performance liquid chromatography (UHPLC) with a C18 column, UV detection at 214 nm, and electrospray ionization mass spectrometry (ESI-MS).

The results of the Lys-C peptide map confirmed 98.3% of the predicted full amino acid sequence of the light chain and heavy chain. Several small peptides (LL12: H189-K190, LH9: V210-K212, LH17: C317-K318, LH18: V319-K322) were not identified, which is likely due to early elution prior to MS signal data collection. In order to identify these small peptides, a tryptic digest of dostarlimab was analyzed by LC-MS/MS analysis. Mis-cleaved tryptic peptides (TL16-17: H189-K207, TH13-15: T196-R213, TH24-25: C317-K322) were identified, which completes sequence coverage for those missing peptides in Lys-C peptide map.

The combined Lys-C and tryptic peptide map data confirmed 100% of the predicted amino acid sequence.

### 1.1 Terminal Amino Acid Sequence

The N-terminal and C-terminal amino acid sequences were confirmed using the tryptic peptide map. A dostarlimab sample was denatured, reduced, alkylated, and digested with trypsin prior to LC-MS/MS analysis.

The N-terminal or C-terminal amino acid on the peptides were not detected by MS/MS. This does not impact the confirmation of the terminal amino acid sequence. The combination of the fragmentation pattern in MS/MS data and the measured molecular weight (MW) is sufficient to eliminate other substitution of the terminal amino acid. Thus, the N-terminal and C-terminal sequences of the dostarlimab light chain and heavy chain are consistent with the theoretical sequences.

### 2. Post-Translational Modifications

### 2.1 Glycan Analysis

The single glycosylation site of dostarlimab was determined by comparing peptide LH14-15: T285-K313 in the Lys-C peptide map with its deglycosylated form. Upon deglycosylation with PNGase F, the LH14-15: T285-K313 peptide peak increased in signal, and MS data confirmed the identification of the peak and the glycosylation site as asparagine 293. The peak areas (extracted ion) of the LH14-15: T285-K313 peptide with and without N-glycan were used to calculate a glycosylation occupancy of 99%.

### 2.2 Other Post-Translational Modifications

The extracted ion peak areas from the Lys-C and tryptic peptide maps were used to quantify post-translational modifications (PTMs) to the dostarlimab protein.

### 3. Heterogeneity Analysis

### 3.1 Size Heterogeneity Characterization

Size variants for dostarlimab were assessed by preparatory size-exclusion HPLC (SE-HPLC). Characterization of the SE-HPLC fractions was performed using analytical SE-HPLC, reduced and non-reduced capillary electrophoresis (reduced CE-SDS and non-reduced CE-SDS), capillary isoelectric focusing (cIEF), MSD binding assay, light scattering methods, and analytical ultracentrifugation sedimentation velocity (AUC-SV). The results are summarized below.

### 3.1.1 Size Exclusion Chromatography

By SE-HPLC analysis, dostarlimab elutes predominantly as a monomer, while low levels of high molecular weight species (HMW), which elute earlier than the monomer peak, were also observed (Table 2). Low molecular weight species (LMW) which elute later than monomer peak were less than method quantitation limit.

TABLE 2
SE-HPLC Results for Dostarlimab

| Sample | Monomer % | HMW % | LMW % ^{a} |
|---|---|---|---|
| Dostarlimab | 99.2 | 0.8 | <0.05 |

^{a} Method practical quantitation limit (PQL) = 0.05%.

### 3.1.2 SEC-MALS

Dostarlimab was analyzed by SE-HPLC and multiangle light-scattering (SEC-MALS). The measured MW indicates that the HMW species is comprised of dimerized dostarlimab protein and a monomer with an extra 50 kDa molecule attached to it (likely a monomer with two light chain (LC) subunits, or an LC-LC dimer that is non-covalently bound to the monomer).

3.1.3 cIEF

Capillary isoelectric focusing (cIEF) was used to measure the pI of dostarlimab and separate charge variants. The method quantitates the acidic and basic species as a percentage of the total area peak. A representative electropherogram and charge variant distribution are shown in FIG. 1 and Table 3, respectively.

TABLE 3
cIEF Results for Dostarlimab

| | pI of Main | Acidic | Main | Basic |
|---|---|---|---|---|
| Sample | Peak | Peaks % | Peak % | Peaks %^{a} |
| Dostarlimab | 7.51 | 21.5 | 74.2 | <5.0 |

^{a}Method PQL = 5.0%

### 4. Functional Activity (Potency)

Dostarlimab is an IgG4 mAb that blocks the interaction between programmed cell death protein 1 (PD-1) and its ligands programmed cell death-ligand 1 (PD-L1) and programmed cell death-ligand 2 (PD-L2). PD-1 is a cell surface receptor expressed on T cells that limits T-cell activation through binding to PD-L1 and, to a lesser extent, PD-L2. PD-1 also limits tyrosine kinase signaling from the T cell antigen receptor and co-stimulatory receptors. The PD-1/PD-L1 checkpoint serves as a negative regulator of T cell activity to help control local inflammatory responses and maintain self-tolerance. The bioactivity and binding properties of dostarlimab were assessed using various analytical methods including an MSD potency assay, a cell-based potency assay (bioassay) and Fc Receptor (FcRn) binding, as described below.

### 4.1 MSD Binding

The Meso Scale Discovery (MSD) potency assay employs engineered CHO K1 cells constitutively expressing the PD-1 protein. The activity of dostarlimab is assessed using competitive binding, which measures the dose-dependent ability of dostarlimab to inhibit ligand binding to PD-1 on the CHO K1 cells. The ligand (PD-L1) is used as the ligand in the assay (PD-L1-mFc). The readout is measured using a specific detection antibody mixture that releases an electrochemiluminescence (ECL) signal that can be quantified. Results are reported as percent potency relative to reference material (e.g. control sample).

### 4.2 Cell-Based Bioassay

A cell based potency bioassay was developed using the Promega PD-1/PD-L1 Blockade Bioassay (catalog no. J1250 or J1255) which closely mimics dostarlimab's mechanism of action. Specifically, PD-1 effector cells (Jurkat T cells (Promega #J1155)) expressing human PD-1 and a luciferase reporter gene driven by an nuclear factor of activated T-cell response element (NFAT-RE) are co-cultured with PD-L1 aAPC (artificial antigen presenting cells) (CHO-K1 cells (Promega #J1095)) expressing PD-L1. When the two cell types are co-cultured, the PD-1/PD-L1 interaction inhibits TCR signaling and consequently the NFAT-RE-mediated luminescence. Addition of dostarlimab (an anti-PD-1 antibody that blocks the PD-1/PD-L1 interaction) releases the inhibitory signal, resulting in TCR activation, and NFAT-RE-mediated luminescence. This dostarlimab blocking of the inhibitory signal is dose-dependent, and the resulting luminescence can be quantified using a plate reader. From the signal responses, 4-parameter curves are generated for both the reference material and dostarlimab samples by plotting relative luciferase unit (RLU) on the y-axis vs. log₂ transformed concentrations on the x axis. The median effective concentration (EC50) values are interpolated and used to calculate the potency of the dostarlimab sample relative to that of the reference material.

### 4.2.1 FcRn Binding

Neonatal FcRn binding plays a role in the metabolic fate of IgG molecules in the body. Weak binding of an IgG to FcRn results in a much reduced persistence in serum.

FcRn binding was assessed via BIACORE analysis. Dostarlimab reference material, and HERCEPTIN (IgG1) were each immobilized on sensor chips. Various concentrations of analyte (FcRn) were injected for analysis of the association and dissociation properties. FcRn binding strength for the three samples analyzed was similar, as is expected for IgG1 and IgG4s (See Table 4).

TABLE 4
FcRn Binding Results

| Ligand | Analyte | K_{D} (M) |
|---|---|---|
| Dostarlimab | FcRn | 1.75 × 10⁻⁶ |
| Reference Material | FcRn | 1.69 × 10⁻⁶ |
| HERCEPTIN | FcRn | 1.80 × 10⁻⁶ |

### 5. Degradation Product and Pathway

A forced degradation study was performed on DS made at the commercial scale (batch #1) to evaluate degradation pathways for dostarlimab. Degraded samples were assessed for purity by SE-HPLC, cIEF, reduced CE-SDS and non-reduced CE-SDS. Additionally, the samples were evaluated for function by MSD binding assay, cell-based bioassay and FcRn binding assay. The PTMs were evaluated by reduced Lys-C or Tryptic peptide maps. The study results are summarized in the sections below.

### 5.1 Thermal Degradation Conditions

Dostarlimab samples were incubated at 40° C. or 50° C. for up to 3 weeks. Samples were kept frozen until the time of testing.

The SE-HPLC results showed a slight increase in HMW species after samples were incubated at 40° C. for 3 weeks (Table 5).

Both reduced CE-SDS and non-reduced CE-SDS showed no changes outside analytical variability at the 40° C. condition. These include: LMW, medium molecular weight (MMW), and HMW in reduced CE-SDS; and the “pre-peak” and “post-peak” regions in non-reduced CE-SDS (data not shown).

The cIEF data showed an increase in acidic species (up to 59.1%) compared to unstressed controls for the samples incubated at 50° C. for up to 3 weeks (Table 6).

The peptide map data indicated slightly elevated isomerization at Asp 261/266/276 (the specific site of isomerization was not confirmed), deamidation at Asn 380 and Asn 385, and oxidation at Met 248 and Met 354 at the 40° C. condition (Table 7). Although these minor changes are still within analytical variability, the trend was confirmed by 50° C. data. Thermal degradation at 50° C. for 3 weeks showed the same trend indicated by the 40° C. condition, however, the changes were more pronounced. A faster rate of increasing aggregates and fragments were also observed (data not shown).

The HMW species observed under the 50° C. condition are mainly higher order aggregates, which differs from the dimerization seen in unstressed and 40° C. condition samples. Neither the reduced or the non-reduced CE-SDS showed changes in HMW level but a slight increase in fragments was observed (data not shown).

As described above, for the 50° C. condition, the peptide map data indicated increased isomerization at HC Asp 261/266/276, deamidation at HC Asn 380 and HC Asn 385, and oxidation at HC Met 248, HC Met 354 and HC Met 424. The increase of these PTMs resulted in a corresponding increase of percent acidic species, which is consistent with the forced degradation results from base hydrolysis and oxidative conditions. All other post-translational modification levels were within analytical variability.

The relative potency and FcRn binding were not impacted by the changes observed at 40° C. and 50° C. thermal degradation conditions for up to 3 weeks (Table 8). The minor differences observed were within expected analytical variability.

TABLE 5
SE-HPLC Results for Thermally Degraded Dostarlimab

| Condition | Monomer % | HMW % | LMW % ^{a} |
|---|---|---|---|
| Unstressed Control | 99.1 | 0.9 | ND |
| 40° C., 1 week | 98.9 | 1.0 | <0.05 |
| 40° C., 2 weeks | 98.9 | 1.1 | <0.05 |
| 40° C., 3 weeks | 98.5 | 1.4 | 0.08 |
| 50° C., 1 week | 94.8 | 4.3 | 0.9 |
| 50° C., 2 weeks | 91.1 | 7.7 | 1.3 |
| 50° C., 3 weeks | 87.4 | 11.2 | 1.5 |

Abbreviations: ND = not detected
^{a} Method PQL = 0.05%

TABLE 6
cIEF Results for Thermally Degraded Dostarlimab

| | pI of Main | Acidic | Main | Basic |
|---|---|---|---|---|
| Condition | Peak | Peaks % | Peak % | Peaks % ^{a} |
| Unstressed Control | 7.50 | 21.8 | 73.9 | <5.0 |
| 40° C., 1 week | 7.50 | 25.2 | 69.9 | <5.0 |
| 40° C., 2 weeks | 7.50 | 30.0 | 65.4 | <5.0 |
| 40° C., 3 weeks | 7.50 | 33.8 | 62.1 | <5.0 |
| 50° C., 1 week | 7.50 | 38.7 | 54.0 | 7.3 |
| 50° C., 2 weeks | 7.50 | 50.3 | 41.3 | 8.3 |
| 50° C., 3 weeks | 7.50 | 59.1 | 33.4 | 7.5 |

^{a} Method PQL = 5.0%

TABLE 7
PTM Abundance for Thermally Degraded Dostarlimab

| | | | PTM Abundance (%) ^{a} | | |
|---|---|---|---|---|---|
| | | | Unstressed | 40° C., | 50° C., |
| Peptide | Region | PTM | Control | 3 weeks | 3 weeks |
| LH: D245- | Fc | HC: Met 248 | 3.0 | 5.4 | 7.6 |
| K284 | | Oxidation | | | |
| TH: T252- | Fc | HC: Asp 261/ | 1.0 | 2.8 | 13.1 |
| K284 | | 266/276 | | | |
| | | Isomerization ^{b} | | | |
| TH: V298- | Fc | HC: Asn 311 | 6.0 | 6.5 | 7.4 |
| K313 | | Deamidation | | | |
| LH: G337- | Fc | HC: Met 354 | <1.0 | 1.9 | 4.2 |
| K356 | | Oxidation | | | |
| LH: G367- | Fc | HC: Asn 380 | 8.4 | 10.0 | 14.7 |
| K388 | | Deamidation | | | |
| LH: G367- | Fc | HC: Asn 385 | <1.0 | 2.1 | 5.9 |
| K388 | | Deamidation | | | |
| LH: T389- | Fc | HC: Asp 395/ | <1.0 | <1.0 | 2.2 |
| K410 | | 397/409 | | | |
| | | Isomerization ^{b} | | | |
| LH: S411- | Fc | HC: Met 424 | <1.0 | 1.7 | 3.5 |
| K435 | | Oxidation | | | |
| LH: S411- | Fc | HC: Asn 430 | 1.2 | 1.2 | 2.0 |
| K435 | | Deamidation | | | |
| LH: S436- | C- | HC: Lys 443 | 97.8 | 97.8 | 97.8 |
| K443 | terminal | Clipped | | | |
| LL: V150- | Fab, | LC: Asp 151/ | <1.0 | 1.1 | 2.5 |
| K169 | Non-CDR | 167 | | | |
| | | Isomerization ^{b} | | | |

Abbreviations: LH = Heavy chain peptide from Lys-C digest, LL = Light chain peptide from Lys-C digest, TH = Heavy Chain peptide from tryptic digest, TL = Light chain peptide from tryptic digest
^{a} When PTM levels were <1.0% in all samples, the change was considered insignificant and the results were not listed.
^{b} The modification site was not confirmed by MS/MS

TABLE 8
Relative Potency Results (MSD Binding, Cell-Based Bioassay
and FcRn Binding) for Thermally Degraded Dostarlimab

| | Relative Potency | | FcRn |
|---|---|---|---|
| | MSD | Cell-Based | Binding |
| Condition | Binding | Bioassay | (KD, M) |
| Unstressed Control | 88% | 98% | 2.14 × 10⁻⁶ |
| 40° C., 3 weeks | 88% | 94% | 2.25 × 10⁻⁶ |
| 50° C., 3 weeks | 76% | 86% | 2.29 × 10⁻⁶ |

In summary, the degradation products observed under thermal degradation conditions are HMW species, fragments, isomerization at Asp 261/266/276, deamidation at Asn 380 and Asn 385, and oxidation at Met 248, Met 354 and Met 424.

The combination of attributes below has no negative impact on the relative potency (MSD binding (76%) and bioassay (86%)) and FcRn binding of dostarlimab:
HMW up to 11.2% (from SE-HPLC).
Fragments levels of 4.5-4.6% (from LMW %+MMW % in reduced CE-SDS and pre-peak % in non-reduced CE-SDS)
Isomerization at HC Asp 261/266/276 up to 13.1% (from peptide map)
Deamidation at HC Asn 380 up to 14.7% and HC Asn 385 up to 5.9% (from peptide map)
Oxidation at HC Met 248 up to 7.6%, HC Met 354 up to 4.2% and HC Met 424 up to 3.5% (from peptide map)
Acidic species up to 59.1% (from cIEF)

Up to 7.1% HC N-terminal pyro-glutamate Extrapolating from the data for HMW and potency (bioassay), up to 36% HMW can result in at least 60% potency (bioassay). Up to 26% HMW can result in at least 70% potency (bioassay). This is calculated using a linear slope for 0.9% (control), 1.4% (40° C. at 3 weeks) and 11.2% (50° C. at 3 weeks) HMW samples, which have 98%, 94% and 86% potency (bioassay), respectively.

### 5.2 Acid and Base Hydrolysis

### 5.2.1 Acid Hydrolysis

Dostarlimab samples were titrated to pH 4.0 with HCl and incubated at 25° C. for up to 3 weeks. Upon completion, samples were kept frozen until the time of testing. The initial timepoint sample, T0, was exposed to low pH but with no incubation at the elevated temperature. Once the target condition pH was established in sample, it was immediately frozen to arrest degradation.

The SE-HPLC results showed an increase in HMW species upon initial low pH exposure (Table 9). When HCl was added to sample, there was initial local exposure to extreme low pH which caused dostarlimab to form higher order aggregates. The HMW species increased by approximately 2% during the 3 week incubation and the LMW species remained at trace amounts during the 3-week incubation. The HMW species observed under acid hydrolysis are mainly higher order aggregates, which differs from the dimerization seen in the unstressed sample.

The cIEF data showed no changes in the percentage of acidic peaks between the unstressed and the samples incubated at pH 4.0 for up to 3 weeks (Table 10). The minor differences observed were within expected analytical variability. The decrease in main peak percentage was observed upon initial exposure to low pH with the trend continuing as the incubation time extended. As the main peak percentage decreased, the percentage of basic peaks increased proportionally. Since one of the main components of the basic species is protein aggregates, the increased HMW level demonstrated in SE-HPLC results of these acid treated samples supports this finding. The reduced and non-reduced CE-SDS data showed no changes (not shown). The peptide map data showed no difference in PTM levels related to acid hydrolysis and the observed minor differences were within expected analytical variability (Table 11).

The relative potency and FcRn binding were not impacted by the changes observed at pH 4.0 for up to 3 weeks (Table 12), with differences observed being within expected analytical variability.

TABLE 9
The SE-HPLC Results for Acid Degraded Dostarlimab

| Condition | Monomer % | HMW % | LMW % ^{a} |
|---|---|---|---|
| Unstressed Control | 99.1 | 0.9 | ND |
| T0 (exposed to pH 4.0, | 86.5 | 13.4 | <0.05 |
| no incubation) | | | |
| pH 4.0, 25° C., 3 days | 84.6 | 15.3 | 0.05 |
| pH 4.0, 25° C., 1 week | 84.5 | 15.5 | 0.06 |
| pH 4.0, 25° C., 2 weeks | 84.1 | 15.9 | 0.07 |
| pH 4.0, 25° C., 3 weeks | 84.7 | 15.2 | 0.09 |

Abbreviations: ND = not detected
^{a} Method PQL = 0.05%

TABLE 10
The cIEF Results for Acid Degraded Dostarlimab

| | pI of Main | Acidic | Main | Basic |
|---|---|---|---|---|
| Condition | Peak | Peaks % | Peak % | Peaks % ^{a} |
| Unstressed Control | 7.50 | 21.8 | 73.9 | <5.0 |
| T0 (exposed to pH 4.0, | 7.50 | 24.1 | 62.3 | 13.6 |
| no incubation) | | | | |
| pH 4.0, 25° C., 3 days | 7.50 | 20.1 | 62.6 | 17.4 |
| pH 4.0, 25° C., 1 week | 7.50 | 22.5 | 56.0 | 21.6 |
| pH 4.0, 25° C., 2 weeks | 7.50 | 22.6 | 49.6 | 27.8 |
| pH 4.0, 25° C., 3 weeks | 7.50 | 21.7 | 44.3 | 34.1 |

^{a} Method PQL = 5.0%

TABLE 11
PTM Abundance for Acid Degraded Dostarlimab

| | | | PTM Abundance (%) ^{a} | | |
|---|---|---|---|---|---|
| | | | | | pH 4.0, |
| | | | Unstressed | | 25° C., |
| Peptide | Region | PTM | Control | T0 | 3 w |
| LH: D245- | Fc | HC: Met 248 | 3.0 | 3.8 | 3.1 |
| K284 | | Oxidation | | | |
| TH: T252- | Fc | HC: Asp 261/ | 1.0 | <1.0 | 3.0 |
| K284 | | 266/276 | | | |
| | | Isomerization ^{b} | | | |
| TH: V298- | Fc | HC: Asn 311 | 6.0 | 7.1 | 7.2 |
| K313 | | Deamidation | | | |
| LH: G367- | Fc | HC: Asn 380 | 8.4 | 10.1 | 9.7 |
| K388 | | Deamidation | | | |
| LH: G367- | Fc | HC: Asn 385 | <1.0 | 1.5 | 1.8 |
| K388 | | Deamidation | | | |
| LH: T389- | Fc | HC: Asp 395/ | <1.0 | <1.0 | 2.7 |
| K410 | | 397/409 | | | |
| | | Isomerization ^{b} | | | |
| LH: S411- | Fc | HC: Asn 430 | 1.2 | <1.0 | 1.0 |
| K435 | | Deamidation | | | |
| LH: S436- | C- | HC: Lys 443 | 97.8 | 97.1 | 97.0 |
| K443 | terminal | Clipped | | | |
| LL: S127- | Fab, | LC: Asn 137 | <1.0 | 1.2 | 1.2 |
| K145 | Non-CDR | Deamidation | | | |

Abbreviations: LH = Heavy chain peptide from Lys-C digest, TH = Heavy Chain peptide from tryptic digest, LL = Light chain peptide from Lys-C digest, TL = Light chain peptide from tryptic digest
^{a} PTM levels were not listed when they were <1.0% in all samples
^{b} The modification site was not confirmed by MS/MS

TABLE 12
Relative Potency Results (MSD Binding, Cell-Based Bioassay
and FcRn Binding) for Acid Degraded Dostarlimab

| | Relative Potency | | FcRn |
|---|---|---|---|
| | MSD | Cell-Based | Binding |
| Condition | Binding | Bioassay | (KD, M) |
| Unstressed Control | 88% | 98% | 2.14 × 10⁻⁶ |
| T0 (exposed to pH 4.0, | 99% | 109% | 2.06 × 10⁻⁶ |
| no incubation) | | | |
| pH 4.0, 25° C., 3 weeks | 88% | 125% | 1.98 × 10⁻⁶ |

In summary, the degradation products observed under acid hydrolysis conditions are HMW species (mainly higher order aggregates). In conclusion, HMW up to 15.2% (by SE-HPLC) and basic species up to 34.1%, have no negative impact to relative potency and FcRn binding.

### 5.2.2 Base Hydrolysis

Dostarlimab samples were titrated to pH 9.0 with NaOH and incubated at 40° C. for up to 3 weeks. The 40° C. condition was chosen based on an initial condition screening to ensure a meaningful level of degradation can be observed. Upon completion, samples were kept frozen until the time of testing to arrest degradation. The initial sample, T0, was also exposed to the high pH but with no incubation at the elevated temperature. The T0 sample was immediately frozen once target pH was established in sample.

The SE-HPLC results showed an increase in HMW species upon the initial high pH exposure (Table 13). When NaOH was added to sample, there was initial local exposure to extreme high pH which caused dostarlimab to form higher order aggregates. The HMW species increased approximately 4% during the 3-week incubation. The HMW species observed under base hydrolysis are mainly higher order aggregates, which differ from dimerization seen in unstressed sample. The LMW species was slightly elevated and remained less than 1% during the 3-week incubation. Note that in the SE-HPLC figures, the peak presented as the main peak is the monomer.

The cIEF data showed a decrease in the main peak percentage upon initial high pH exposure, and the trend continued with extended incubation time (Table 14). While the main peak percentage decreased, the percentage of acidic peaks correspondingly increased. Although the percentage of basic peaks appeared to decrease slightly, the changes were still within analytical variability. Both reduced and non-reduced CE-SDS data indicated no changes upon initial high pH exposure (not shown). After the 3-week incubation, increases in fragment and HMW levels were observed. After exposing dostarlimab for 3 weeks to high pH conditions the peptide map analysis indicated elevated isomerization at HC Asp 147, deamidation at HC Asn 380 and HC Asn 385, isomerization at LC Asp 151/167 and oxidation at Met 248 (Table 15). All other PTM levels were within analytical variability. The combination of aggregates, fragments, isomerization, and deamidation caused the charge profile of high pH treated dostarlimab sample shift to the acidic species as the dominant species.

Even though the dostarlimab charge profile shifted, the relative potency and FcRn binding were not impacted by the changes observed at pH 9.0 for up to 1 week (Table 16).

TABLE 13
SE-HPLC Results for Base Degraded Dostarlimab

| Condition | Monomer % | HMW % | LMW % |
|---|---|---|---|
| Unstressed Control | 99.1 | 0.9 | ND |
| T0 (exposed to pH 9.0, | 91.7 | 8.1 | 0.2 |
| no incubation) | | | |
| pH 9.0, 40° C., 3 days | 90.9 | 8.7 | 0.4 |
| pH 9.0, 40° C., 1 week | 90.4 | 9.2 | 0.4 |
| pH 9.0, 40° C., 2 weeks | 89.1 | 10.3 | 0.7 |
| pH 9.0, 40° C., 3 weeks | 88.0 | 11.1 | 0.8 |

Abbreviations: ND = not detected

TABLE 14
cIEF Results for Base Degraded Dostarlimab

| | pI of Main | Acidic | Main | Basic |
|---|---|---|---|---|
| Condition | Peak | Peaks % | Peak % | Peaks % ^{a} |
| Unstressed Control | 7.50 | 21.8 | 73.9 | <5.0 |
| T0 (exposed to pH 9.0, | 7.51 | 27.2 | 70.0 | <5.0 |
| no incubation) | | | | |
| pH 9.0, 40° C., 3 days | 7.50 | 60.3 | 38.0 | <5.0 |
| pH 9.0, 40° C., 1 week | 7.50 | 80.1 | 18.8 | <5.0 |
| pH 9.0, 40° C., 2 weeks | 7.49 | 91.6 | 7.3 | <5.0 |
| pH 9.0, 40° C., 3 weeks | 7.49 | 96.8 | 2.6 | <5.0 |

^{a} Method PQL = 5.0%

TABLE 15
PTM Abundance for Base Degraded Dostarlimab

| | | | PTM Abundance (%) ^{a} | | |
|---|---|---|---|---|---|
| | | | | | pH 9.0, |
| | | | Unstressed | | 40° C., |
| Peptide | Region | PTM | Control | T0 | 1 w |
| TH: G44- | CDR | HC: Asp 62 | <1.0 | <1.0 | 2.3 |
| K65 | | Isomerization | | | |
| TH: N77- | Fab, | HC: Asn 84 | <1.0 | <1.0 | 1.3 |
| R87 | Non-CDR | Deamidation | | | |
| LH: D147- | Fab, | HC: Asp 147 | <1.0 | <1.0 | 20.8 |
| K195 | Non-CDR | Isomerization | | | |
| LH: D245- | Fc | HC: Met 248 | 3.0 | 2.8 | 7.1 |
| K284 | | Oxidation | | | |
| TH: T252- | Fc | HC: Asp 261/ | 1.0 | <1.0 | 1.4 |
| K284 | | 266/276 | | | |
| | | Isomerization ^{b} | | | |
| TH: V298- | Fc | HC: Asn 311 | 6.0 | 6.8 | 7.1 |
| K313 | | Deamidation | | | |
| LH: G337- | Fc | HC: Met 354 | <1.0 | <1.0 | 1.2 |
| K356 | | Oxidation | | | |
| LH: G367- | Fc | HC: Asn 380 | 8.4 | 10.8 | 27.8 |
| K388 | | Deamidation | | | |
| LH: G367- | Fc | HC: Asn 385 | <1.0 | 1.8 | 27.2 |
| K388 | | Deamidation | | | |
| LH: N357- | Fc | HC: Asn 357/ | <1.0 | <1.0 | 1.0 |
| K366 | | Gln 358 | | | |
| | | Deamidation ^{b} | | | |
| LH: S411- | Fc | HC: Met 424 | <1.0 | <1.0 | 2.0 |
| K435 | | Oxidation | | | |
| LH: S411- | Fc | HC: Asn 430 | 1.2 | <1.0 | 1.2 |
| K435 | | Deamidation | | | |
| LH: S436- | C- | HC: Lys 443 | 97.8 | 96.9 | 97.0 |
| K443 | terminal | Clipped | | | |
| LL: S127- | Fab, | LC: Asn 137 | <1.0 | 1.3 | 1.7 |
| K145 | Non-CDR | Deamidation | | | |
| LL: V150- | Fab, | LC: Asp 151/167 | <1.0 | <1.0 | 3.1 |
| K169 | Non-CDR | Isomerization ^{b} | | | |

Abbreviations: LH = Heavy chain peptide from Lys-C digest, LL = Light chain peptide from Lys-C digest, TH = Heavy Chain peptide from tryptic digest, TL = Light chain peptide from tryptic digest
^{a} PTM levels were not listed when they were <1.0% in all samples
^{b} The modification site was not confirmed by MS/MS

TABLE 16
Relative Potency Results (MSD Binding Assay, Cell-Based
Bioassay and FcRn Binding) for Base Degraded Dostarlimab

| | Relative Potency | | FcRn |
|---|---|---|---|
| | MSD | Cell-Based | Binding |
| Condition | Binding | Bioassay | (KD, M) |
| Unstressed Control | 88% | 98% | 2.14 × 10⁻⁶ |
| T0 (exposed to pH 9.0, | 93% | 99% | 2.26 × 10⁻⁶ |
| no incubation) | | | |
| pH 9.0, 40° C., 1 week | 85% | 96% | 2.34 × 10⁻⁶ |

In summary, the degradation products observed under base hydrolysis conditions are HMW species, fragments, isomerization at Asp 147 and Asp 151/167, deamidation at Asn 380 and Asn 385, and oxidation at Met 248. The combination of attributes below has no negative impact to relative potency and FcRn binding:
HMW species up to 9.2% (from SE-HPLC)
Fragments 2.0-4.2% (from LMW %+MMW % reduced CE-SDS and pre-peak % in non-reduced CE-SDS)
Isomerization at HC Asp 147 up to 20.8% (from peptide map)
Deamidation at HC Asn 380 up to 27.8% and HC Asn 385 up to 27.2% (from peptide map)
Isomerization at LC Asp 151/167 up to 3.1% (from peptide map)
Oxidation at HC Met 248 up to 7.1% (from peptide map)
Acidic species up to 96.8% (from cIEF)
Up to 5.2% HC N-terminal pyro-glutamate variant

It is expected that isomerization at HC Asp 147 can go higher than the reported level 20.8% without any impact to relative potency or FcRn binding.

It is expected that deamidation at HC Asn 380 and HC Asn 385 can go higher than the reported levels of 27.8% and 27.2%, without any impact to relative potency or FcRn binding.

### 5.3 Oxidative Conditions

5.3.1 H₂O₂ Treated

Hydrogen peroxide (H₂O₂) was spiked into dostarlimab samples to a final concentration of 0.1% (v:v) H₂O₂. The H₂O₂ spiked samples were incubated at 25° C. for up to 3 weeks. Upon completion, the H₂O₂ treatment was quenched with methionine, and samples were kept frozen until the time of testing. The T0 sample was exposed to the H₂O₂ but without incubation at the elevated temperature. Methionine was added to the T0 sample immediately after H₂O₂ (to quench the reaction and arrest degradation.

The SE-HPLC results of the H₂O₂ treated samples showed no difference in the level of HMW and LMW species (Table 17). Starting from the 1 week incubation time, the cIEF data showed a decrease in the main peak percentage with a corresponding increase in the acidic species percentage (Table 18). The main peak also showed a front shoulder in H₂O₂ treated samples.

No differences were observed in reduced CE-SDS data for all treated samples (not shown). The non-reduced CE-SDS data demonstrated an increased level of fragments starting from 3 days of incubation time (not shown). Note that in the SE-HPLC figures, the peak presented as the main peak is the monomer.

The cIEF and non-reduced CE-SDS data indicated that the fragments caused by H₂O₂ migrated as acidic species in cIEF. The peptide map data showed slightly elevated oxidation levels in the Fc region of the HC at Met 248, Met 354 and Met 424 upon exposure to H₂O₂. The oxidation level at all 3 Met sites reached almost 100% after 2 weeks of incubation (Table 19). The oxidation in the CDR of the HC at Met 34 and Met 103 started to show increases after 1 week of incubation. All other PTM levels were within analytical variability.

The cIEF of the H₂O₂ treated samples showed a change in the shape of the main peak and the charge profile shifted to more acidic species. This demonstrated that oxidized species are located in acidic peak region and cIEF is capable of monitoring for excessive oxidation in dostarlimab.

The relative potency of dostarlimab by the MSD binding assay was not impacted by the changes observed in H₂O₂ treated samples; the differences observed were within expected analytical variability (Table 20). However, the cell-based bioassay demonstrated a drop in the relative potency below 50% after 2 weeks. This drop in potency is related to oxidation in the CDR since the dostarlimab binding region (ligand, PD-1) is located in the CDR. Furthermore, since dostarlimab's mechanism of action does not involve effector function, which has been demonstrated to be low, it is unlikely oxidation in Fc region affects potency. Therefore, changes in the CDR would most impact binding and therefore, potency

The FcRn binding analysis was not impacted upon immediate H₂O₂ exposure, but was reduced as the level of oxidation increased with incubation time (Table 20). After 2 weeks, incubation, oxidation was observed in the CDR region (HC Met 34 at ˜29% and HC Met 103 at ˜86%) and Fc region (close to 100%). Since FcRn binding occurs via the Fc region, it can be deduced that the oxidation in the Fc region contributed to the reduced FcRn binding observed.

TABLE 17
SE-HPLC Results for H₂O₂ Treated Dostarlimab

| Condition | Monomer % | HMW % | LMW % ^{a} |
|---|---|---|---|
| Unstressed Control | 99.1 | 0.9 | ND |
| T0 (exposed to H₂O₂, | 99.1 | 0.8 | <0.05 |
| no incubation) | | | |
| H₂O₂, 25° C., 1 day | 99.0 | 0.9 | <0.05 |
| H₂O₂, 25° C., 3 days | 99.1 | 0.9 | 0.05 |
| H₂O₂, 25° C., 1 week | 99.1 | 0.9 | <0.05 |
| H₂O₂, 25° C., 2 weeks | 98.9 | 1.0 | 0.07 |
| H₂O₂, 25° C., 3 weeks | 98.9 | 1.1 | 0.09 |

Abbreviations: ND = not detected
^{a} Method PQL = 0.05%

TABLE 18
cIEF Results for H2O2 Treated Dostarlimab

| | pI of Main | Acidic | Main | Basic |
|---|---|---|---|---|
| Condition | Peak | Peaks % | Peak % | Peaks % ^{a} |
| Unstressed Control | 7.50 | 21.8 | 73.9 | <5.0 |
| T0 (exposed to H₂O₂, | 7.50 | 24.5 | 71.3 | <5.0 |
| no incubation) | | | | |
| H₂O₂, 25° C., 1 day | 7.48 | 24.1 | 71.3 | <5.0 |
| H₂O₂, 25° C., 3 days | 7.48 | 24.7 | 71.0 | <5.0 |
| H₂O₂, 25° C., 1 week | 7.48 | 27.9 | 67.9 | <5.0 |
| H₂O₂, 25° C., 2 weeks | 7.48 | 33.5 | 62.6 | <5.0 |
| H₂O₂, 25° C., 3 weeks | 7.48 | 39.1 | 57.2 | <5.0 |

^{a} Method PQL = 5.0%

TABLE 19
PTM Abundance in H₂O₂ Treated Dostarlimab

| | | | PTM Abundance (%) ^{a} | | | |
|---|---|---|---|---|---|---|
| | | | | | H₂O₂, | H₂O₂, |
| | | | Unstressed | | 25° C., | 25° C., |
| Peptide | Region | PTM | Control | T0 | 1 w | 2 w |
| LH: E1-K43 | CDR | HC: Met 34 Oxidation | <1.0 | <1.0 | 14.0 | 28.8 |
| TH: N77-R87 | Fab, Non-CDR | HC: Met 83 Oxidation | <1.0 | <1.0 | <1.0 | 1.1 |
| TH: A88-K120 | CDR | HC: Met 103 Oxidation | <1.0 | 1.2 | 64.9 | 86.1 |
| LH: D245-K284 | Fc | HC: Met 248 Oxidation | 3.0 | 47.1 | 98.6 | 99.6 |
| TH: T252-K284 | Fc | HC: Asp 261/266/276 | 1.0 | <1.0 | <1.0 | <1.0 |
| | | Isomerization ^{b} | | | | |
| TH: V298-K313 | Fc | HC: Asn 311 | 6.0 | 6.8 | 7.0 | 7.4 |
| | | Deamidation | | | | |
| LH: G337-K356 | Fc | HC: Met 354 Oxidation | <1.0 | 16.7 | 99.8 | 99.8 |
| LH: G367-K388 | Fc | HC: Asn 380 | 8.4 | 10.7 | 10.9 | 10.5 |
| | | Deamidation | | | | |
| LH: G367-K388 | Fc | HC: Asn 385 | <1.0 | 1.7 | 1.8 | 1.9 |
| | | Deamidation | | | | |
| LH: S411-K435 | Fc | HC: Met 424 Oxidation | <1.0 | 29.0 | 98.0 | 98.1 |
| LH: S411-K435 | Fc | HC: Asn 430 | 1.2 | <1.0 | <1.0 | <1.0 |
| | | Deamidation | | | | |
| LH: S436-K443 | C-terminal | HC: Lys 443 Clipped | 97.8 | 97.2 | 98.0 | 97.7 |
| TL: L46-R61 | CDR | LC: Trp 50 Oxidation | <1.0 | <1.0 | <1.0 | 1.3 |
| LL: S127-K145 | Fab, Non-CDR | LC: Asn 137 | <1.0 | 1.3 | 1.3 | 1.3 |
| | | Deamidation | | | | |

LH: Heavy chain peptide from Lys-C digest,
TH: Heavy Chain peptide from tryptic digest,
LL: Light chain peptide from Lys-C digest,
TL: Light chain peptide from tryptic digest
^{a} PTM levels were not listed when they were <1.0% in all samples
^{b} The modification site was not confirmed by MS/MS

TABLE 20
Relative Potency Results (MSD Binding, Cell-Based Bioassay
and FcRn Binding) for H₂O₂ Treated Dostarlimab

| | Relative Potency | | FcRn |
|---|---|---|---|
| | MSD | Cell-Based | Binding |
| Condition | Binding | Bioassay | (KD, M) |
| Unstressed Control | 88% | 98% | 2.14 × 10⁻⁶ |
| T0 (exposed to H₂O₂, | 95% | 94% | 4.38 × 10⁻⁶ |
| no incubation) | | | |
| H₂O₂, 25° C., 1 week | 79% | NT | NT |
| H₂O₂, 25° C., 2 weeks | 74% | 47% | 1.24 × 10⁻⁵ |

Abbreviations: NT = not tested

In summary, the degradation products observed under H₂O₂ oxidative conditions are fragments, oxidation in Fc region (Met 248, Met 354 and Met 424) and CDR (Met 34 and Met 103). The combination of attributes below has no negative impact to relative potency or FcRn binding:
Fragments of 0.5-1.0% (from LMW %+MMW % reduced CE-SDS and pre-peak % in non-reduced CE-SDS)
Oxidation of the Fc region of the HC: Met 248 up to 47.1%, Met 354 up to 16.7% and Met 424 up to 29.0% (from peptide map)
Oxidation of the CDR region of the HC: Met 34 less than 1.0% and Met 103 up to 1.2% (from peptide map).

Extrapolating from the data for oxidation at HC Met 34 and potency (bioassay), up to 21% oxidation can result in at least 60% potency (bioassay). Up to 16% oxidation at Met 34 can result in at least 70% potency (bioassay). This is calculated using a linear slope for <1% (control), <1% (T0) and 28.8% (H₂O₂ at 2 weeks) oxidation samples, which have 98%, 94% and 47% potency (bioassay), respectively.

Extrapolating from the data for oxidation at HC Met 103 and potency (bioassay), up to 64% oxidation can result in at least 60% potency (bioassay). Up to 47% oxidation at Met 103 can result in at least 70% potency (bioassay). This is calculated using a linear slope for <1% (control), 1.2% (T0) and 86.1% (H₂O₂ at 2 weeks) oxidation samples, which have 98%, 94% and 47% potency (bioassay), respectively.

It is expected that oxidation of the Fc region of the HC can go higher than the reported levels of: Met 248 up to 47.1%, Met 354 up to 16.7% and Met 424 up to 29.0%, without any impact to relative potency or FcRn binding.

### 5.3.2 AAPH Treated

Since H₂O₂ treated dostarlimab did not show oxidation of Trp residues in the CDR region, 2,2′-Azobis(2-amidinopropane) dihydrochloride (AAPH) was used to evaluate the impact of Trp oxidation. AAPH was spiked into dostarlimab samples to reach final concentration of 5 mM AAPH. The AAPH spiked samples were incubated at 40° C. for up to 7 days. The 40° C. condition was chosen based on initial condition screening to ensure a meaningful level of degradation can be observed. Upon completion, AAPH treatment was quenched with methionine and samples were kept frozen until the time of testing. The T0 sample was exposed to the AAPH without incubation at the elevated temperature. Methionine was added to sample immediately after AAPH addition to quench the reaction and arrest degradation.

The SE-HPLC results showed an increase in HMW species in AAPH treated samples starting from 1 day of incubation. The LMW species was slightly elevated, but remained less than 1% during the 7 day incubation period (Table 21).

The cIEF data showed a decrease in the main peak percentage starting from 1 day incubation and the trend continued as incubation time extended (Table 22). As the main peak decreased, both acidic species and basic species increased. Furthermore, the main peak in cIEF shifted slightly to the acidic side as well. Both reduced and non-reduced CE-SDS data indicated increased HMW species and fragments (not shown); The changes emerged at day 1 in the non-reduced CE-SDS data and on day 3 in the reduced CE-SDS data. The peptide map data indicated elevated oxidation at HC Met 248, HC Met 354, HC Met 424 and LC Trp 50 starting from day 1. The oxidation level at Met 103 also showed a slightly increasing trend at day 1, which was confirmed by the increase seen on day 3 data (Table 23). All other PTM levels were within analytical variability.

The cIEF of the AAPH treated samples showed a change in the shape of the main peak and the charge profile shifted to more acidic species. This demonstrated that oxidized species are located in acidic peak region and cIEF is capable of monitoring for excessive oxidation in dostarlimab.

Starting from the 3-day incubation sample, the relative potency (MSD binding assay) was decreased by the changes observed in AAPH treated samples (Table 24). The relative potency started to decrease by the cell based bioassay after day 1 of incubation and by the MSD binding assay after day 3 of incubation. Like the T0 H₂O₂ sample, immediate exposure to AAPH (T0) did not induce a potency drop in dostarlimab sample; both T0 samples having low levels of oxidation in the CDR. The exception being, the level of Fc oxidation was much higher in the H₂O₂ T0 sample. This finding substantiates that increased Fc oxidation does not impact potency.

The AAPH day 1 sample had comparable methionine oxidation to the T0 H₂O₂ sample in both the CDR and Fc region. However, the AAPH day 1 sample showed a decrease in potency. The main difference between the T0 H₂O₂ and the day1 AAPH treated sample was the level of CDR LC Trp 50 oxidation. Therefore, an increase in CDR Trp 50 oxidation will decrease relative potency. The comparison of potency results from peroxide and AAPH treated samples is provided in Table 25.

Starting from the 3-day AAPH incubation sample, FcRn binding was reduced. Therefore, FcRn binding decreases with increasing oxidation which is consistent with the H₂O₂ treated sample finding. After 3 days, Met in the CDR region (HC Met 103 at ˜7%) and Fc region (Met 248 at˜89%, Met 354 at ˜45% and Met 424 at ˜78%) were excessively oxidized. The reduced FcRn binding is likely due to oxidation in the Fc region which is involved with FcRn binding.

TABLE 21
SE-HPLC Results for AAPH Treated Dostarlimab

| Condition | Monomer % | HMW % | LMW % |
|---|---|---|---|
| Unstressed Control | 99.1 | 0.9 | ND |
| T0 (exposed to AAPH, | 99.1 | 0.9 | ND |
| no incubation) | | | |
| AAPH, 40° C., 1 day | 94.9 | 4.9 | 0.2 |
| AAPH, 40° C., 3 days | 87.8 | 11.8 | 0.5 |
| AAPH, 40° C., 5 days | 82.8 | 16.4 | 0.8 |
| AAPH, 40° C., 7 days | 80.4 | 18.7 | 0.9 |

Abbreviations: ND = not detected

TABLE 22
cIEF Results for AAPH Treated Dostarlimab

| | pI of Main | Acidic | Main | Basic |
|---|---|---|---|---|
| Condition | Peak | Peaks % | Peak % | Peaks % ^{a} |
| Unstressed Control | 7.50 | 21.8 | 73.9 | <5.0 |
| T0 (exposed to AAPH, | 7.51 | 22.4 | 73.4 | <5.0 |
| no incubation) | | | | |
| AAPH, 40° C., 1 day | 7.50 | 25.6 | 66.7 | 7.7 |
| AAPH, 40° C., 3 days | 7.49 | 30.7 | 61.5 | 7.9 |
| AAPH, 40° C., 5 days | 7.48 | 38.3 | 51.0 | 10.7 |
| AAPH, 40° C., 7 days | 7.48 | 42.5 | 46.6 | 10.9 |

^{a} Method PQL = 5.0%

TABLE 23
PTM Abundance in AAPH Treated Dostarlimab

| | | | PTM Abundance (%) ^{a} | | | |
|---|---|---|---|---|---|---|
| | | | | | AAPH, | AAPH, |
| | | | Unstressed | | 40° C., | 40° C., |
| Peptide | Region | PTM | Control | T0 | 1 day | 3 day |
| TH: A88-K120 | CDR | HC: Met 103 Oxidation | <1.0 | <1.0 | 2.5 | 7.0 |
| LH: D245-K284 | Fc | HC: Met 248 Oxidation | 3.0 | 3.3 | 47.7 | 88.4 |
| TH: T252-K284 | Fc | HC: Asp 261/266/276 | 1.0 | 1.0 | <1.0 | 1.0 |
| | | Isomerization ^{b} | | | | |
| TH: V298-K313 | Fc | HC: Asn 311 | 6.0 | 6.3 | 6.5 | 6.6 |
| | | Deamidation | | | | |
| LH: G337-K356 | Fc | HC: Met 354 Oxidation | <1.0 | <1.0 | 8.2 | 44.8 |
| LH: G367-K388 | Fc | HC: Asn 380 | 8.4 | 9.5 | 8.6 | 9.6 |
| | | Deamidation | | | | |
| LH: G367-K388 | Fc | HC: Asn 385 | <1.0 | 1.0 | 1.3 | 1.2 |
| | | Deamidation | | | | |
| LH: S411-K435 | Fc | HC: Met 424 Oxidation | <1.0 | <1.0 | 26.2 | 78.2 |
| LH: S411-K435 | Fc | HC: Asn 430 | 1.2 | 1.3 | 1.1 | <1.0 |
| | | Deamidation | | | | |
| LH: S436-K443 | C-terminal | HC: Lys 443 Clipped | 97.8 | 97.8 | 96.7 | 97.7 |
| TL: L46-R61 | CDR | LC: Trp 50 Oxidation | <1.0 | <1.0 | 38.9 | 74.2 |
| LL: S127-K145 | Fab, Non-CDR | LC: Asn 137 | <1.0 | 1.0 | 1.2 | <1.0 |
| | | Deamidation | | | | |

Abbreviations:
LH = Heavy chain peptide from Lys-C digest,
LL = Light chain peptide from Lys-C digest,
TH = Heavy Chain peptide from tryptic digest,
TL = Light chain peptide from tryptic digest
^{a} PTM levels were not listed when they were <1.0% in all samples
^{b} The modification site was not confirmed by MS/MS

TABLE 24
Relative Potency Results (MSD Binding, Cell-Based Bioassay
and FcRn Binding) for AAPH Treated Dostarlimab

| | Relative Potency | | FcRn |
|---|---|---|---|
| | MSD | Cell-Based | Binding |
| Condition | Binding | Bioassay | (KD, M) |
| Unstressed Control | 88% | 98% | 2.14 × 10⁻⁶ |
| T0 (exposed to AAPH, | 79% | 102% | 1.66 × 10⁻⁶ |
| no incubation) | | | |
| AAPH, 40° C., 1 day | 76% | 44% | 2.34 × 10⁻⁶ |
| AAPH, 40° C., 3 days | 57% | 14% | 8.33 × 10⁻⁶ |
| AAPH, 40° C., 5 days | 47% | 5% | NT |
| AAPH, 40° C., 7 days | 43% | NT | 9.69 × 10⁻⁶ |

Abbreviations: NT = Not tested

TABLE 25
Impact of Site Specific Oxidation on
the Relative Potency of Dostarlimab

| Molecular | | Percent Oxidation | | |
|---|---|---|---|---|
| Region | Residue | H₂O₂ T0 | AAPH T0 | AAPH Day 1 |
| CDR | HC: Met 34 | low | low | low |
| | HC: Met 103 | low | low | low |
| | LC: Trp 50 | low | low | high |
| Fc | HC: Met 248 | high | low | high |
| | HC: Met 354 | high | low | high |
| | HC: Met 424 | high | low | high |

| | | | |
|---|---|---|---|
| Potency by Cell-Based | No Impact | No Impact | Reduced |
| Bioassay | | | |

In summary, the degradation products observed under AAPH oxidative conditions are HMW species, fragments, oxidation in Fc region Met (HO Met 248, HO Met 354 and HO Met 424), CDR region Met (HO Met 103) and LC CDR Trp 50. The combination of attributes below has no negative impact to relative potency and FcRn binding:
HMW species up to 0.9% (from SE-HPLC);
Fragments 0.8-1.2% (from LMW %+MMW % reduced CE-SDS and pre-peak % in non-reduced CE-SDS);
Oxidation in Fc region HO Met 248 up to 3.3%, HO Met 354 less than 1.0% and HO Met 424 less than 1.0% (from peptide map);
Oxidation in CDR region HO Met 103 less than 1.0%, LC Trp 50 less than 1.0% (from peptide map).

Extrapolating from the data for oxidation at LC Trp 50 and potency (bioassay), up to 34% oxidation can result in at least 60% potency (bioassay). Up to 25% oxidation at Trp 50 can result in at least 70% potency (bioassay). This is calculated using a linear slope for <1% (control), <1% (T0), 38.9% (1 day), 74.3% (3 day) and 86.8% (5 day) oxidation samples, which have 98%, 102%, 44%, 14% and 5% potency (bioassay), respectively.

Since oxidation of LC Trp 50 is the dominant oxidized variant under AAPH oxidative conditions, the H₂O₂ oxidative conditions are more reflective of permissive oxidation levels in CDR region HC Met 34, HC Met 103 and the Fc region HC Met 248, HC Met 354 and HC Met 424.

### 5.4 Photolysis

The dostarlimab samples were exposed to white light (visible light, 10 000 lux hours) for 1 week at 25° C. and then exposed to UV light for 1 week at 25° C. Upon completion, samples were kept frozen until the time of testing.

The SE-HPLC results showed an increase in HMW species at the end of photolytic conditions (Table 26). The cIEF data showed an increase in acidic species percentage and decreased main peak percentage (Table 27). The reduced CE-SDS results showed no changes outside analytical variability. The non-reduced CE-SDS data indicated a decrease in main peak and slight increase in both fragments and HMW species.

The peptide map data showed elevated oxidation in Fc region Met (HC Met 248, HC Met 354 and HC Met 424) and LC CDR Trp 50 at the end of the photolysis study (Table 28). All other PTM levels were within analytical variability.

The relative potency and FcRn binding were not impacted by the photolytic conditions (Table 29). Therefore, the observed moderate oxidation level in Fc region Met (HC Met 248<33%, HC Met 354<11%, HC Met 424<27%) and LC CDR Trp 50 (<5%) had no impact to relative potency and FcRn binding.

TABLE 26
SE-HPLC Results for Photo Degraded Dostarlimab

| Condition | Monomer % | HMW % | LMW % ^{a} |
|---|---|---|---|
| Unstressed Control | 99.1 | 0.9 | ND |
| Vis light, 25° C., 1 week | 97.8 | 2.2 | <0.05 |
| Vis light, 25° C., 1 week and | 96.6 | 3.3 | <0.05 |
| UV light, 25° C., 1 week | | | |

ND: not detected
^{a} Method PQL = 0.05%

TABLE 27
cIEF Results for Photo Degraded Dostarlimab

| | pI of | | | |
|---|---|---|---|---|
| | Main | Acidic | Main | Basic |
| Condition | Peak | Peaks % | Peak % | Peaks % ^{a} |
| Unstressed Control | 7.50 | 21.8 | 73.9 | <5.0 |
| Vis light, 25° C., 1 week | 7.51 | 23.9 | 71.7 | <5.0 |
| Vis light, 25° C., 1 week and | 7.50 | 28.5 | 66.9 | <5.0 |
| UV light, 25° C., 1 week | | | | |

^{a} Method PQL = 5.0%

TABLE 28
PTM Abundance in Photo Degraded Dostarlimab

| | | | PTM Abundance (%) ^{a} | |
|---|---|---|---|---|
| | | | | Vis light, 25° C., |
| | | | | 1 week and UV |
| | | | Unstressed | light, 25° C., |
| Peptide | Region | PTM | Control | 1 week |
| LH: D245- | Fc | HC: Met 248 | 3.0 | 33.4 |
| K284 | | Oxidation | | |
| TH: T252- | Fc | HC: Asp 261/ | 1.0 | 1.0 |
| K284 | | 266/276 | | |
| | | Isomerization ^{b} | | |
| TH: V298- | Fc | HC: Asn 311 | 6.0 | 6.4 |
| K313 | | Deamidation | | |
| LH: G337- | Fc | HC: Met 354 | <1.0 | 10.9 |
| K356 | | Oxidation | | |
| LH: G367- | Fc | HC: Asn 380 | 8.4 | 9.5 |
| K388 | | Deamidation | | |
| LH: G367- | Fc | HC: Asn 385 | <1.0 | 1.3 |
| K388 | | Deamidation | | |
| LH: S411- | Fc | HC: Met 424 | <1.0 | 27.0 |
| K435 | | Oxidation | | |
| LH: S411- | Fc | HC: Asn 430 | 1.2 | 1.2 |
| K435 | | Deamidation | | |
| LH: S436- | C- | HC: Lys 443 | 97.8 | 97.8 |
| K443 | terminal | Clipped | | |
| TL: L46- | CDR | LC: Trp 50 | <1.0 | 4.8 |
| R61 | | Oxidation | | |

Abbreviations: LH = Heavy chain peptide from Lys-C digest, TH = Heavy Chain peptide from tryptic digest, LL = Light chain peptide from Lys-C digest, TL = Light chain peptide from tryptic digest
^{a} PTM levels were not listed when they were <1.0% in all samples
^{b} The modification site was not confirmed by MS/MS

TABLE 29
Relative Potency Results (MSD Binding, Cell-Based Bioassay
and FcRn Binding) for Photo Degraded Dostarlimab

| | Relative Potency | | FcRn |
|---|---|---|---|
| | MSD | Cell-Based | Binding |
| Condition | Binding | Bioassay | (KD, M) |
| Unstressed Control | 88% | 98% | 2.14 × 10⁻⁶ |
| Vis light, 25° C., 1 week and | 92% | 90% | 2.49 × 10⁻⁶ |
| UV light, 25° C., 1 week | | | |

In summary, the degradation products observed under photolysis conditions are HMW species, fragments, oxidation in Fc region Met (Met 248, Met 354 and Met 424) and LC CDR Trp 50. The combination of attributes below has no negative impact to relative potency and FcRn binding:
HMW species up to 3.3% (from SE-HPLC)
Fragments 1.1-1.5% (from LMW %+MMW % reduced CE-SDS and pre-peak % in non-reduced CE-SDS)
Oxidation in Fc region HC Met 248 up to 33.4%, HC Met 354 up to 10.9% and Met 424 up to 27.0% (from peptide map)
Oxidation in CDR LC Trp 50 up to 4.8% (from peptide map)

### 5.5 Sustained Agitation

The dostarlimab samples were exposed to agitation at 300 revolutions per minute (rpm) for up to 3 weeks at 25° C. Upon completion, samples were kept frozen until the time of testing.

No product quality changes in SE-HPLC, cIEF, reduced CE-SDS and non-reduced CE-SDS were observed.

The relative potency and FcRn binding were not impacted by the sustained agitation. The peptide map data showed all PTM levels were within analytical variability.

In summary, under sustained agitation conditions, no significant degradant was observed and no impact to relative potency and FcRn binding was found.

### 5.6 Degradation Product Summary

The prevalence of degradation products observed under different forced degradation conditions which have no impact on dostarlimab potency are summarized below (Table 30).

TABLE 30
Degradation Products which do not Impact the Potency of Dostarlimab

| | Level of Degradants Observed Having no Impact on Potency | | | | | |
|---|---|---|---|---|---|---|
| | | | | | | Relative |
| Condition | HMW | Fragments | Isomerization | Deamidation | Oxidation | Potency ^{a} |
| Unstressed | 0.9% | 0.6-1.0% | HC Asp | HC Asn | HC Met | 98% |
| | | | 261/266/276 = 1.0% | 380 = 8.4% | 248 = 3.0% | |
| | | | | HC Asn | HC Met | |
| | | | | 385 < 1.0% | 354 < 1.0% | |
| | | | | | HC Met | |
| | | | | | 424 < 1.0% | |
| Thermal | ≤11.2% | 4.5-4.6% | HC Asp | HC Asn | HC Met | 86% |
| Degradation | | | 261/266/276 ≤ 13.1% | 380 ≤ 14.7% | 248 ≤ 7.6% | (50° C., |
| | | | | HC Asn | HC Met | 3 weeks) |
| | | | | 385 ≤ 5.9% | 354 ≤ 4.2% | |
| | | | | | HC Met | |
| | | | | | 424 ≤ 3.5% | |
| Acid | ≤15.2% | No Change | No Change | No Change | No Change | 125% |
| Hydrolysis | | | | | | (pH 4.0, |
| | | | | | | 3 weeks) |
| Base | ≤9.2% | 2.0-4.2% | HC Asp | HC Asn | HC Met | 96% |
| Hydrolysis | | | 147 ≤ 20.8% | 380 ≤ 27.8% | 248 ≤ 7.1% | (pH 9.0, |
| | | | LC Asp | HC Asn | | 1 week) |
| | | | 151/167 ≤ 3.1% | 385 ≤ 27.2% | | |
| Peroxide | No Change | 0.5-1.0% | No Change | No Change | HC Met | 94% |
| (H₂O₂) | | | | | 248 ≤ 47.1% | (H₂O₂, |
| Oxidation | | | | | HC Met | T0) |
| | | | | | 354 ≤ 16.7% | |
| | | | | | HC Met | |
| | | | | | 424 ≤ 29.0% | |
| | | | | | HC Met | |
| | | | | | 34 < 1.0% | |
| | | | | | HC Met | |
| | | | | | 103 ≤ 1.2% | |
| AAPH | ≤0.9% | 0.8-1.2% | No Change | No Change | HC Met | 102% |
| Oxidation | | | | | 248 ≤ 3.3% | (AAPH, |
| | | | | | HC Met | T0) |
| | | | | | 354 ≤ 1.0% | |
| | | | | | HC Met | |
| | | | | | 424 < 1.0% | |
| | | | | | HC Met | |
| | | | | | 103 < 1.0% | |
| | | | | | LC Trp | |
| | | | | | 50 < 1.0% | |
| Photolysis | ≤3.3% | 1.1-1.5% | No Change | No Change | HC Met | 90% |
| | | | | | 248 ≤ 33.4% | (Photolysis, |
| | | | | | HC Met | total |
| | | | | | 354 ≤ 10.9% | 2 weeks) |
| | | | | | HC Met | |
| | | | | | 424 ≤ 27.0% | |
| | | | | | LC Trp | |
| | | | | | 50 ≤ 4.8% | |
| Sustained | No Change | No Change | No Change | No Change | No Change | 86% |
| Agitation | | | | | | (Agitation, |
| | | | | | | 3 weeks) |

^{a} Potency by cell-based bioassay

### 6. Formulation Studies

### 6.1 Analytical Methods

6.1.1 Appearance

The appearance of samples, including clarity and color were inspected under black and white background using YB-2 light box.

6.1.2 pH

The pH value was measured by Seven Multi pH Meter.

6.1.3 UV 280

Protein concentration was determined by absorbance at 280 nm using NANODROP2000 spectrophotometer, the extinction coefficient is 1.615. The samples were diluted gravimetrically.

6.1.4 SEC-HPLC

Size exclusion chromatography was performed using an Agilent 1260 Infinity system and a TSKGel G3000SWXL column (300×7.8 mm, 5 μm). The mobile phase was 50 mM phosphate buffer (PB), 300 mM NaCl, pH 7.0±0.1 and the flow rate was set as 1.0 ml/min. Samples were diluted to 1 mg/ml for injection and detected at 280 nm.

6.1.5 cIEF

20 μg of the reference standard or sample was mixed with 0.5 μl of pI 5.85 marker, 0.5 μl of pI 8.40 marker, 2 μl of Pharmalyte 3-10, 2 μl of Pharmalyte 5-8, 35 μl of 1% methyl cellulose (MC), and purified water to make up final volume of 100 μl. The mixture was then analyzed with iCE3 Capillary Isoelectric Focusing Analyzer equipped with a fluorocarbon (FC)-coated whole-column detection capillary. The focusing was carried out by two steps: (1) 1.5 kV for 1 min, and (2) 3 kV for 8 min. During the experiment, the autosampler tray was maintained at 5° C.

6.1.6 Dynamic Light Scattering (DLS)

A micropipette was used to transfer an aliquot of 40 μl of undiluted sample to a 40 μl disposable cuvette in a safety hood. Triplet measurements were performed for each sample. Data were auto-analyzed by Zetasizer Software.

6.1.7 DSC

Capillary cell differential scanning calorimetry (DSC) is utilized to measure the thermal stability of proteins by detecting the difference in the amount of heat required to increase the temperature of a sample and reference as a function of temperature. Specifically, it is used to measure the thermal transition midpoint (Tm), which is an indicator of the relative stability of protein in solution. Samples were diluted into about 1 mg/ml with reference buffer. 400 μl of reference buffers were added into the odd-numbered wells of 96-well plate, and 400 μl of samples were added into the even-numbered wells of 96-well plate. The samples were scanned from 20-110° C. and data analysis was performed in MicroCal VPCapillary DSC Automated data analysis software.

6.2 pH/Buffer Screening Study

Four types of buffer systems each at three different pHs were prepared, these were 25 mM acetate buffer pH 4.5, 25 mM acetate buffer pH 5.0, 25 mM acetate buffer pH 5.5, citrate buffer pH5.0, 25 mM citrate buffer pH 5.5, 25 mM citrate buffer pH 6.0, 25 mM histidine buffer pH 5.5, 25 mM histidine buffer pH 6.0, 25 mM histidine buffer pH 6.5, 25 mM phosphate buffer pH 6.5, 25 mM phosphate buffer pH 7.0, and 25 mM phosphate buffer pH 7.5 (summarized in Table 31).

TABLE 31
Summary of Buffer Systems Tested

| Candidate | Buffer | pH |
|---|---|---|
| 1 | 25 mM acetate | 4.5 |
| 2 | | 5.0 |
| 3 | | 5.5 |
| 4 | 25 mM citrate | 5.0 |
| 5 | | 5.5 |
| 6 | | 6.0 |
| 7 | 25 mM histidine | 6.0 |
| 8 | | 6.5 |
| 9 | | 7.0 |
| 10 | 25 mM phosphate | 6.5 |
| 11 | | 7.0 |
| 12 | | 7.5 |

Bulk drug substance was buffer-exchanged into the 12 buffer systems of Table 21 via centrifugal diafiltration. Sample protein concentrations were subsequently adjusted to 20 mg/ml with corresponding buffers. Each sample was then sterile filtered and filled into glass vials and stoppered and capped immediately.

Samples were stored still at 40° C. and agitated at 30° C. at 250 rpm for up to 4 weeks. The samples were tested for appearance, pH, UV280, SEC-HPLC, cIEF, DLS, and particulate matter.

All of the samples stayed clear and colorless or slightly opalescent and colorless when stored at 40° C. for 4 weeks and shaken at 250 rpm, 30° C. for 2 weeks.

The pH values of all the 12 buffer systems seemed stable during agitation and storage at 40° C.

The protein concentrations of all samples were stable around 20 mg/ml.

The particulate matter counts of all the 12 buffer systems were stable during agitation and storage at 40° C.

SEC-HPLC results: When stored at 40° C. for 4 weeks, candidates 1, 4, 11 and 12 showed a faster speed of main peak contents decline, while candidates 5, 6, and 7 appeared to be the most stable. When agitated at 250 rpm, 30° C. for 4 weeks, candidates 10, 11, and 12 showed a faster speed of main peak decline, while the candidates 7 and 8 appeared to be the most stable. When stored at 40° C. for 4 weeks, candidates 4, 8, 9, 10, 11 and 12 showed a faster speed of acidic peak formation; when agitated with 250 rpm at 30° C. for 4 weeks, candidates 9, 11, and 12 showed a faster speed of acidic peak formation.

DLS results: Candidates 1, 2 and 4 showed a shift in hydrodynamic radius (Z-average) when samples were stored at 40° C. for 4 weeks. This indicates the protein is likely unstable in the buffer systems and changes in folding and/or surface potential might have caused changes in inter-molecular interactions which in term led to different association or aggregation patterns as observed in DLS readings. Candidates 2, 3 and 12 showed the same unstable performance in Z-average when samples were agitated with 250 rpm at 30° C. for 4 weeks. The polydispersity index (PDI) value is another index that reflects sample's stability performance by DLS. Candidates 6, 7 and 10 kept reasonably small PDI values during the study while Candidates 1, 2, 3, 4 and 12 showed unstable and relatively bigger PDI values.

Thus the results indicate that citrate buffer at pH 5.5 or 6.0 and histidine buffer at pH 6.0 or 6.5 are the most stable buffer systems.

### 6.3 Excipient Screening

Preferred buffer systems were tested with a range of excipients (different candidate formulations summarized in Table 32).

TABLE 32
Summary of Excipients Screened

| | | | Excipient | | | | | |
|---|---|---|---|---|---|---|---|---|
| | Buffer | PS80 | | Sucrose | Trehalose | | | Methionine |
| Candidate | and pH | (w/v) | Sorbitol | (w/v) | (w/v) | Arginine | NaCl | (w/v) |
| F1 | 25 mM | 0.02% | — | — | — | — | 120 mM | — |
| F2 | citrate | | — | — | — | 100 mM | — | — |
| F3 | pH 6.0 | | — | — | 5% | — | — | — |
| F4 | | | — | 5% | — | — | — | — |
| F5 | | | 100 mM | — | — | — | — | — |
| F6 | 25 mM | 0.02% | — | — | — | — | 120 mM | — |
| F7 | histidine | | — | — | — | 100 mM | — | — |
| F8 | pH 6.0 | | — | — | 5% | — | — | — |
| F9 | | | — | 5% | — | — | — | — |
| F10 | | | 100 mM | — | — | — | — | — |
| F11 | | | — | 5% | — | — | — | 0.05% |

Note:
Osmolality of each formulation adjusted to meet 290 mOsm/kg by adding NaCl if needed.

Samples were stored still at 40° C. and agitated at 30° C. at 250 rpm for up to 4 weeks. The samples were tested for appearance, pH, UV280, SEC-HPLC, cIEF, DLS, and particulate matter.

All of the samples stayed slightly opalescent and colorless liquid when stored at 40° C. for 4 weeks and shaken with 250 rpm at 30° C. for 2 weeks.

The pH values and osmolality of all the 11 formulations (F1-F11) seemed stable during agitation and storage at 40° C.

The protein concentrations of all samples were stable around 20 mg/ml.

The particulate matter counts of all the 11 formulations were stable during agitation and storage at 40° C.

SEC-HPLC results: When stored at 40° C. for 4 weeks and agitated with 250 rpm at 30° C. for 4 weeks, candidate F2 seemed to be the most stable formulation among the 11 candidates.

cIEF results: When stored at 40° C. for 4 weeks and agitated with 250 rpm at 30° C. for 4 weeks, candidates F2 and F3 seemed to be the most stable formulations among the 11 candidates.

All the 11 candidates kept stable hydrodynamic radius (Z-average) when samples were stored at 40° C. for 4 weeks and agitated with 250 rpm at 30° C. for 4 weeks.

Except candidates 4 and 11, all the other candidates kept reasonably small PDI values during the study.

### 6.4 High Concentration Study

Dostarlimab is currently formulated at 20 or 50 mg/mL in 25 mM citrate, 100 mM L-Arginine-HCl, 31 mM NaCl, 0.02% (w/v) PS80, at pH 6.0. A study concentrating the antibody concentration up to 125 mg/mL was performed to evaluate the stability of the protein in high concentration formulations. Five protein concentrations were evaluated in the study, ranging from 50-125 mg/mL in increments of 25 mg/mL protein concentration. The study results indicated that dostarlimab could be concentrated to 125 mg/mL and was stable in general.

The antibody is formulated at the Drug Substance stage. Since the Drug Product (DP) is the same strength and formulation as the Drug Substance (DS), the forced degradation studies described above are applicable to the Drug Product.

### 6.5 Shelf Life Stability

The dostarlimab 50 mg/mL drug product (DP) is stable in the formulation selected under the recommended long-term storage conditions. Conclusions can be based on the studies performed:
No changes in product quality have been observed for up to 18 months at the long-term

stability storage condition of 5° C.;
Only minor changes in product quality were observed for all the drug product batches

after 6 months of storage at the accelerated condition of 25±5° C.;
The dostarlimab drug product demonstrates photostability equivalent to 30 days at

ambient light conditions.

Results obtained from the stability program to date indicate that dostarlimab DP is stable when stored at the recommended long-term storage condition of (5±3° C.), no discernable changes are seen when evaluated through analytical testing for up to 18 months of storage. Trend analysis of the DP stored at the accelerated condition (25±2° C./60±5% Relative Humidity (RH)) for up to 6 months shows only minor changes to product quality attributes. At this time, the real-time, real-temperature data substantiate the recommended long-term storage of the drug product at 5±3° C. for up to 18 months, but stability data will continue to be collected and evaluated to further extend the shelf life as appropriate.

TABLE 33
Dostarlimab DP Stability Specifications

| | | Acceptance Criteria for |
|---|---|---|
| Test/Attribute | Analytical Methodology | Stability Testing^{a} |
| pH | Potentiometric: | 5.5-6.5 |
| | Ph. Eur. 2.2.3, USP <791> | |
| Purity | CE-SDS, non-reduced | Main Peak: ≥95.0% |
| Purity | SE-HPLC | Monomer: ≥95.0% |
| | | HMW species: ≤4.0% |
| | | LMW species: ≤1.0% |
| Charge | cIEF | Main peak: ≥55.0% |
| heterogeneity | | Acidic species: ≤35.0% |
| | | Basic species: ≤15.0% |
| Potency: | MSD binding assay^{b} | 60%-140% of reference |
| Inhibition | | material activity |
| of PDL1 | | |
| binding | | |
| Protein | UV absorbance at 280 nm | 45-55 mg/mL |
| Concentration | | |

^{a}Tests and acceptance criteria applicable to the clinical drug product batches prior to registration. Additional tests may have been run historically and may be run in the future per special protocol for comparability or other purposes.
^{b}MSD binding assay will continue to be used for ongoing stability batches that were initiated using that assay

The invention includes the following items:

1. A composition comprising an oxidized variant of an anti-PD-1 antibody, wherein the oxidized variant comprises a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises ≤65%, ≤60%, ≤50%, ≤40%, ≤30%, ≤20%, ≤15%, ≤10%, ≤5%, ≤4%, or ≤3% of oxidized variant.

2. The composition according to item 1, wherein the amount of oxidized variant in the composition is between the lower limit of detection of the method used to identify the oxidized variant and 65%, 60%, 50%, 40%, 30%, 20%, 15%, 10%, 5%, 4% or 3%.

3. The composition according to item 1 or item 2, comprising the oxidized variant in an amount:
(i) selected from any one of the following ranges: 0.01-65%, 0.01-60%, 0.01-50%, 0.01-40%, 0.01-30%, 0.01-20%, 0.01-15%, 0.01-10%, 0.01-5%, 0.01-4%, 0.01-3%, 0.05-65%, 0.05-60%, 0.05-50%, 0.05-40%, 0.05-30%, 0.05-20%, 0.05-15%, 0.05-10%, 0.05-5%, 0.05-4%, 0.05-3%, 0.1-65%, 0.1-60%, 0.1-50%, 0.1-40%, 0.1-30%, 0.1-20%, 0.1-15%, 0.1-10%, 0.1-5%, 0.1-4%, 0.1-3%, 0.5-65%, 0.5-60%, 0.5-50%, 0.5-40%, 0.5-30%, 0.5-20%, 0.5-15%, 0.5-10%, 0.5-5%, 0.5-4%, 0.5-3%, 1-65%, 1-60%, 1-50%, 1-40%, 1-30%, 1-20%, 1-15%, 1-10%, 1-5%, 1-4%, 1-3%, 2-4%, and 2-3%; or
(ii) about 10%, about 5%, about 4%, about 3%, about 2%, or about 1%.

4. The composition according to any one of the preceding items, wherein the oxidized variant comprises oxidation at a methionine and/or tryptophan residue in any one of SEQ ID NOs: 1-6.

5. The composition according to any one of the preceding items, wherein the oxidized variant comprises one or a combination of oxidation at: M34 of CDRH1, M103 of CDRH3 and/or W50 of CDRL2.

6. The composition according to any one of the preceding items, wherein the composition comprises:

(i) ≤21%, ≤20%, ≤16%, ≤15%, ≤12.5%, ≤10%, ≤7.5%, ≤5%, ≤4%, ≤3%, ≤2%, or 51% oxidation at M34 of CDRH1;

(ii) ≤64%, ≤60%, ≤50%, ≤47%, ≤40%, ≤30%, ≤20%, ≤15%, ≤10%, ≤5%, ≤2%, or ≤1% oxidation at M103 of CDRH3; and/or

(iii) ≤30%, ≤25%, ≤20%, ≤15%, ≤10%, ≤7.5%, ≤5%, ≤4%, ≤3%, ≤2%, or ≤1% oxidation at W50 of CDRL2.

7. The composition according to any one of the preceding items, wherein the composition comprises oxidation at M34 of CDRH1 in an amount that is between the lower limit of detection of the method used to identify oxidation at M34 of CDRH1 and 21%, 20%, 16%, 15%, 12.5%, 10%, 7.5%, 5%, 4%, 3%, 2%, or 1%.

8. The composition according to any one of the preceding items, wherein the composition comprises oxidation at M34 of CDRH1 in an amount:
(i) selected from any one of the following ranges: 0.01-21%, 0.01-20%, 0.01-16%, 0.01-15%, 0.01-12.5%, 0.01-10%, 0.01-7.5%, 0.01-5%, 0.01-4%, 0.01-3%, 0.01-2%, 0.01-1%, 0.05-21%, 0.05-20%, 0.05-16%, 0.05-15%, 0.05-12.5%, 0.05-10%, 0.05-7.5%, 0.05-5%, 0.05-4%, 0.05-3%, 0.05-2%, 0.05-1%, 0.1-21%, 0.1-20%, 0.1-16%, 0.1-15%, 0.1-12.5%, 0.1-10%, 0.1-7.5%, 0.1-5%, 0.1-4%, 0.1-3%, 0.1-2%, 0.1-1%, 0.5-21%, 0.5-20%, 0.5-16%, 0.5-15%, 0.5-12.5%, 0.5-10%, 0.5-7.5%, 0.5-5%, 0.5-4%, 0.5-3%, 0.5-2%, and 0.5-1%; or
(ii) about 10%, about 5%, about 4%, about 3%, about 2%, or about 1%.

9. The composition according to any one of the preceding items, wherein the composition comprises oxidation at M103 of CDRH3 in an amount that is between the lower limit of detection of the method used to identify oxidation at M103 of CDRH3 and 64%, 60%, 50%, 47%, 40%, 30%, 20%, 15%, 10%, 5%, 2%, or 1%.

10. The composition according to any one of the preceding items, wherein the composition comprises oxidation at M103 of CDRH3 in an amount:

(i) selected from any one of the following ranges: 0.01-64%, 0.01-60%, 0.01-50%, 0.01-47%, 0.01-40%, 0.01-30%, 0.01-20%, 0.01-15%, 0.01-10%, 0.01-5%, 0.01-4%, 0.01-3%, 0.01-2%, 0.01-1%, 0.05-64%, 0.05-60%, 0.05-50%, 0.05-47%, 0.05-40%, 0.05-30%, 0.05-20%, 0.05-15%, 0.05-10%, 0.05-5%, 0.05-4%, 0.05-3%, 0.05-2%, 0.05-1%, 0.1-64%, 0.1-60%, 0.1-50%, 0.1-47%, 0.1-40%, 0.1-30%, 0.1-20%, 0.1-15%, 0.1-10%, 0.1-5%, 0.1-4%, 0.1-3%, 0.1-2%, 0.1-1%, 0.5-64%, 0.5-60%, 0.5-50%, 0.5-47%, 0.5-40%, 0.5-30%, 0.5-20%, 0.5-15%, 0.5-10%, 0.5-5%, 0.5-4%, 0.5-3%, 0.5-2% and 0.5-1%; or

(ii) about 10%, about 5%, about 4%, about 3%, about 2%, or about 1%.

11. The composition according to any one of the preceding items, wherein the composition comprises oxidation at W50 of CDRL2 in an amount that is between the lower limit of detection of the method used to identify oxidation at W50 of CDRL2 and 30%, 25%, 20%, 15%, 10%, 7.5%, 5%, 4%, 3%, 2%, or 1%.

12. The composition according to any one of the preceding items, wherein the composition comprises oxidation at W50 of CDRL2 in an amount:

(i) selected from any one of the following ranges: 0.01-34%, 0.01-30%, 0.01-25%, 0.01-20%, 0.01-15%, 0.01-10%, 0.01-7.5%, 0.01-5%, 0.01-4%, 0.01-3%, 0.01-2%, 0.01-1%, 0.05-34%, 0.05-30%, 0.05-25%, 0.05-20%, 0.05-15%, 0.05-10%, 0.05-7.5%, 0.05-5%, 0.05-4%, 0.05-3%, 0.05-2%, 0.05-1%, 0.1-34%, 0.1-30%, 0.1-25%, 0.1-20%, 0.1-15%, 0.1-10%, 0.1-7.5%, 0.1-5%, 0.1-4%, 0.1-3%, 0.1-2%, 0.1-1%, 0.5-34%, 0.5-30%, 0.5-25%, 0.5-20%, 0.5-15%, 0.5-10%, 0.5-7.5%, 0.5-5%, 0.5-4%, or 0.5-3%, 0.5-2% and 0.5-1%; or

(ii) about 10%, about 5%, about 4%, about 3%, about 2%, or about 1%.

13. The composition according to any one of the preceding items, wherein the antibody comprises a heavy chain variable region at least about 90% identical to the amino acid sequence of SEQ ID NO: 7 and/or a light chain variable region at least about 90% identical to the amino acid sequence of SEQ ID NO: 8.

14. The composition according to any one of the preceding items, wherein the antibody is at least about 90% identical to the heavy chain amino acid sequence of SEQ ID NO: 9 and/or at least about 90% identical to the light chain amino acid sequence of SEQ ID NO: 10.

15. The composition according to any one of the preceding items, wherein the antibody comprises a heavy chain sequence of SEQ ID NO: 9 and a light chain sequence of SEQ ID NO: 10.

16. The composition according to item 14 or item 15, wherein the composition comprises one or a combination of oxidation at M248 of SEQ ID NO: 9, oxidation at M354 of SEQ ID NO: 9 and/or oxidation at M424 of SEQ ID NO: 9.

17. The composition according to any one of items 14-16, wherein the composition comprises:

(i) ≤65%, 60%, ≤50%, ≤45%, ≤40%, ≤35%, ≤30%, ≤20%, ≤15%, ≤10%, ≤5%, ≤4%, or ≤3% oxidation at M248 of SEQ ID NO: 9,

(ii) ≤65%, 60%, ≤50%, ≤45%, ≤40%, ≤35%, ≤30%, ≤20%, ≤15%, ≤10%, ≤5%, 54%, or ≤3% oxidation at M354 of SEQ ID NO: 9; and/or

(iii) ≤65%, 60%, ≤50%, ≤45%, ≤40%, ≤35%, ≤30%, ≤20%, ≤15%, ≤10%, ≤5%, 54%, or ≤3% oxidation at M424 of SEQ ID NO: 9.

18. The composition according to any one of items 14-17, wherein the amount of oxidation at M248 in the composition is between the lower limit of detection of the method used to identify oxidation at M248 and 65%, 60%, 50%, 40%, 30%, 20%, 15%, 10%, 5%, 4% or 3%.

19. The composition according to any one of items 14-18, wherein the composition comprises oxidation at M248 in an amount:
(i) selected from any one of the following ranges: 0.01-65%, 0.01-60%, 0.01-50%, 0.01-40%, 0.01-30%, 0.01-20%, 0.01-15%, 0.01-10%, 0.01-5%, 0.01-4%, 0.01-3%, 0.05-65%, 0.05-60%, 0.05-50%, 0.05-40%, 0.05-30%, 0.05-20%, 0.05-15%, 0.05-10%, 0.05-5%, 0.05-4%, 0.05-3%, 0.1-65%, 0.1-60%, 0.1-50%, 0.1-40%, 0.1-30%, 0.1-20%, 0.1-15%, 0.1-10%, 0.1-5%, 0.1-4%, 0.1-3%, 0.5-65%, 0.5-60%, 0.5-50%, 0.5-40%, 0.5-30%, 0.5-20%, 0.5-15%, 0.5-10%, 0.5-5%, 0.5-4%, 0.5-3%, 1-65%, 1-60%, 1-50%, 1-40%, 1-30%, 1-20%, 1-15%, 1-10%, 1-5%, 1-4%, 1-3%, 2-4%, and 2-3%; or
(ii) about 10%, about 5%, about 4%, about 3%, about 2%, or about 1%.

20. The composition according to any one of items 14-19, wherein the amount of oxidation at M354 in the composition is between the lower limit of detection of the method used to identify oxidation at M354 and 65%, 60%, 50%, 40%, 30%, 20%, 15%, 10%, 5%, 4% or 3%.

21. The composition according to any one of items 14-20, wherein the composition comprises oxidation at M354 in an amount:
(i) selected from any one of the following ranges: 0.01-65%, 0.01-60%, 0.01-50%, 0.01-40%, 0.01-30%, 0.01-20%, 0.01-15%, 0.01-10%, 0.01-5%, 0.01-4%, 0.01-3%, 0.05-65%, 0.05-60%, 0.05-50%, 0.05-40%, 0.05-30%, 0.05-20%, 0.05-15%, 0.05-10%, 0.05-5%, 0.05-4%, 0.05-3%, 0.1-65%, 0.1-60%, 0.1-50%, 0.1-40%, 0.1-30%, 0.1-20%, 0.1-15%, 0.1-10%, 0.1-5%, 0.1-4%, 0.1-3%, 0.5-65%, 0.5-60%, 0.5-50%, 0.5-40%, 0.5-30%, 0.5-20%, 0.5-15%, 0.5-10%, 0.5-5%, 0.5-4%, 0.5-3%, 1-65%, 1-60%, 1-50%, 1-40%, 1-30%, 1-20%, 1-15%, 1-10%, 1-5%, 1-4%, 1-3%, 2-4%, and 2-3%; or
(ii) about 10%, about 5%, about 4%, about 3%, about 2%, or about 1%.

22. The composition according to any one of items 14-21, wherein the amount of oxidation at M242 in the composition is between the lower limit of detection of the method used to identify oxidation at M424 and 65%, 60%, 50%, 40%, 30%, 20%, 15%, 10%, 5%, 4% or 3%.

23. The composition according to any one of items 14-22, wherein the composition comprises oxidation at M424 in an amount:
(i) selected from any one of the following ranges: 0.01-65%, 0.01-60%, 0.01-50%, 0.01-40%, 0.01-30%, 0.01-20%, 0.01-15%, 0.01-10%, 0.01-5%, 0.01-4%, 0.01-3%, 0.05-65%, 0.05-60%, 0.05-50%, 0.05-40%, 0.05-30%, 0.05-20%, 0.05-15%, 0.05-10%, 0.05-5%, 0.05-4%, 0.05-3%, 0.1-65%, 0.1-60%, 0.1-50%, 0.1-40%, 0.1-30%, 0.1-20%, 0.1-15%, 0.1-10%, 0.1-5%, 0.1-4%, 0.1-3%, 0.5-65%, 0.5-60%, 0.5-50%, 0.5-40%, 0.5-30%, 0.5-20%, 0.5-15%, 0.5-10%, 0.5-5%, 0.5-4%, 0.5-3%, 1-65%, 1-60%, 1-50%, 1-40%, 1-30%, 1-20%, 1-15%, 1-10%, 1-5%, 1-4%, 1-3%, 2-4%, and 2-3%; or
(ii) about 10%, about 5%, about 4%, about 3%, about 2%, or about 1%.

24. The composition according to any one of items 2, 7, 9, 11, 18, 20 or 22, wherein the method used to identify the oxidized variant or oxidation at a specific position is peptide mapping tandem mass spectrometry, optionally as set out in Example 1.

25. The composition according to item 24, wherein the method comprises:

(i) denaturing, reducing, alkylating and digesting the anti-PD-1 antibody; and

(ii) carrying out liquid chromatography with tandem mass spectrometry.

26. The composition according to item 24 or item 25, wherein the method comprises:

(i) denaturing the anti-PD-1 antibody with guanidine hydrochloride, reducing with dithiothreitol, alkylating with iodoacetamide, digesting with endoproteinase Lys-C or trypsin at 37° C. for 4 hours, quenching with trifluoroacetic acid; and

(ii) carrying out ultra-high performance liquid chromatography with tandem electrospray ionization mass spectrometry.

27. A composition comprising an aggregated variant of an anti-PD-1 antibody, wherein the aggregated variant comprises a heavy chain sequence comprising a CDRH1 of SEQ ID NO: 1, a CDRH2 of SEQ ID NO: 2, and a CDRH3 of SEQ ID NO: 3, and a light chain sequence comprising a CDRL1 of SEQ ID NO: 4, a CDRL2 of SEQ ID NO: 5, and a CDRL3 of SEQ ID NO: 6; wherein the composition comprises ≤36%, ≤35%, ≤30%, ≤26%, ≤25%, ≤20%, ≤10%, ≤5%, ≤4%, ≤3%, ≤2%, or ≤1% aggregated variant.

28. The composition according to item 27, wherein the amount of aggregated variant in the composition is between the lower limit of detection of the method used to identify the aggregated variant and 36%, 35%, 30%, 26%, 25%, 20%, 10%, 5%, 4%, 3%, 2%, or 1%.

29. The composition according to item 27 or item 28, comprising the aggregated variant in an amount:
(i) selected from any one of the following ranges: 0.01-36%, 0.01-35%, 0.01-30%, 0.01-26%, 0.01-25%, 0.01-20%, 0.01-10%, 0.01-5%, 0.01-4%, 0.01-3%, 0.01-2%, 0.01-1%, 0.05-36%, 0.05-35%, 0.05-30%, 0.05-26%, 0.05-25%, 0.05-20%, 0.05-10%, 0.05-5%, 0.05-4%, 0.05-3%, 0.05-2%, 0.05-1%; 0.1-36%, 0.1-35%, 0.1-30%, 0.1-26%, 0.1-25%, 0.1-20%, 0.1-10%, 0.1-5%, 0.1-4%, 0.1-3%, 0.1-2%, 0.1-1%, 0.5-36%, 0.5-35%, 0.5-30%, 0.5-26%, 0.5-25%, 0.5-20%, 0.5-10%, 0.5-5%, 0.5-4%, 0.5-3%, 0.5-2%, and 0.5-1%; or
(ii) about 10%, about 5%, about 4%, about 3%, about 2%, or about 1%.

30. The composition according to any one of items 27-29, wherein the antibody comprises a heavy chain sequence of SEQ ID NO: 9 and a light chain sequence of SEQ ID NO: 10.

31. The composition according to any one of items 27-30, wherein the method used to identify the aggregated variant is size exclusion chromatography.

32. The composition according to any one of items 27-31, wherein the method used to identify the aggregated variant is SE-HPLC, optionally as set out in Example 5.1.

33. A composition comprising an antibody having a heavy chain sequence of SEQ ID NO: 9 and a light chain sequence of SEQ ID NO: 10, wherein the composition comprises (i) ≤65%, ≤60%, ≤50%, ≤40%, ≤30%, ≤20%, ≤15%, ≤10%, ≤5%, ≤4% or ≤3% oxidized variant; and/or (ii) ≤36%, ≤35%, ≤30%, ≤26%, ≤25%, ≤20%, ≤10%, ≤5%, ≤4%, ≤3%, ≤2%, or ≤1% aggregated variant.

34. The composition according to item 33, wherein the amount of oxidized variant in the composition is between the lower limit of detection of the method used to identify the oxidized variant and 65%, 60%, 50%, 40%, 30%, 20%, 15%, 10%, 5%, 4% or 3%.

35. The composition according to item 33 or item 34, comprising the oxidized variant in an amount:
(i) selected from any one of the following ranges: 0.01-65%, 0.01-60%, 0.01-50%, 0.01-40%, 0.01-30%, 0.01-20%, 0.01-15%, 0.01-10%, 0.01-5%, 0.01-4%, 0.01-3%, 0.05-65%, 0.05-60%, 0.05-50%, 0.05-40%, 0.05-30%, 0.05-20%, 0.05-15%, 0.05-10%, 0.05-5%, 0.05-4%, 0.05-3%, 0.1-65%, 0.1-60%, 0.1-50%, 0.1-40%, 0.1-30%, 0.1-20%, 0.1-15%, 0.1-10%, 0.1-5%, 0.1-4%, 0.1-3%, 0.5-65%, 0.5-60%, 0.5-50%, 0.5-40%, 0.5-30%, 0.5-20%, 0.5-15%, 0.5-10%, 0.5-5%, 0.5-4%, 0.5-3%, 1-65%, 1-60%, 1-50%, 1-40%, 1-30%, 1-20%, 1-15%, 1-10%, 1-5%, 1-4%, 1-3%, 2-4%, and 2-3%; or
(ii) about 10%, about 5%, about 4%, about 3%, about 2%, or about 1%.

36. The composition according to item 34 or item 35, wherein the method used to identify the oxidized variant is peptide mapping tandem mass spectrometry, optionally as set out in Example 1.

37. The composition according to any one of items 34-36, wherein the method comprises:

(i) denaturing, reducing, alkylating and digesting the anti-PD-1 antibody; and

(ii) carrying out liquid chromatography with tandem mass spectrometry.

38. The composition according to any one of items 34-37, wherein the method comprises:

(i) denaturing the anti-PD-1 antibody with guanidine hydrochloride, reducing with dithiothreitol, alkylating with iodoacetamide, digesting with endoproteinase Lys-C or trypsin at 37° C. for 4 hours, quenching with trifluoroacetic acid; and

(ii) carrying out ultra-high performance liquid chromatography with tandem electrospray ionization mass spectrometry.

39. The composition according to any one of items 33-38, wherein the amount of aggregated variant in the composition is between the lower limit of detection of the method used to identify the aggregated variant and 36%, 35%, 30%, 26%, 25%, 20%, 10%, 5%, 4%, 3%, 2%, or 1%.

40. The composition according to any one of items 33-39, comprising the aggregated variant in an amount:
(i) selected from any one of the following ranges: 0.01-36%, 0.01-35%, 0.01-30%, 0.01-26%, 0.01-25%, 0.01-20%, 0.01-10%, 0.01-5%, 0.01-4%, 0.01-3%, 0.01-2%, 0.01-1%, 0.05-36%, 0.05-35%, 0.05-30%, 0.05-26%, 0.05-25%, 0.05-20%, 0.05-10%, 0.05-5%, 0.05-4%, 0.05-3%, 0.05-2%, 0.05-1%; 0.1-36%, 0.1-35%, 0.1-30%, 0.1-26%, 0.1-25%, 0.1-20%, 0.1-10%, 0.1-5%, 0.1-4%, 0.1-3%, 0.1-2%, 0.1-1%, 0.5-36%, 0.5-35%, 0.5-30%, 0.5-26%, 0.5-25%, 0.5-20%, 0.5-10%, 0.5-5%, 0.5-4%, 0.5-3%, 0.5-2%, and 0.5-1%; or
(ii) about 10%, about 5%, about 4%, about 3%, about 2%, or about 1%.

41. The composition according to item 39 or item 40, wherein the method used to identify the aggregated variant is size exclusion chromatography.

42. The composition according to any one of items 39-41, wherein the method used to identify the aggregated variant is SE-HPLC, optionally as set out in Example 5.1.

43. A composition comprising a charged variant of an anti-PD-1 antibody comprising a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises:

(i) ≤100%, ≤90%, ≤80%, ≤70%, ≤60%, ≤50%, ≤40%, ≤35%, ≤30% or ≤25% acidic variant; and/or

(ii) ≤35%, ≤30%, ≤25%, ≤20%, ≤15%, ≤10%, ≤8%, ≤7.5%, ≤7%, ≤6% or 55% basic variant; and/or

(iii) ≥1%, ≥2.6%, ≥3%, 5%, ≥10%, ≥20%, ≥30%, ≥40%, ≥50%, ≥55%, ≥60%, ≥65%, ≥70%, ≥75%, ≥80% or ≥90% main isoform.

44. The composition according to item 43, wherein the amount of acidic variant in the composition is between the lower limit of detection of the method used to identify the acidic variant and 100%, 90%, 80%, 70%, 60%, 50%, 40%, 35%, 30% or 25%.

45. The composition according to item 43 or 44, wherein the composition comprises an acidic variant in an amount:
(i) selected from any one of the following ranges: 5-100%, 5-90%, 5-80%, 5-70%, 5-60%, 5-50%, 5-40%, 5-35%, 5-30%, 5-25%, 10-100%, 10-97%, 10-90%, 10-80%, 10-70%, 10-60%, 10-50%, 10-40%, 10-35%, 10-30%, 10-25%, 20-100%, 20-97%, 20-90%, 20-80%, 20-70%, 20-60%, 20-50%, 20-40%, 20-35%, 20-30% and 20-25%; or
(ii) about 60%, about 50%, about 45%, about 40%, about 35%, about 30%, about 25%, about 20% or about 10%.

46. The composition according to any one of items 43-45, wherein the amount of basic variant in the composition is between the lower limit of detection of the method used to identify the basic variant and 35%, 30%, 25%, 20%, 15%, 10%, 8%, 7.5%, 7%, 6% or 5%.

47. The composition according to any one of items 43-46, wherein the composition comprises basic variant in an amount:
(i) selected from any one of the following ranges: 0.1-35%, 0.1-30%, 0.1-25%, 0.1-20%, 0.1-15%, 0.1-10%, 0.1-8%, 0.1-7.5%, 0.1-7%, 0.1-6%, 0.1-5%, 1-35%, 1-30%, 1-25%, 1-20%, 1-15%, 1-10%, 1-8%, 1-7.5%, 1-7%, 1-6% and 1-5%; or
(ii) about 35%, about 30%, about 25%, about 20%, about 15%, about 10%, about 7.5% or about 5%.

48. The composition according to any one of items 43-47, wherein the composition comprises main form in an amount:
(i) selected from any one of the following ranges: 2-90%, 2-80%, 2-75%, 5-90%, 10-90%, 20-90%, 30-90%, 40-90%, 50-90%, 60-90%, 5-80%, 10-80%, 20-80%, 30-80%, 40-80%, 50-80% and 60-80%; or
(ii) about 80%, about 75%, about 70%, about 65%, about 60%, about 50% or about 55%.

49. A composition comprising a charged variant of an anti-PD-1 antibody comprising a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises: 10-97% acidic variant; and/or 0.1-35% basic variant; and/or 2-80% main isoform.

50. A composition comprising a charged variant of an anti-PD-1 antibody comprising a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises: ≤35% acidic variant; and/or ≤5% basic variant; and/or ≥55% main isoform.

51. A composition comprising a charged variant of an anti-PD-1 antibody comprising a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises: 10-30% acidic variant; and/or 0.1-10% basic variant; and/or 60-80% main isoform.

52. The composition according to any one of items 43-51, wherein the percent acidic variant, percent basic variant and percent main isoform of the composition is determined using capillary isoelectric focusing.

53. The composition according to any one of the preceding items, wherein the composition comprises a deamidated variant.

54. The composition according to item 53, wherein the deamidated variant comprises a deamidated residue selected from: an aspartic acid residue, a succinimide-aspartic acid residue, and an iso-aspartic acid residue.

55. The composition according to item 54, wherein the deamidated variant comprises up to 100% deamidation at N380 and/or N385 of SEQ ID NO: 9.

56. The composition according to item 54, wherein the deamidated variant comprises 0.1-100%, 0.1-90%, 0.1-80%, 0.1-70%, 0.1-60%, 0.1-50%, 0.1-40%, 0.1-30%, 0.1-20%, 0.1-10%, 1-100%, 1-90%, 1-80%, 1-70%, 1-60%, 1-50%, 1-40%, 1-30%, 1-20%, 1-10%, 2-100%, 3-100%, 4-100%, 5-100%, 6-100%, 7-100%, 8-100%, 9-100%, 2-30%, 3-30%, 4-30%, 5-30%, 2-40%, 3-40%, 4-40%, 5-40%, 2-10%, 3-10%, 4-10%, or 5-9% deamidation at N380.

57. The composition according to item 54, wherein the deamidated variant comprises 1% or more, 2% or more, 3% or more, 4% or more, 5% or more, 6% or more, 7% or more, 8% or more, 9% or more, or 10% or more deamidation at N380.

58. The composition according to any one of items 54-57, wherein the deamidated variant comprises 0.1-100%, 0.1-90%, 0.1-80%, 0.1-70%, 0.1-60%, 0.1-50%, 0.1-40%, 0.1-30%, 0.1-20%, 0.1-10%, 1-100%, 1-90%, 1-80%, 1-70%, 1-60%, 1-50%, 1-40%, 1-30%, 1-20%, 1-10%, 2-100%, 3-100%, 4-100%, 5-100%, 6-100%, 7-100%, 8-100%, 9-100%, 2-30%, 3-30%, 4-30%, 5-30%, 2-40%, 3-40%, 4-40%, 5-40%, 2-10%, 3-10%, 4-10%, or 5-9% deamidation at N385.

59. The composition according to any one of items 54-58, wherein the deamidated variant comprises 0.5% or more, 1% or more, 2% or more, 3% or more, 4% or more, 5% or more, 6% or more, 7% or more, 8% or more, 9% or more, or 10% or more deamidation at N385.

60. The composition according to any one of items 53-59, wherein the deamidated variant comprises a sequence of SEQ ID NO: 11, SEQ ID NO: 12 or SEQ ID NO: 13.

61. The composition according to any one of the preceding items, wherein the composition comprises an isomerized variant.

62. The composition according to item 61, wherein the composition comprises up to 100% isomerization at D147 of SEQ ID NO: 9.

63. The composition according to item 61 or item 62, wherein the composition comprises 0.1-90%, 0.1-80%, 0.1-70%, 0.1-60%, 0.1-50%, 0.1-40%, 0.1-30%, 0.1-20%, 0.1-15%, 0.1-10%, 1-100%, 1-90%, 1-80%, 1-70%, 1-60%, 1-50%, 1-40%, 1-30%, 1-20%, 1-15% or 1-10% isomerized variant.

64. The composition according to any one of the preceding items, wherein the composition comprises up to 100% heavy chain N-terminal pyro-glutamate variant and/or up to 100% heavy chain C-terminal lysine cleaved variant.

65. The composition according to any one of the preceding items, wherein the composition comprises 0.1-100%, 0.1-90%, 0.1-80%, 0.1-70%, 0.1-60%, 0.1-50%, 0.1-40%, 0.1-30%, 0.1-20%, 0.1-10%, 0.1-9%, 0.1-8%, 0.1-7%, 0.1-6%, 0.1-5%, 0.1-4%, 0.1-3%, 0.1-2%, 0.1-1%, 1-100%, 1-90%, 1-80%, 1-70%, 1-60%, 1-50%, 1-40%, 1-30%, 1-20%, 1-10%, 1-9%, 1-8%, 1-7%, 1-6%, 1-5%, 1-4%, 1-3%, or 1-2% heavy chain N-terminal pyro-glutamate variant.

66. The composition according to any one of the preceding items, wherein the composition comprises ≥10%, ≥20%, ≥30%, ≥40%, ≥50%, ≥60%, ≥70%, ≥80%, ≥90% or ≥95% heavy chain C-terminal lysine cleaved variant.

67. The composition according to any one of the preceding items, wherein the composition comprises 1-100%, 10-100%, 20-100%, 30-100%, 40-100%, 50-100%, 60-100%, 70-100%, 80-100%, 90-100%, 95-99%, 96-99% or 97-99% heavy chain C-terminal lysine cleaved variant.

68. A composition comprising an antibody comprising a heavy chain sequence having one or a combination of sequences selected from SEQ ID NO: 9, SEQ ID NO: 11, SEQ ID NO: 12 and/or SEQ ID NO: 13, and a light chain sequence of SEQ ID NO: 10, wherein the composition comprises ≤64%, ≤60%, ≤50%, ≤40%, ≤30%, ≤20%, ≤15%, ≤10%, ≤5%, ≤4%, or ≤3% of oxidized variant.

69. The composition according to item 68, wherein the amount of oxidized variant in the composition is between the lower limit of detection of the method used to identify the oxidized variant and 65%, 60%, 50%, 40%, 30%, 20%, 15%, 10%, 5%, 4% or 3%.

70. The composition according to item 68 or item 69, comprising the oxidized variant in an amount:
(i) selected from any one of the following ranges: 0.01-65%, 0.01-60%, 0.01-50%, 0.01-40%, 0.01-30%, 0.01-20%, 0.01-15%, 0.01-10%, 0.01-5%, 0.01-4%, 0.01-3%, 0.05-65%, 0.05-60%, 0.05-50%, 0.05-40%, 0.05-30%, 0.05-20%, 0.05-15%, 0.05-10%, 0.05-5%, 0.05-4%, 0.05-3%, 0.1-65%, 0.1-60%, 0.1-50%, 0.1-40%, 0.1-30%, 0.1-20%, 0.1-15%, 0.1-10%, 0.1-5%, 0.1-4%, 0.1-3%, 0.5-65%, 0.5-60%, 0.5-50%, 0.5-40%, 0.5-30%, 0.5-20%, 0.5-15%, 0.5-10%, 0.5-5%, 0.5-4%, 0.5-3%, 1-65%, 1-60%, 1-50%, 1-40%, 1-30%, 1-20%, 1-15%, 1-10%, 1-5%, 1-4%, 1-3%, 2-4%, and 2-3%; or
(ii) about 10%, about 5%, about 4%, about 3%, about 2%, or about 1%.

71. The composition according to any one of items 68-70, wherein the method used to identify the oxidized variant is peptide mapping tandem mass spectrometry, optionally as set out in Example 1.

72. The composition according to any one of items 68-71, wherein the method comprises:

(i) denaturing, reducing, alkylating and digesting the anti-PD-1 antibody; and

(ii) carrying out liquid chromatography with tandem mass spectrometry.

73. The composition according to any one of items 68-72, wherein the method comprises:

(i) denaturing the anti-PD-1 antibody with guanidine hydrochloride, reducing with dithiothreitol, alkylating with iodoacetamide, digesting with endoproteinase Lys-C or trypsin at 37° C. for 4 hours, quenching with trifluoroacetic acid; and

(ii) carrying out ultra-high performance liquid chromatography with tandem electrospray ionization mass spectrometry.

74. A composition comprising an antibody comprising a heavy chain sequence having one or a combination of sequences selected from SEQ ID NO: 9, SEQ ID NO: 11, SEQ ID NO: 12 and/or SEQ ID NO: 13, and a light chain sequence of SEQ ID NO: 10, wherein the composition comprises ≤36%, ≤35%, ≤30%, ≤26%, ≤25%, ≤20%, ≤10%, ≤5%, ≤4%, ≤3%, ≤2%, or ≤1% aggregated variant.

75. The composition according to item 74, wherein the amount of aggregated variant in the composition is between the lower limit of detection of the method used to identify the aggregated variant and 36%, 35%, 30%, 26%, 25%, 20%, 10%, 5%, 4%, 3%, 2%, or 1%.

76. The composition according to item 74 or item 75, comprising the aggregated variant in an amount:
(i) selected from any one of the following ranges: 0.01-36%, 0.01-35%, 0.01-30%, 0.01-26%, 0.01-25%, 0.01-20%, 0.01-10%, 0.01-5%, 0.01-4%, 0.01-3%, 0.01-2%, 0.01-1%, 0.05-36%, 0.05-35%, 0.05-30%, 0.05-26%, 0.05-25%, 0.05-20%, 0.05-10%, 0.05-5%, 0.05-4%, 0.05-3%, 0.05-2%, 0.05-1%; 0.1-36%, 0.1-35%, 0.1-30%, 0.1-26%, 0.1-25%, 0.1-20%, 0.1-10%, 0.1-5%, 0.1-4%, 0.1-3%, 0.1-2%, 0.1-1%, 0.5-36%, 0.5-35%, 0.5-30%, 0.5-26%, 0.5-25%, 0.5-20%, 0.5-10%, 0.5-5%, 0.5-4%, 0.5-3%, 0.5-2%, and 0.5-1%; or
(ii) about 10%, about 5%, about 4%, about 3%, about 2%, or about 1%.

77. The composition according to any one of items 74-76, wherein the method used to identify the aggregated variant is size exclusion chromatography.

78. The composition according to any one of items 27-31, wherein the method used to identify the aggregated variant is SE-HPLC, optionally as set out in Example 5.1.

79. A composition comprising a variant of an anti-PD-1 antibody, wherein the variant comprises a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition has at least 60% of the potency of a composition comprising a heavy chain sequence of SEQ ID NO: 9 and a light chain sequence of SEQ ID NO: 10, 10-97% acidic variant, 0.1-35% basic variant, 2-80% main isoform, 4.8% or less light chain W50 oxidized variant, 1% or less heavy chain M34 oxidized variant, 1.2% or less heavy chain M103 oxidized variant, 15.2% or less aggregated variant, 16.7% or less heavy chain M354 oxidized variant, 29.0% or less heavy chain M424 oxidized variant, 47.1% or less heavy chain M248 oxidized variant, 20.8% or less heavy chain D147 isomerized variant, 13.1% or less heavy chain D151 or D167 isomerized variant, 3.1% or less heavy chain D261, D266 or D276 isomerization variant, 4.6% or less fragmented variants, 27.8% or less heavy chain N380 deamidated variant, 27.2% or less heavy chain N385 deamidated variant, about 7.4% or less heavy chain N311 deamidated variant, about 2.0% or less heavy chain N430 deamidated variant, 90% or more heavy chain C-terminal lysine deleted variants (ΔK443), and 1% or less heavy chain N-terminal pyro-glutamate variant.

80. A composition comprising a variant of an anti-PD-1 antibody, wherein the variant comprises a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition has at least 60% of the potency of a composition comprising a heavy chain sequence of SEQ ID NO: 9 and a light chain sequence of SEQ ID NO: 10, 10-97% acidic variant, 0.1-35% basic variant, 2-80% main isoform, 0.01-4.8% light chain W50 oxidized variant, 0.01-1% heavy chain M34 oxidized variant, 0.01-1.2% heavy chain M103 oxidized variant, 0.01-15.2% aggregated variant, 0.01-16.7% heavy chain M354 oxidized variant, 0.01-29.0% heavy chain M424 oxidized variant, 0.01-47.1% heavy chain M248 oxidized variant, 0.01-20.8% heavy chain D147 isomerized variant, 0.01-13.1% heavy chain D151 or D167 isomerized variant, 0.01-3.1% heavy chain D261, D266 or D276 isomerization variant, 0.01-4.6% fragmented variants, 0.01-27.8% heavy chain N380 deamidated variant, 0.01-27.2% heavy chain N385 deamidated variant, about 0.01-7.4% heavy chain N311 deamidated variant, about 0.01-2.0% heavy chain N430 deamidated variant, 90% or more heavy chain C-terminal lysine deleted variants (ΔK443), and 0.01-1% heavy chain N-terminal pyro-glutamate variant.

81. The composition according to any one of the preceding items, wherein the antibody is a full-length antibody.

82. The composition according to any one of the preceding items, wherein the antibody is humanized.

83. The composition according to any one of the preceding items, which is formed during the manufacture or storage of the antibody.

84. A pharmaceutical composition comprising the composition according to any one of the preceding items and at least one pharmaceutically acceptable excipient.

85. A formulation comprising the pharmaceutical composition according to item 82, comprising the antibody at about 20 mg/mL to about 125 mg/mL and a buffering agent at a pH of about 5.5 to about 6.5.

86. The formulation according to item 85, wherein the buffering agent is selected from citrate buffer or histidine buffer.

87. The formulation according to item 85 or item 86, wherein the buffering agent is citrate buffer at a pH of about 6.0.

88. The formulation according to any one of items 85-87, which additionally comprises arginine and/or trehalose.

89. The formulation according to any one of items 85-88, which additionally comprises polysorbate 80.

90. The formulation according to any one of items 85-89, which additionally comprises sodium chloride at a concentration to adjust the osmolality of the formulation to about 290-325 mOsm/kg.

91. A formulation comprising the pharmaceutical composition according to item 84, comprising: (a) the antibody at about 20 mg/mL to about 125 mg/mL, (b) citrate buffer or histidine buffer at about 10 mM to about 40 mM, (c) arginine at about 80 mM to about 120 mM or trehalose at about 2 to about 10% w/v, (d) sodium chloride at about 20 mM to about 40 mM, and (e) polysorbate 80 at about 0.01% to about 0.1% w/v, at a pH of about 5.5 to about 6.5.

92. The formulation according to item 91, comprising about 20 mg/mL of the antibody, about 25 mM citrate buffer, about 100 mM arginine, about 31 mM sodium chloride, and about 0.02% w/v polysorbate 80, at about pH 6.

93. The formulation according to item 91, comprising about 50 mg/mL of the antibody, about 25 mM citrate buffer, about 100 mM arginine, about 31 mM sodium chloride, and about 0.02% (w/v) polysorbate 80, at about pH 6.

94. An injection device comprising the composition according to any one of items 1-83, the pharmaceutical composition according to item 84 or the formulation according to any one of items 85-94.

95. A cell culture medium comprising the composition according to any one of items 1-83.

96. An eluate comprising the composition according to any one of items 1-83.

97. A method of treating cancer comprising administering to a subject in need thereof a therapeutically effective amount of the composition according to any one of items 1-83, the pharmaceutical composition according to item 84, or the formulation according to any one of items 85-93.

98. The method according to item 97, wherein the composition is administered at a dose of about 500 mg.

99. The method according to item 98, wherein the composition is administered once every 3 weeks.

100. The method according to item 98 or item 99, wherein the composition is administered for 4 cycles.

101. The method according to item 97, wherein the composition is administered at a first dose of about 500 mg once every 3 weeks for 4 cycles followed by a second dose of about 1000 mg once every 6 weeks or more.

102. The method according to item 101, wherein the second dose of about 1000 mg once every 6 weeks or more is continued to maintain clinical benefit.

103. A composition according to any one of items 1-83, the pharmaceutical composition according to item 84, or the formulation according to any one of items 84-93 for use in therapy.

104. A composition according to any one of items 1-83, the pharmaceutical composition according to item 84, or the formulation according to any one of items 85-93 for use in the treatment of cancer.

105. Use of a composition according to any one of items 1-83, the pharmaceutical composition according to item 84, or the formulation according to any one of items 85-93 in the manufacture of a medicament for use in the treatment of cancer.

SEQUENCES

SEQ ID NO: 1-CDRH1
SYDMS
SEQ ID NO: 2-CDRH2
TISGGGSYTYYQDSVKG
SEQ ID NO: 3-CDRH3
PYYAMDY
SEQ ID NO: 4-CDRL1
KASQDVGTAVA
SEQ ID NO: 5-CDRL2
WASTLHT
SEQ ID NO: 6-CDRL3
QHYSSYPWT
SEQ ID NO: 7-Heavy chain variable region
(CDRs are underlined)
EVQLLESGGGLVQPGGSLRLSCAASGFTFSSYDMSWVRQAPGKGLEWVS
TISGGGSYTYYQDSVKGRFTISRDNSKNTLYLQMNSLRAEDTAVYYCAS
PYYAMDYWGQGTTVTVSS
SEQ ID NO: 8-Light chain variable region
(CDRs are underlined)
DIQLTQSPSFLSAYVGDRVTITCKASQDVGTAVAWYQQKPGKAPKLLIY
WASTLHTGVPSRFSGSGSGTEFTLTISSLQPEDFATYYCQHYSSYPWTF
GQGTKLEIK
SEQ ID NO: 9-Full heavy chain sequence
(CDRs are underlined)
EVQLLESGGGLVQPGGSLRLSCAASGFTFSSYDMSWVRQAPGKGLEWVS
TISGGGSYTYYQDSVKGRFTISRDNSKNTLYLQMNSLRAEDTAVYYCAS
PYYAMDYWGQGTTVTVSSASTKGPSVFPLAPCSRSTSESTAALGCLVKD
YFPEPVTVSWNSGALTSGVHTFPAVLQSSGLYSLSSVVTVPSSSLGTKT
YTCNVDHKPSNTKVDKRVESKYGPPCPPCPAPEFLGGPSVFLFPPKPKD
TLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQ
VYTLPPSQEEMTKNQVSLTCLVKGFYPSDIAVEWESNGQPENNYKTTPP
VLDSDGSFFLYSRLTVDKSRWQEGNVFSCSVMHEALHNHYTQKSLSLSL
GK
SEQ ID NO: 10-Full light chain sequence
(CDRs are underlined)
DIQLTQSPSFLSAYVGDRVTITCKASQDVGTAVAWYQQKPGKAPKLLIY
WASTLHTGVPSRFSGSGSGTEFTLTISSLQPEDFATYYCQHYSSYPWTF
GQGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC
SEQ ID NO: 11-Full heavy chain sequence with
N380D modification (CDRs are underlined)
EVQLLESGGGLVQPGGSLRLSCAASGFTFSSYDMSWVRQAPGKGLEWVS
TISGGGSYTYYQDSVKGRFTISRDNSKNTLYLQMNSLRAEDTAVYYCAS
PYYAMDYWGQGTTVTVSSASTKGPSVFPLAPCSRSTSESTAALGCLVKD
YFPEPVTVSWNSGALTSGVHTFPAVLQSSGLYSLSSVVTVPSSSLGTKT
YTCNVDHKPSNTKVDKRVESKYGPPCPPCPAPEFLGGPSVFLFPPKPKD
TLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQ
VYTLPPSQEEMTKNQVSLTCLVKGFYPSDIAVEWESDGQPENNYKTTPP
VLDSDGSFFLYSRLTVDKSRWQEGNVFSCSVMHEALHNHYTQKSLSLSL
GK
SEQ ID NO: 12-Full heavy chain sequence with
N385D modification (CDRs are underlined)
EVQLLESGGGLVQPGGSLRLSCAASGFTFSSYDMSWVRQAPGKGLEWVS
TISGGGSYTYYQDSVKGRFTISRDNSKNTLYLQMNSLRAEDTAVYYCAS
PYYAMDYWGQGTTVTVSSASTKGPSVFPLAPCSRSTSESTAALGCLVKD
YFPEPVTVSWNSGALTSGVHTFPAVLQSSGLYSLSSVVTVPSSSLGTKT
YTCNVDHKPSNTKVDKRVESKYGPPCPPCPAPEFLGGPSVFLFPPKPKD
TLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQ
VYTLPPSQEEMTKNQVSLTCLVKGFYPSDIAVEWESNGQPEDNYKTTPP
VLDSDGSFFLYSRLTVDKSRWQEGNVFSCSVMHEALHNHYTQKSLSLSL
GK
SEQ ID NO: 13-Full heavy chain sequence with
N380D and N385D modifications
(CDRs are underlined)
EVQLLESGGGLVQPGGSLRLSCAASGFTFSSYDMSWVRQAPGKGLEWVS
TISGGGSYTYYQDSVKGRFTISRDNSKNTLYLQMNSLRAEDTAVYYCAS
PYYAMDYWGQGTTVTVSSASTKGPSVFPLAPCSRSTSESTAALGCLVKD
YFPEPVTVSWNSGALTSGVHTFPAVLQSSGLYSLSSVVTVPSSSLGTKT
YTCNVDHKPSNTKVDKRVESKYGPPCPPCPAPEFLGGPSVFLFPPKPKD
TLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQ
VYTLPPSQEEMTKNQVSLTCLVKGFYPSDIAVEWESDGQPEDNYKTTPP
VLDSDGSFFLYSRLTVDKSRWQEGNVFSCSVMHEALHNHYTQKSLSLSL
GK
SEQ ID NO: 14--CDRL3 Alternative
QHYNSYPWT

1. A composition comprising an oxidized variant of an anti-PD-1 antibody, wherein the oxidized variant comprises a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises ≤65% of oxidized variant.

2. The composition according to claim 1, wherein the oxidized variant comprises oxidation at a methionine residue, at a tryptophan residue, or at both a methionine reside and a tryptophan residue in any one of SEQ ID NOs: 1-6.

3. The composition according to claim 1, wherein the oxidized variant comprises one or a combination of oxidation at: M34 of CDRH1, M103 of CDRH3 and/of W50 of CDRL2.

4. The composition according to claim 3, wherein the composition comprises one or a combination of: ≤21% oxidation at M34 of CDRH1, ≤64% oxidation at M103 of CDRH3, and ≤34% oxidation at W50 of CDRL2.

5. The composition according to claim 1, wherein the antibody comprises a heavy chain variable region at least about 90% identical to the amino acid sequence of SEQ ID NO: 7 and/Of a light chain variable region at least about 90% identical to the amino acid sequence of SEQ ID NO: 8.

6. The composition according to claim 1, wherein the antibody is at least about 90% identical to the heavy chain amino acid sequence of SEQ ID NO: 9 and/of at least about 90% identical to the light chain amino acid sequence of SEQ ID NO: 10.

7. The composition according to claim 1, wherein the antibody comprises a heavy chain sequence of SEQ ID NO: 9 and a light chain sequence of SEQ ID NO: 10.

8. The composition according to claim 6, wherein the composition comprises one or a combination of: ≤65% oxidation at M248 of SEQ ID NO: 9, ≤65% oxidation at M354 of SEQ ID NO: 9 and ≤65% oxidation at M424 of SEQ ID NO: 9.

9. A composition comprising an aggregated variant of an anti-PD-1 antibody, wherein the aggregated variant comprises a heavy chain sequence comprising a CDRH1 of SEQ ID NO: 1, a CDRH2 of SEQ ID NO: 2, and a CDRH3 of SEQ ID NO: 3, and a light chain sequence comprising a CDRL1 of SEQ ID NO: 4, a CDRL2 of SEQ ID NO: 5, and a CDRL3 of SEQ ID NO: 6; wherein the composition comprises ≤36% aggregated variant.

10. The composition according to claim 9, wherein the antibody comprises a heavy chain sequence of SEQ ID NO: 9 and a light chain sequence of SEQ ID NO: 10.

11. A composition comprising an antibody having a heavy chain sequence of SEQ ID NO: 9 and a light chain sequence of SEQ ID NO: 10, wherein the composition comprises (i) ≤65% oxidized variant; and (ii) ≤36% aggregated variant.

12. A composition comprising a charged variant of an anti-PD-1 antibody comprising a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises: ≤100% acidic variant; and/or ≤35% basic variant; and ≥1% main isoform.

13. A composition comprising a charged variant of an anti-PD-1 antibody comprising a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises: 10-97% acidic variant; and/or 0.1-35% basic variant; and/of 2-80% main isoform.

14. A composition comprising a charged variant of an anti-PD-1 antibody comprising a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition comprises: ≤35% acidic variant; and/or 55% basic variant; and/of ≥55% main isoform.

15. The composition according to claim 12, wherein the percent acidic variant, percent basic variant and percent main isoform of the composition is determined using capillary isoelectric focusing.

16. The composition according to claim 1, wherein the composition comprises a deamidated variant.

17. The composition according to claim 16, wherein the deamidated variant comprises a deamidated residue selected from: an aspartic acid residue, a succinimide-aspartic acid residue, or an iso-aspartic acid residue.

18. The composition according to claim 17, wherein the deamidated variant comprises up to 100% deamidation at N380 and up to 100% deamidation at N385 of SEQ ID NO: 9.

19. The composition according to claim 17, wherein the deamidated variant comprises a sequence of SEQ ID NO: 11, SEQ ID NO: 12 or SEQ ID NO: 13.

20. The composition according to claim 1, wherein the composition comprises an isomerized variant.

21. The composition according to claim 20, wherein the composition comprises up to 100% isomerization at D147 of SEQ ID NO: 9.

22. The composition according to claim 1, wherein the composition comprises up to 100% heavy chain N-terminal pyro-glutamate variant and up to 100% heavy chain C-terminal lysine cleaved variant.

23. A composition comprising an antibody comprising a heavy chain sequence having one or a combination of sequences selected from SEQ ID NO: 9, SEQ ID NO: 11, SEQ ID NO: 12 and/or SEQ ID NO: 13, and a light chain sequence of SEQ ID NO: 10, wherein the composition comprises ≤64% oxidized variant.

24. A composition comprising an antibody comprising a heavy chain sequence having one or a combination of sequences selected from SEQ ID NO: 9, SEQ ID NO: 11, SEQ ID NO: 12 and/or SEQ ID NO: 13, and a light chain sequence of SEQ ID NO: 10, wherein the composition comprises ≤36% aggregated variant.

25. A composition comprising a variant of an anti-PD-1 antibody, wherein the variant comprises a heavy chain amino acid sequence comprising a CDRH1 of SEQ ID NO: 1, a CDRH2 of SEQ ID NO: 2, and a CDRH3 of SEQ ID NO: 3, and a light chain amino acid sequence comprising a CDRL1 of SEQ ID NO: 4, a CDRL2 of SEQ ID NO: 5, and a CDRL3 of SEQ ID NO: 6; wherein the composition has at least 60% of the potency of a composition comprising a heavy chain sequence of SEQ ID NO: 9 and a light chain sequence of SEQ ID NO: 10, 10-97% acidic variant, 0.1-35% basic variant, 2-80% main isoform, 4.8% or less light chain W50 oxidized variant, 1% or less heavy chain M34 oxidized variant, 1.2% or less heavy chain M103 oxidized variant, 15.2% or less aggregated variant, 16.7% or less heavy chain M354 oxidized variant, 29.0% or less heavy chain M424 oxidized variant, 47.1% or less heavy chain M248 oxidized variant, 20.8% or less heavy chain D147 isomerized variant, 13.1% or less heavy chain D151 or D167 isomerized variant, 3.1% or less heavy chain D261, D266 or D276 isomerization variant, 4.6% or less fragmented variants, 27.8% or less heavy chain N380 deamidated variant, 27.2% or less heavy chain N385 deamidated variant, about 7.4% or less heavy chain N311 deamidated variant, about 2.0% or less heavy chain N430 deamidated variant, 90% or more heavy chain C-terminal lysine deleted variants (ΔK443), and 1% or less heavy chain N-terminal pyro-glutamate variant.

26. The composition according to claim 1, wherein the antibody is a full-length antibody.

27. The composition according to claim 1, wherein the antibody is humanized.

28. The composition according to claim 1, which is formed during the manufacture or storage of the antibody.

29. A pharmaceutical composition comprising the composition according to claim 1 and at least one pharmaceutically acceptable excipient.

30. A formulation comprising the pharmaceutical composition according to claim 29, comprising the antibody at about 20 mg/mL to about 125 mg/mL and a buffering agent at a pH of about 5.5 to about 6.5.

31. The formulation according to claim 30, wherein the buffering agent is selected from citrate buffer or histidine buffer.

32. The formulation according to claim 30, wherein the buffering agent is citrate buffer at a pH of about 6.0.

33. The formulation according to claim 30, which additionally comprises arginine and/or trehalose.

34. The formulation according to claim 30, which additionally comprises polysorbate 80.

35. The formulation according to claim 30, which additionally comprises sodium chloride at a concentration to adjust the osmolality of the formulation to about 290-325 mOsm/kg.

36. A formulation comprising the pharmaceutical composition according to claim 29, comprising (a) the antibody at about 20 mg/mL to about 125 mg/mL, (b) citrate buffer or histidine buffer at about 10 mM to about 40 mM, (c) arginine at about 80 mM to about 120 mM or trehalose at about 2 to about 10% w/v, (d) sodium chloride at about 20 mM to about 40 mM, and (e) polysorbate 80 at about 0.01% to about 0.1% w/v, at a pH of about 5.5 to about 6.5.

37. The formulation according to claim 36, comprising about 20 mg/mL of the antibody, about 25 mM citrate buffer, about 100 mM arginine, about 31 mM sodium chloride, and about 0.02% w/v polysorbate 80, at about pH 6.

38. The formulation according to claim 36, comprising about 50 mg/mL of the antibody, about 25 mM citrate buffer, about 100 mM arginine, about 31 mM sodium chloride, and about 0.02% (w/v) polysorbate 80, at about pH 6.

39. An injection device comprising the composition according to claim 26.

40. A cell culture medium comprising the composition according to claim 1.

41. An eluate comprising the composition according to claim 1.

42. A method of treating cancer comprising administering to a subject in need thereof a therapeutically effective amount of the composition according to claim 26.

43. The method according to claim 42, wherein the composition is administered at a dose of about 500 mg.

44. The method according to claim 43, wherein the composition is administered once every 3 weeks.

45. The method according to claim 43, wherein the composition is administered for 4 cycles.

46. The method according to claim 42, wherein the composition is administered at a first dose of about 500 mg once every 3 weeks for 4 cycles followed by a second dose of about 1000 mg once every 6 weeks or more.

47. The method according to claim 46, wherein the second dose of about 1000 mg once every 6 weeks or more is continued to maintain clinical benefit.

48. (canceled)

49. (canceled)

50. (canceled)

51. A method of treating an autoimmune disease comprising administering to a subject in need thereof a therapeutically effective amount of the composition according to claim 26.

52. A method of treating an infectious disease comprising administering to a subject in need thereof a therapeutically effective amount of the composition according to claim 26.
